(19) United States
(12) Patent Application Publication
(10) Pub. No.: US 2020/0116737 A1
(43) Pub. Date: Apr. 16, 2020

(54) BIOMARKER COMBINATIONS FOR MONITORING CHRONIC OBSTRUCTIVE PULMONARY DISEASE AND/OR ASSOCIATED MECHANISMS

(71) Applicant: ProterixBio, Inc., Billerica, MA (US)

(72) Inventors: Brett Masters, Belmont, CA (US); Martin Latterich, San Marcos, CA (US); Julio E. Herrera, Wakefield, MA (US); Michael Miller, Hollis, NH (US)

(21) Appl. No.: 16/597,780

(22) Filed: Oct. 9, 2019

Related U.S. Application Data
(63) Continuation of application No. PCT/US2018/027390, filed on Apr. 12, 2018.
(60) Provisional application No. 62/590,080, filed on Nov. 22, 2017.
(60) Provisional application No. 62/484,565, filed on Apr. 12, 2017.

(51) Int. Cl. G01N 33/68; G01N 33/543

(52) U.S. Cl. CPC G01N 33/6893; G01N 2800/60; G01N 2800/122; G01N 33/54313

(57) ABSTRACT

Provided herein are methods for assessing a disease score of a subject suffering from or suspected to be suffering from chronic obstructive pulmonary disease (COPD) or associated disease mechanisms, wherein the disease score represents COPD activity. The disease score can be used to stratify the subject into a specific risk category and can further inform patient management decisions. The methods can involve determining a biomarker signature including four or more biomarkers associated with COPD or COPD mechanisms. The methods can further include supplementing the biomarker combinations with calculation or classification trees based on one or more additional clinical parameters or biomarkers. In some cases, the methods include timing of collection of patient samples with respect to acute event or treatment course.

### CROSS REFERENCE

This application is a continuation application of International Patent Application No. PCT/US2018/027390, filed Apr. 12, 2018, which claims priority to U.S. Provisional Application No. 62/484,565, filed Apr. 12, 2017 and U.S. Provisional Application No. 62/590,080, filed Nov. 22, 2017, each of which are incorporated herein by reference in their entireties.

### BACKGROUND OF THE INVENTION

Chronic respiratory diseases are collectively one of the major causes of morbidity and mortality in the world. Specifically, chronic obstructive pulmonary disease (COPD) is currently the third leading cause of death in the U.S., affecting more than 5% of the population. Many afflicted with COPD, and many more having early stages of chronic respiratory disease, are undetected or undiagnosed. Additionally, a good number of the population at large is treated for acute or chronic respiratory symptoms without specific cause being identified with confidence. As understood today, COPD is a slowly progressive, highly heterogeneous disease characterized by chronic airway and systemic inflammation, yet interrupted by acute disease exacerbations associated by even higher inflammatory and immune response burden. Increased frequency and severity of COPD exacerbations is strongly associated with high healthcare resource utilization related to frequent clinician visits, loss of productivity and particularly hospitalizations.

Effective early stage detection and monitoring of COPD or COPD associated biological mechanisms, is important to alleviate symptoms, reduce the frequency and severity of exacerbations, improve health status with targeted therapies and care, and prolong survival. Chronic disease arrest, maintenance, and/or prevention of COPD exacerbations or treatment of an exacerbation at the onset are key goals of therapeutic interventions. Most of these interventions are performed on clinical symptomatic grounds, which in many times lead to either delays in therapy or unnecessary interventions (i.e. unnecessary use of antibiotics or steroids).

COPD has been identified as a highly heterogeneous disease and as such many biochemical disease pathways have been investigated across broad populations of patients A further complication is the substantial response of many of the biochemical pathways to the plethora of available treatments to this aging patient population who also experience substantial co-morbidities such as concomitant asthma, hypertension, cardiovascular disease, diabetes, gastrointestinal disorders, osteoporosis, cancer and many others. It is challenging to identify specific disease activity, status, and propensity for eminent clinical events or progression. Unfortunately, few specific combinations of molecular markers, or specific combinations of molecular markers with clinical biomarkers, have been identified to date that can be used, as a metric of disease status, to reliably monitor the time varying nature of the active biochemical pathways of disease, guide therapeutic choices or correlate with disease stability, progression and risks of acute disease exacerbations. Therefore, there is a need to discover and test novel complementary combinations of biomarkers (including clinical and molecular), as measures of disease status, that reliably correlate with past, present and future disease events, indicating associated stability, risks of future events and recent disease control in response to interventions.

### SUMMARY OF THE INVENTION

Described herein, in certain instances, are methods of determining a disease score of a subject having, suspected of having, or at risk of progressing to chronic obstructive pulmonary disease, said method comprising: (a) detecting, from a biological sample from said subject, a level of at least one biomarker selected from a group consisting of: an advanced glycation end-product, a platelet degradation product, a coagulation protein, a protein involved in platelet activity, a degradation product of fibrin, a chemotaxis protein, a chemokine produced by an immune response, an endopeptidase inhibitor, a club cell rated protein, a protein involved with calcium homeostasis, a natriuretic peptide, a pentraxin, a complement pathway protein, an interleukin receptor or receptor-like protein, a toll-like receptor or protein with toll-like receptor domains, an acute phase protein, a cathepsin, a cystatin, a leukocyte or neutrophil related protein, an immunoglobulin, a serpin, a chitinase, an adipokine, an adipose derived hormone, a protein involved in the metabolic pathway, a protein involved with insulin resistance, an immunoglobulin, an eosinophil related protein, a matrix metallopeptidase, and a combination thereof, and (b) calculating said disease score comprising said level of said at least one biomarker, wherein said disease score represents a disease activity of said chronic obstructive pulmonary disease in said subject.

In some instances, the method further comprises presenting said disease score on a report. In some cases, said disease score is selected from the group consisting of: a numerical value of said disease activity, a categorization of said disease activity above a cutoff, a categorization of said disease activity below a cutoff, a classification of said disease activity into a category, and a combination thereof. In some cases, said disease score identifies said subject as being part of a population, wherein the population is selected from a group consisting of: a population with controlled chronic obstructive pulmonary disease, a population with uncontrolled chronic obstructive pulmonary disease, a population prone to a future acute exacerbation event, a population not prone to a future acute exacerbation event, a population which will benefit from an increased therapy, a population which will benefit from a decreased therapy, and a combination thereof. In some cases, said disease activity is selected from the group consisting of: a measure of exacerbations, a measure of an exacerbation frequency, a measure of an exacerbation severity, a measure of a risk of future exacerbation, a measure of a lung function, a COPD related symptom, a vital sign, a measure of exercise tolerance, a measure of exertion tolerance, a measure of frailty, and a combination thereof.

In some instances, said biological sample is selected from the group consisting of: blood, plasma, serum, dried blood spot, bronchial lavage, nasal swab, saliva, breath condensate, sputum, and a combination thereof. In some cases, said at least one biomarker is selected from the group consisting of: soluble Receptor for Advanced Glycation End products, Platelet Factor 4, P-selectin, Regulated on Activation Normal T Cell Expressed and Secreted (RANTES), Tissue Inhibitor of Metalloproteinase 1, Pulmonary and Activation-Regulated Chemokine, Club cell 16 protein, pro-peptide of atrial natriuretic peptide, Fibrinogen, C-Reactive Protein, Pentraxin 3, Adiponectin, D-Dimer, Interleukin 6, Monocyte chemoattractant protein-1, Cathepsin S, Cystatin C, Serum amyloid A-1, Human Neutrophil Lipocalin, Growth Differentiation Factor 15, Immunoglobulin A, Fibronectin, Alpha-1 Antitrypsin, Chitinase 3-like 1, Pro-calcitonin, Leptin, Immunoglobulin E, Eotaxin, Complement component 1q, soluble ST2, Matrix Metallopeptidase 9, Neutrophil Elastase, Resistin, and a combination thereof.

In some instances, the method further comprises detecting, from said biological sample from said subject, a level of a second biomarker. In some instances, the method further comprises detecting, from said biological sample from said subject, a level of a third biomarker. In some cases, the method further comprises, detecting, from said biological sample from said subject, a level of a fourth biomarker. In some instances, said at least one biomarker is selected from the group consisting of: soluble Receptor for Advanced Glycation End products, Platelet Factor 4, P-selectin, Regulated on Activation Normal T Cell Expressed and Secreted (RANTES), Tissue Inhibitor of Metalloproteinase 1, Pulmonary and Activation-Regulated Chemokine, Club cell 16 protein, pro-peptide of atrial natriuretic peptide, and Fibrinogen. In some cases, at least one biomarker is selected from the group consisting of: C-Reactive Protein, Pentraxin 3, Adiponectin, D-Dimer, Interleukin 6, Monocyte chemoattractant protein-1, Cathepsin S, and Cystatin C. In some cases, said at least one biomarker is selected from the group consisting of:

Serum amyloid A-1, Human Neutrophil Lipocalin, Growth Differentiation Factor 15, Immunoglobulin A, Fibronectin, Alpha-1 Antitrypsin, Chitinase 3-like 1, and Pro-calcitonin. In some cases, said at least one biomarker is selected from the group consisting of: Leptin, Immunoglobulin E, Eotaxin, Complement component 1q, soluble ST2, Matrix Metallopeptidase 9, Neutrophil Elastase, and Resistin. In some cases, said at least one biomarker is a component of a protein complex. In some cases, said at least one biomarker component of said protein complex is selected from the group consisting of: Alpha-1 Antitrypsin, Immunoglobulin A, Complement component 1q, C-Reactive Protein, Pentraxin 3, soluble Receptor for Advanced Glycation End products, High Mobility Group 1, Calprotectin, Platelet Factor 4, Regulated on Activation Normal T Cell Expressed and Secreted (RANTES), Cystatin C, Matrix Metallopeptidase 9, Tissue Inhibitor of Metalloproteinase 1, Chitinase 3-like 1, and any combination thereof.

In some instances, the method further comprises measuring or determining an additional parameter. In some cases, said disease score further comprises said additional parameter. In some cases, said additional parameter is selected from the group consisting of: a pulmonary function test variable, a quantitative computed tomography measure, a score representative of a symptom, a variable representative of an exacerbation history of said subject, a variable representative of a demographic of said subject, a variable representative of a risk factor of said subject, a variable representative of us of a medication of said subject, a variable representative of a comorbid condition of said subject, and any combination thereof. In some cases, said pulmonary function test variable is selected from the group consisting of: a ratio of forced expiratory volume in 1 second (FEV1) to a forced vital capacity (FVC), FEV1 in liters, FVC in liters, FEV1 in percent predicted value, FEV1 reversibility, residual volume/total lung capacity ratio, and any combination thereof. In some cases, said quantitative computed tomography measure is selected from the group consisting of: Low Attenuation Area at max inspiration, Low Attenuation Area at max expiration, airway wall area, airway wall thickness, a parametric measure of emphysema or small airway disease, and any combination thereof. In some cases, said symptom is selected from the group consisting of: dyspnea, dyspnea on exertion, dyspnea on performing daily activities, cough, phlegm production, chest tightness, sleep quality, energy level, confidence level, and any combination thereof. In some cases, said exacerbation history is selected from the group consisting of: an exacerbation occurrence in a given time frame, a form of setting of care received, a care received, and any combination thereof. In some cases, said demographic is selected from the group consisting of: age, sex, race, and any combination thereof. In some cases, said risk factor is selected from the group consisting of: smoking, smoking exposure, activity level, body mass, body mass index, and any combination thereof. In some cases, said medication is selected from the group consisting of: a steroid, a long-acting beta-agonist, a long-acting muscarinic antagonist, a phosphodiesterase inhibitor, an anti-inflammatory, an antibiotic, a supplement, and any combination thereof. In some cases, said comorbid condition is selected from the group consisting of: a metabolic disorder, a vascular disorder, a circulatory disorder, a cardiac disorder, a non-chronic obstructive pulmonary disease lung disorder, a liver disorder, a gastrointestinal disorder, a central nervous system disorder, and any combination thereof. In some instances, the method further comprises administering a treatment to said subject based on said disease score. In some cases, said treatment is selected from Table 1.

In some instances, said detecting comprises performing a plurality of assays on said biological sample. In some cases, said plurality of assays are selected from the group consisting of: enzyme-linked immunosorbent assay, homogeneous immunoassay, fluorescence immunoassay, chemiluminescence immunoassay, electro-chemiluminescence immunoassay, fluorescence resonance energy transfer immunoassay, time resolved fluorescence immunoassay, lateral flow immunoassay, microspot immunoassay, surface plasmon resonance assay, ligand assay, a clotting assay, immunocapture coupled with mass spectrometry, a non-optical immunoassay, and any combination thereof. In some cases, said non-optical immunoassay is an acoustic membrane microparticle (AMMP) assay. In some cases, said detecting said level said at least one biomarker comprises contacting said biological sample with at least one antibody with specific binding for said at least one biomarker. In some cases, said detecting comprises detecting binding of said at least one antibody to said at least one biomarker. In some cases, said at least one antibody is a monoclonal antibody. In some cases, said at least one antibody is a polyclonal antibody. In some cases, said at least one antibody is bound to a solid support. In some cases, said biological sample is not diluted prior to said detecting.

In some instances, the method further comprises detecting, from at least a second biological sample from said subject, a second level of said at least one biomarker. In some cases, said calculating said disease score comprises comparing said level of said at least one biomarker from said biological sample with said second level of said at least one biomarker from said second biological sample.

Disclosed herein, in certain embodiments, are methods of determining a disease status of a subject having, suspected of having, or is at risk of progressing to chronic obstructive pulmonary disease (COPD), the method comprising: a) detecting, from at least a first biological sample from said subject, a level of four or more biomarkers, wherein said four or more biomarkers are selected from the following classes of biomarkers: a platelet degranulation product, a cathepsin, an endopeptidase, an endopeptidase inhibitor, a cystatin, a serpin, an immunoglobulin, a coagulation protein, a fibrosis or fibrinolysis protein, a fibrin degradation product, a protein involved in platelet activity, a chemotaxis protein, a chemokine produced by an immune response, an interleukin receptor or receptor-like protein, a Toll-like receptor or protein with Toll-like receptor domains, a complement pathway protein, a leukocyte related protein, an adipokine, an adipose-derived hormone, a protein involved in the insulin pathway, a protein involved with insulin resistance, a protein involved with calcium homeostasis, an acute phase protein, a pentraxin, a natriuretic peptide, a lipoprotein, an advanced glycation end-product, an extracellular glycoprotein, an apolipoprotein, a chitinase, a protein from the transforming growth factor beta superfamily, and a club cell related protein; b) calculating a disease score by combining said level of said four or more biomarkers, wherein said disease score is indicative of a disease status of said subject; and c) presenting said disease score on a report.

In some embodiments, said calculating comprises logarithm transformation of a level of one or more biomarkers. In some embodiments, at least one biomarker level is incorporated in at least one term in the disease score calculation with a negative exponent of the biomarker level or negative coefficient multiplying a logarithm transformation of the biomarker level. In some embodiments, said calculating comprises combining logarithm transformed levels of said four or more biomarkers. In some embodiments, said calculating comprises comparing a combination of one or more biomarker levels in an ensemble of classification trees for performing subsequent calculations of three or more biomarker levels.

In some embodiments, said four or more biomarkers are selected from Table 1 of the specification. In some embodiments, said four or more biomarkers are selected from those indicated in examples 1 through 5 in the specification. In some embodiments, said four or more biomarkers comprise at least four biomarkers selected from the group consisting of: HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, NT-proBNP, ANP, NT-proANP, cTnI, Cystatin C, D-Dimer, Resistin, Insulin, GDF-15, and CC16. In some embodiments, said four or more biomarkers comprise at least four biomarkers selected from the group consisting of: HNL, Leptin, sRAGE, Eotaxin1, MMP-9, TIMP1, SAA1, CRP, C1q, Neutrophil Elastase, P-selectin, A1AT, IgE, IgA, YKL-40, GDF-15, NT-proANP, NT-proBNP, sST2, Fibronectin, Adiponectin, and Resistin. In some embodiments, said four or more biomarkers includes at least two biomarkers selected from the group consisting of: sRAGE, HNL, C1q, Leptin, GDF-15, IgA and Eotaxin1.

In some embodiments, said four or more biomarkers includes at least one complex of two or more biomarkers. In some embodiments, at least one complex of biomarkers includes at least one biomarker selected from the group consisting of: PF4, RANTES, A1AT, Nuetrophil elastase, IgA, IgE, C1q, CRP, sRAGE, IL1beta, HMGB1, Calprotectin, ST2, IL33, Eotaxin1, Fibronectin, and HNL.

In some embodiments, detecting a level of four or more biomarkers comprises performing a plurality of assays on the at least a first biological sample. In some embodiments, the plurality of assays comprises two or more assays. In some embodiments, at least one of the plurality of assays is selected from the group consisting of: ELISA, homogeneous immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, a clotting assay, and immunocapture coupled with mass spectrometry. In some embodiments, at least one of the plurality of assays comprises an assay that does not use photometric or radiometric transduction. In some embodiments, at least one of the plurality of assays is a non-optical immunoassay. In some embodiments, the non-optical immunoassay is an acoustic membrane microparticle (AMMP) assay.

In some embodiments, the method further comprises comparing said disease score to a predetermined cutoff or reference value associated with out of control, unstable or acute COPD events. In some embodiments, said disease score further comprises at least one Computed Tomography (CT)-derived parameter. In some embodiments, said disease score further comprises at least one lung function-derived parameter. In some embodiments, said disease score further comprises at least one spirometry-derived parameter. In some embodiments, said disease score further comprises one or more clinical parameters, one or more risk factors of said subject, or both. In some embodiments, said one or more clinical parameters comprises age, race, gender, blood pressure, temperature, weight, height, body mass index, anthropometric measurements, strength, exercise tolerance, estimated blood volume or a combination thereof. In some embodiments, said one or more clinical parameters is a parameter selected from the group consisting of: disease classification by Global Initiative for Chronic Obstructive Lung Disease (GOLD) guidelines, spirometry parameters, symptoms assessed by COPD Assessment Test score, symptoms assessed by modified Medical Research Council score, COPD exacerbations counted as presentation of acute worsening of respiratory symptoms that is treated, COPD exacerbations counted as presentation of acute worsening of respiratory symptoms by physician's classification, symptoms assessed by modified Borg Scale, symptoms assessed by Baseline or Transition Dyspnea Indices, symptoms assessed by UCSD shortness of breath questionnaire, symptoms assessed by American Heart Association Dyspnea Index, symptoms assessed by Saint Georges Respiratory Questionnaire, and any combination thereof. In some embodiments, said one or more clinical parameters comprises one or more imaging parameters. In some embodiments, said one or more imaging parameters comprises one or more CT images. In some embodiments, said one or more CT images comprises Low Attenuation Area at max inspiration, Low Attenuation Area at max expiration, airway wall area, airway wall thickness, parametric measures of emphysema or small airway disease, or any combination thereof. In some embodiments, said one or more risk factors comprises smoking status and history of said subject, co-morbid conditions and associated treatments, or any combination thereof. In some embodiments, said co-morbid conditions and associated treatments comprises hypertension and blood pressure lowering medications; cardiovascular disease and statin and ACE medications; diabetes and TZD and metformin medications; GERD and protein pump inhibitors; anxiety and depression and associated assessments and medications; or any combination thereof.

In some embodiments, the method further comprises administering a treatment to said subject based on said disease score. In some embodiments, said treatment is selected from Table 1. In some embodiments, said at least first biological sample comprises blood, plasma, serum, dried blood spot, bronchial lavage, nasal swab, saliva, breath condensate, or sputum. In some embodiments, said at least first biological sample is obtained during admitted stay in hospital for an exacerbation. In some embodiments, said at least first biological sample is obtained during evaluation in a hospital emergency department. In some embodiments, at least first biological sample is obtained 1-90 days after discharge from hospital or emergency department. In some embodiments, said at least first biological sample is obtained 3-30 days after discharge from hospital or emergency department. In some embodiments, said at least first biological sample is obtained 5-21 days after discharge from hospital or emergency department.

Disclosed herein, in at least some embodiments, are methods of monitoring disease status in a subject suffering or suspected to be suffering from chronic obstructive pulmonary disease (COPD), and/or associated disease mechanisms, the method comprising: a) detecting, from at least a first biological sample from said subject at a first time point, a level of four or more biomarkers, wherein said four or more biomarkers are selected from the following classes of biomarkers: a platelet degranulation product, a cathepsin, an endopeptidase, an endopeptidase inhibitor, a cystatin, a serpin, an immunoglobulin, a coagulation protein, a fibrosis or fibrinolysis protein, a fibrin degradation product, a protein involved in platelet activity, a chemotaxis protein, a chemokine produced by an immune response, an interleukin receptor or receptor-like protein, a Toll-like receptor or protein with Toll-like receptor domains, a complement pathway protein, a leukocyte related protein, an adipokine, an adipose-derived hormone, a protein involved in the insulin pathway, a protein involved with insulin resistance, a protein involved with calcium homeostasis, an acute phase protein, a pentraxin, a natriuretic peptide, a lipoprotein, an advanced glycation end-product, an extracellular glycoprotein, an apolipoprotein, a chitinase, a protein from the transforming growth factor beta superfamily, and a club cell related protein; b) detecting, from at least a second biological sample taken from said subject at a second time point, the level of the four or more biomarkers detected in the at least first biological sample; c) calculating a first and second disease score, wherein calculating said first disease score comprises combining said level of four or more biomarkers at said first time point, and wherein calculating said second disease score comprises combining said level of four or more biomarkers at said second time point, wherein said first and second disease scores are indicative of a disease status of said subject; and d) identifying a trend of said first and second disease scores from said first time point to said second time point, wherein if said trend of said first and second disease scores are identified as increasing, said subject is identified as uncontrolled, unstable, relapsing, recurring or being at increased risk for a future disease related event, and if said trend of said first and second disease scores are identified as decreasing, said subject is identified as under control, stable, recovering, or being at lower risk for a future disease related event; and e) presenting said trend on a report.

In some embodiments, calculating the first disease score comprises calculating a first predetermined combination of the level of four or more biomarkers present in the at least a first biological sample at the first time point, and wherein the calculating the second disease score comprises calculating a second predetermined combination of the level of four or more biomarkers present in the at least a second biological sample at the second time point. In some embodiments, calculating the predetermined combination includes at least one term in the calculation with a negative exponent of at least one biomarker level or a negative coefficient multiplying a logarithm transformation of at least one biomarker level. In some embodiments, calculating the predetermined combination includes relationships between logarithm transformed levels of four or more biomarkers. In some embodiments, calculating the predetermined combination includes calculating a classification tree, with two or more branches, where branched calculations of mathematical relationships between three or more biomarkers are gated by mathematical relationships of at least one of the four or more biomarkers. In some embodiments, the mathematical relationships include logarithm transformed levels of at least one of the four or more biomarkers.

In some embodiments, said four or more biomarkers are selected from Table 1 of the specification. In some embodiments, said four or more biomarkers are selected from those indicated in examples 1 through 8 in the specification. In some embodiments, said four or more biomarkers comprise at least four biomarkers selected from the group consisting of: HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, NT-proBNP, ANP, NT-proANP, cTnI, Cystatin C, D-Dimer, Resistin, Insulin, GDF-15, and CC16. In some embodiments, said four or more biomarkers comprise at least four biomarkers selected from the group consisting of: HNL, Leptin, sRAGE, Eotaxin1, MMP-9, TIMP1, SAA1, CRP, C1q, Neutrophil Elastase, P-selectin, A1AT, IgE, IgA, YKL-40, GDF-15, NT-proANP, NT-proBNP, sST2, Fibronectin, Adiponectin, and Resistin. In some embodiments, said four or more biomarkers comprise at least two biomarkers selected from the group consisting of: sRAGE, HNL, C1q, Leptin, GDF-15, IgA, and Eotaxin1. In some embodiments, said four or more biomarkers includes at least one complex of two or more biomarkers. In some embodiments, at least one complex of biomarkers includes at least one biomarker selected from the group consisting of: PF4, RANTES, A1AT, Neutrophil elastase, IgA, IgE, C1q, CRP, sRAGE, IL1beta, HMGB1, Calprotectin, ST2, IL33, Eotaxin1, Fibronectin, and HNL.

In some embodiments, detecting a level of four or more biomarkers comprises performing a plurality of assays on the at least a first biological sample. In some embodiments, the plurality of assays comprises at least two assays. In some embodiments, at least one plurality of assays is selected from the group consisting of: ELISA, homogeneous immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance immunoassay, ligand assay, clotting assay, and immunocapture coupled with mass spectrometry. In some embodiments, at least one of said plurality of assays comprises an immunoassay that does not use photometric or radiometric transduction. In some embodiments, at least one of the plurality of assays is a non-optical immunoassay. In some embodiments, the non-optical immunoassay is an acoustic membrane microparticle (AMMP) assay.

In some embodiments, the method further comprises comparing said first and second disease scores to a predetermined cutoff or reference value associated with an increased risk of unstable or acute COPD events. In some embodiments, said first and second disease scores further comprise at least one Computed Tomography (CT)-derived parameter. In some embodiments, said disease score further comprises at least one lung function-derived parameter. In some embodiments, said disease score further comprises at least one spirometry-derived parameter.

In some embodiments, said first and second disease scores further comprise one or more clinical parameters, one or more risk factors of said subject, or both. In some embodiments, said one or more clinical parameters comprises age, race, gender, blood pressure, temperature, weight, height, body mass index, anthropometric measurements, strength, exercise tolerance, estimated blood volume or a combination thereof. In some embodiments, said one or more clinical parameters comprises disease classification by Global Initiative for Chronic Obstructive Lung Disease (GOLD) guidelines, spirometry parameters, symptoms assessed by COPD Assessment Test score, symptoms assessed by modified Medical Research Council score, COPD exacerbations counted as presentation of acute worsening of respiratory symptoms that is treated, COPD exacerbations counted as presentation of acute worsening of respiratory symptoms by physician's classification, symptoms assessed by modified Borg Scale, symptoms assessed by Baseline or Transition Dyspnea Indices, symptoms assessed by UCSD shortness of breath questionnaire, symptoms assessed by American Heart Association Dyspnea Index, symptoms assessed by Saint Georges Respiratory Questionnaire or any combination thereof. In some embodiments, said one or more clinical parameters comprises one or more imaging parameters. In some embodiments, said one or more imaging parameters comprises one or more CT images. In some embodiments, said one or more CT images comprises Low Attenuation Area at max inspiration, Low Attenuation Area at max expiration, airway wall area, airway wall thickness, parametric measures of emphysema or small airway disease, or any combination thereof. In some embodiments, said one or more risk factors comprises smoking status and history of said subject, co-morbid conditions and associated treatments, or any combination thereof. In some embodiments, said co-morbid conditions and associated treatments comprises hypertension and blood pressure lowering medications; cardiovascular disease and statin and ACE medications; diabetes and TZD and metformin medications; GERD and protein pump inhibitors; anxiety and depression and associated assessments and medications; or any combination thereof.

In some embodiments, the method further comprises administering a treatment to said subject based on said trend. In some embodiments, said treatment is selected from Table 1. In some embodiments, said at least a first biological sample comprises blood, plasma, serum, dried blood spot, bronchial lavage, nasal swab, saliva, breath condensate or sputum. In some embodiments, at least one time point of said first or second time point comprises a time point after said subject has been treated with a COPD therapy. In some embodiments, said trend indicates said COPD therapy should be halted, said COPD therapy should be prolonged, or said COPD therapy should be altered. In some embodiments, said at least one time point comprises 3-90 days after said subject has been treated with said COPD therapy. In some embodiments, said at least one time point comprises 3-14 days after said subject has been treated with said COPD therapy. In some embodiments, said at least one time point comprises 14-36 days after said subject has been treated with said COPD therapy. In some embodiments, said at least one time point comprises 36-90 days after said subject has been treated with said COPD therapy. In some embodiments, said COPD therapy is selected from the group consisting of: an antibiotic, a steroid, a dilator, an anti-coagulant, a blood thinner, a transfusion of whole or processed blood components, a bronchodilator, a muscarinic antagonist, an anti-inflammatory, a targeted anti-inflammatory, mechanically assisted ventilation, oxygen assistance and any combination thereof.

Disclosed herein, in certain embodiments, are methods of detecting a biomarker signature of a subject having, suspected of having, or is at risk of progressing to chronic obstructive pulmonary disease (COPD), the method comprising: a) obtaining a biological sample from said subject; and b) detecting a level of four or more biomarkers comprising the biomarker signature by performing a plurality of assays on the biological sample, wherein at least one of the plurality of assays is a non-optical immunoassay; and wherein said four or more biomarkers are selected from the following classes of biomarkers: a platelet degranulation product, a cathepsin, an endopeptidase, an endopeptidase inhibitor, a cystatin, a serpin, an immunoglobulin, a coagulation protein, a fibrosis or fibrinolysis protein, a fibrin degradation product, a protein involved in platelet activity, a chemotaxis protein, a chemokine produced by an immune response, an interleukin receptor or receptor-like protein, a Toll-like receptor or protein with Toll-like receptor domains, a complement pathway protein, a leukocyte related protein, an adipokine, an adipose-derived hormone, a protein involved in the insulin pathway, a protein involved with insulin resistance, a protein involved with calcium homeostasis, an acute phase protein, a pentraxin, a natriuretic peptide, a lipoprotein, an advanced glycation end-product, an extracellular glycoprotein, an apolipoprotein, a chitinase, a protein from the transforming growth factor beta superfamily, and a club cell related protein. In some embodiments, said four or more biomarkers includes at least one complex of two or more biomarkers.

In some embodiments, the plurality of assays comprises at least two assays. In some embodiments, each of the plurality of assays are selected from the group consisting of: ELISA, non-optical immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, and immunocapture coupled with mass spectrometry.

Disclosed herein, in certain embodiments, are methods for generating quantitative data for a subject, the method comprising: a) obtaining a biological sample from the subject; and b) performing a plurality of immunoassays on said biological sample to generate a dataset comprising the quantitative data, wherein the quantitative data represents levels of at least four or more biomarkers selected from group consisting of: HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, NT-proBNP, ANP, NT-proANP, cTnI, Cystatin C, D-Dimer, Resistin, Insulin, GDF-15, CC16; and wherein the subject has or is suspected of having COPD. In some embodiments, said four or more biomarkers includes at least one complex of two or more biomarkers. In some embodiments, the plurality of immunoassays comprises at least two assays. In some embodiments, each of the plurality of assays are selected from the group consisting of: ELISA, non-optical immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, and immunocapture coupled with mass spectrometry.

Disclosed herein, in certain embodiments, are methods of detecting a biomarker signature of a subject suffering or suspected to be suffering from chronic obstructive pulmonary disease (COPD), and/or associated disease mechanisms, the method comprising: a) obtaining a biological sample from said subject; and b) detecting a level of four or more biomarkers comprising the biomarker signature by performing a plurality of assays on the biological sample, wherein at least one of the plurality of assays is a non-optical immunoassay; and wherein said four or more biomarkers are selected from the group consisting of: HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, NT-proBNP, ANP, NT-proANP, cTnI, Cystatin C, D-Dimer, Resistin, Insulin, GDF-15, and CC16. In some embodiments, said four or more biomarkers includes at least one complex of two or more biomarkers. In some embodiments, the plurality of assays comprises at least two assays. In some embodiments, each of the plurality of assays are selected from the group consisting of: ELISA, non-optical immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, and immunocapture coupled with mass spectrometry.

Disclosed herein, in certain embodiments, are methods of detecting a biomarker signature of a subject having or suspected of having chronic obstructive pulmonary disease (COPD), the method comprising: a) obtaining a biological sample from said subject; and b) detecting a level of a first biomarker comprising a biomarker signature by performing a non-optical immunoassay on the biological sample; and wherein said first biomarker is a first biomarker selected from a biomarker class comprising: a platelet degranulation product, a cathepsin, an endopeptidase, an endopeptidase inhibitor, a cystatin, a serpin, an immunoglobulin, a coagulation protein, a fibrosis or fibrinolysis protein, a fibrin degradation product, a protein involved in platelet activity, a chemotaxis protein, a chemokine produced by an immune response, an interleukin receptor or receptor-like protein, a Toll-like receptor or protein with Toll-like receptor domains, a complement pathway protein, a leukocyte related protein, an adipokine, an adipose-derived hormone, a protein involved in the insulin pathway, a protein involved with insulin resistance, a protein involved with calcium homeostasis, an acute phase protein, a pentraxin, a natriuretic peptide, a lipoprotein, an advanced glycation end-product, an extracellular glycoprotein, an apolipoprotein, a chitinase, a protein from the transforming growth factor beta superfamily, and a club cell related protein. In some embodiments, the method further comprises detecting a level of a second biomarker comprising the biomarker signature by performing a second assay. In some embodiments, the second assay is selected from the group consisting of: ELISA, homogeneous immunoassay, non-optical, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, clotting assay, and immunocapture coupled with mass spectrometry. In some embodiments, the method further comprises detecting a level of a third biomarker comprising the biomarker signature by performing a third assay. In some embodiments, the third assay is selected from the group consisting of: ELISA, homogeneous immunoassay, non-optical, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, clotting assay, and immunocapture coupled with mass spectrometry. In some embodiments, the method further comprises detecting a level of a fourth biomarker comprising the biomarker signature by performing a fourth assay. In some embodiments, the fourth assay is selected from the group consisting of: ELISA, homogeneous immunoassay, non-optical, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, clotting assay, and immunocapture coupled with mass spectrometry. In some embodiments, said four or more biomarkers includes at least one complex of two or more biomarkers.

Disclosed herein, in certain embodiments, are methods of detecting a biomarker signature of a subject having or suspected of having chronic obstructive pulmonary disease (COPD), the method comprising: a) obtaining a biological sample from said subject; and b) detecting a level of four or more biomarkers comprising the biomarker signature by performing a plurality of assays on the biological sample, and wherein said four or more biomarkers are selected from the group consisting of: HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, NT-proBNP, ANP, NT-proANP, cTnI, Cystatin C, D-Dimer, Resistin, Insulin, GDF-15, and CC16. In some embodiments, said four or more biomarkers includes at least one complex of two or more biomarkers. In some embodiments, the plurality of assays comprises at least two assays. In some embodiments, each of the plurality of assays are selected from the group consisting of: ELISA, non-optical immunoassay, fluorescence immunoassay, chemiluminescence immunoassay, electro-chemiluminescence immunoassay, fluorescence resonance energy transfer (FRET) immunoassay, time resolved fluorescence immunoassay, lateral flow immunoassay, microspot immunoassay, surface plasmon resonance assay, ligand assay, and immunocapture coupled with mass spectrometry.

In some embodiments said four or more biomarkers comprise at least four of sRAGE, PARC, Leptin, RANTES, IgA, C1q, IL-6. In some embodiments, said four or more biomarkers comprise at least four of sRAGE, IL-6, Leptin, HNL, Adiponectin and a quantitative CT measure of small airway disease. In some embodiments, said four or more biomarkers comprise at least four of HNL, Leptin, IgE, YKL-40, P-selectin, IgA, TIMP-1, SAA1, IL-6, and age. In some embodiments said four or more biomarkers comprise at least HNL, IgE, Leptin and age. In some embodiments said four or more biomarkers comprise at least four of HNL, PCT, PF4, P-selectin, IgE, IL-6, Eotaxin1, SAA1, PARC, TIMP-1, IgA, sRAGE. In some embodiments said four or more biomarkers comprise at least four of IgE, PCT, PF4, P-selectin, HNL, Eotaxin1, PARC, IL-6. In some embodiments said four or more biomarkers comprise at least four of HNL, PF4, P-selectin, PCT, IgE, SAA1, sRAGE, PARC, IL-6, IgA, IgA, TIMP-1 and a symptoms score (e.g. COPD Assessment Test—CAT—score, Saint Georges Respiratory Questionnaire—SGRQ—symptoms score). In some embodiments said four or more biomarkers comprise at least four of IL-6, MMP-9, IgA, PCT, IgE, HNL, PARC and gender. In some embodiments said four or more biomarkers comprise at least four of MMP-9, SAA1, PF4, P-selectin, HNL, sRAGE, TIMP-1, CRP, YKL-40 and gender.

In some embodiments said four or more biomarkers comprise at least four of SAA1, Eotaxin1, C1q, NT-ProANP, IL-6, GDF-15, IgE, IgA, sRAGE. In some embodiments said four or more biomarkers comprise at least four of Leptin, GDF-15, IgE, TIMP-1, MMP-9, Eotaxin, NT-proANP and gender. In some embodiments said four or more biomarkers comprise at least four of SAA1, Adiponectin, C1q, IL-6, Eotaxin and gender. In some embodiments said four or more biomarkers comprise at least four of SAA1, IgE, Eotaxin, NT-proANP, GDF-15. IL-6, IgA, C1q, TIMP-1, Adiponectin and a symptoms score (e.g. CAT or SGRQ score). In some embodiments said four or more biomarkers comprise at least four of GDF-15, IgE, Leptin, MMP-9, NT-proANP, TIMP-1, a symptoms score (eg. CAT or SGRQ) and gender. In some embodiments said four or more biomarkers comprise at least four of Adiponectin, SAA1, NT-proANP, P-selectin, IL-6, Eotaxin, symptoms score (eg. CAT) and gender.

In some embodiments said four or more biomarkers comprise at least four of sRAGE, Eotaxin1, C1q, HNL, IgE, A1AT, TIMP-1, MMP-9, D-Dimer. In some embodiments said four or more biomarkers comprise at least one of gender, history of 2 or more treated exacerbations in past 12 months, symptoms score (CAT or SGRQ scores), inhaled steroids use, forced expiratory volume in 1 second, smoking and age. In some embodiments said four or more biomarkers comprise at least four of C1q, HNL, Eotaxin1, sRAGE, Cathepsin S, Resistin, IgE, YKL-40, PF4, Neutrophil Elastase, A1AT, P-selectin, MCP-1, and symptoms score (e.g. CAT or SGRQ scores). In some embodiments said four or more biomarkers comprise at least four of C1q, sRAGE, Eotaxin1, Resistin, HNL, A1AT, YKL-40, IgE, Cathepsin S, Neutrophil Elastase, RANTES, PF4, P-selectin and symptoms score (e.g. CAT or SGRQ scores).

In some embodiments, said four or more biomarkers comprise at least four of HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, and IgA. In some embodiments, said four or more biomarkers comprise at least three of HNL, CRP, sRAGE, SAA1, Fibrinogen, Leptin, Eotaxin1, YKL-40, PCT, RANTES, PF4, P-selectin, A1AT, sST2, and NT-proBNP. In some embodiments, said four or more biomarkers comprise at least two of sRAGE, SAA1, Leptin, Eotaxin1, YKL-40, PCT, sST2, and NT-proBNP. In some embodiments, said four or more biomarkers comprise at least two of YKL-40, sRAGE, PCT, MCP-1, IL6 and sST2. In some embodiments, said four or more biomarkers comprise at least two of Leptin, Eotaxin1, Adiponectin, MCP-1, SAA1 and IgE. In some embodiments, said four or more biomarkers comprise at least two of PF4, P-selectin, RANTES, Fibrinogen and Fibronectin. In some embodiments, said four or more biomarkers comprise at least two of sST2, CRP, Eotaxin1, Fibronectin, MCP-1, and SAA1. In some embodiments, said four or more biomarkers comprise at least two of HNL, sRAGE, MMP-9, TIMP1, CRP and IgA. In some embodiments, said four or more biomarkers includes at least one of sRAGE, HNL, C1q, Leptin, GDF-15, IgA and Eotaxin1 sST2.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a non-optical, acoustic immunoassay amenable to performing the methods described herein. FIG. 1B depicts an example of PF4-RANTES complexes measured as an assembly from individual recombinant proteins, combined at a high protein concentration to favor molecular complex formation. Data shown is for a titration of PF4-RANTES molecular complexes.

FIG. 2 depicts sputum Interleukin-8 (IL8) levels in an Alpha-1 Antitrypsin Deficient (A1AD)/COPD-exacerbating cohort as measured by an ELISA assay.

FIG. 3A depicts normalized PF4-RANTES/Alpha-1 Antitrypsin (A1AT) assay data, where levels are recovered from a reference standard constructed from a titrated mix of recombinant components, collected from the sputum of an A1AD/COPD-exacerbating cohort. FIG. 3B depicts normalized PF4-RANTES/A1AT assay data collected from sputum of an A1AD/COPD-exacerbating cohort.

FIG. 4 depicts normalized PF4-RANTES/A1AT assay data collected from sputum of an A1AD/COPD-exacerbating cohort days 0-10 indexed from admission.

FIG. 5 depicts normalized PF4-RANTES/A1AT assay data collected from sputum of an A1AD/COPD-exacerbating cohort days 5-30 indexed from admission.

FIG. 6 depicts normalized PF4-RANTES/A1AT assay data measured longitudinally in sputum from A1AD/COPD-exacerbating cohort versus days from admission.

FIG. 7 depicts median and interquartile ranges plotted for a combination of CRP, MMP-9/TIMP1, IgA/TIMP1, SAA1, and PF4 multiplied by RANTES levels (PF4×RANTES), measured in non-COPD and mild/moderate COPD cohorts.

FIG. 8 depicts median and interquartile ranges plotted for a combination of CRP, MMP-9/TIMP1, IgA/TIMP1, SAA1, and PF4 multiplied by RANTES (PF4×RANTES) levels with CT low area attenuation measured in non-COPD and mild/moderate COPD cohorts.

FIG. 9 depicts combined molecular markers CRP, MMP-9/TIMP1, IgA/TIMP1, SAA1, and PF4 multiplied by RANTES levels (PF4×RANTES) plotted versus lung function FEV1/FVC in non-COPD and mild/moderate COPD cohorts.

FIG. 10 depicts combined molecular markers CRP, MMP-9/TIMP1, IgA/TIMP1, SAA1, and PF4 multiplied by RANTES levels (PF4×RANTES) with CT low area attenuation plotted versus lung function FEV1/FVC in non-COPD and mild/moderate COPD cohorts.

FIG. 11 depicts median and interquartile ranges plotted for a combination of IgA, Adiponectin, and PF4 multiplied by RANTES levels (PF4×RANTES) measured in non-COPD and mild/moderate COPD cohorts.

FIG. 12 depicts median and interquartile ranges plotted for a combination of IgA, Adiponectin, and PF4 multiplied by RANTES levels (PF4×RANTES) with CT low area attenuation measured in non-COPD and mild/moderate COPD cohorts.

FIG. 13 depicts combined molecular markers IgA, Adiponectin, and PF4 multiplied by RANTES levels (PF4×RANTES) plotted versus lung function FEV1/FVC in non-COPD and mild/moderate COPD cohorts.

FIG. 14 depicts combined molecular markers IgA, Adiponectin, and PF4 multiplied by RANTES levels (PF4×RANTES) with CT low area attenuation plotted versus lung function FEV1/FVC in non-COPD and mild/moderate COPD cohorts.

FIG. 15 depicts combined molecular markers PF4, MMP-9/TIMP1, C1q, and C3a measured in COPD, non-COPD never smoked and non-COPD with smoking history cohorts correlated with lung function FEV1% predicted.

FIG. 16 depicts combined molecular markers PF4, MMP-9/TIMP1, C1q, and C3a, with CT low attenuation area measured in COPD, non-COPD never smoked and non-COPD with smoking history cohorts correlated with lung function FEV1% predicted.

FIG. 17 depicts combined molecular markers PF4, MMP-9/TIMP1, C1q, and 1/Adiponectin measured in COPD and non-COPD with smoking history cohorts correlated with lung function FEV1% predicted.

FIG. 18 depicts combined molecular markers PF4, MMP-9/TIMP1, C1q, and 1/Adiponectin with CT low area attenuation measured in COPD and non-COPD with smoking history cohorts correlated with lung function FEV1% predicted.

FIG. 19 depicts blood biomarker combination prediction Receiver Operating Characteristic (ROC) curve for COPD diagnosis versus controls. Training of the biomarker combination algorithm was performed on approximately 268 diagnosed COPD subjects and 100 controls. COPD diagnosed subjects include stages I through IV of the disease. Controls include a similar age range of asthmatics, obstructive sleep apnea and common co-morbidity diagnosed patients, which are known diseases and disorders that overlap with COPD. The biomarker combination shown includes sRAGE, TIMP-1, Leptin, Adiponectin, Fibronectin, YKL-40, IgE, Eotaxin, P-Selectin, PF4, MCP-1, CRP, SAA1, PCT, MMP-9, IgA, and HNL.

FIG. 20 depicts blood biomarker combination prediction of COPD patient FEV1% predicted values, recorded in the patient medical histories in the prior 12 months, continuous scale. Included in the model shown are combinations of log transformed levels of Fibrinogen, CRP, HNL, fibronectin, MMP-9, IgA, MCP-1, sRAGE, PCT, IgE, Adiponectin, P-selectin, Leptin, SAA1, TIMP-1.

FIGS. 21A-21B depicts blood biomarker combination prediction ROC curve for COPD Assessment Test (CAT) scores. FIG. 21A shows a model prediction for groups that include both COPD diagnosed and controls separated by level, <10 versus >=10 on a scale of 40. Of 368 total subjects, 286 have scores >=10 while 82 have scores <10. The model trained to predict this grouping is a combination of levels of sRAGE, Eotaxin, HNL, IL6, PF4, YKL-40, SAA1, and RANTES.

FIG. 21B shows a separately trained model of 293 total subjects, including both COPD and controls, 231 having scores >=10 with 62 having scores <10. This model includes a combination of HNL, PF4, sRAGE, CRP, MMP-9, IgA, Eotaxin and MCP-1.

FIG. 22 depicts blood biomarker combination prediction ROC curve for modified Medical Research Council (mMRC) Dyspnea scores for 255 COPD diagnosed subjects. The combination of biomarkers depicted is Fibrinogen, PF4, Eotaxin, SAA1, YKL-40, Leptin, sRAGE, IgA, and PCT.

FIGS. 23A-23B illustrate modified Medical Research Council (mMRC) Dyspnea scores for 414 COPD diagnosed subjects using the following combination of biomarkers: Eotaxin1, PF4, sRAGE, Leptin, HNL, PARC, CRP, and MCP-1. FIG. 23A depicts probability scores versus mMRC clinical grouping. FIG. 23B depicts the associated probability densities per clinical grouping. While the clinical grouping separation is not strong with many shared in the middle mode of probability density, each group does show uniquely separated low (<0.4) and high (>0.6) probability modes respectively. Both may have value for negative predictive value and positive predictive value for worse future outcomes. For example, recently chronically elevated dyspnea persistent in the presence of increasing COPD treatments has been identified in a class of COPD patients with worse outcomes.

FIGS. 24A-24B depicts blood biomarker combination prediction ROC curve for COPD Exacerbations History, reported in the prior 12 months. Four hundred and eight COPD diagnosed subjects were included in the analytical model training. One hundred and seventy-four of those had reported a COPD exacerbation (acute event) within the past 12 months. Sixty-one recorded two or more. Algorithms were constructed for <2 versus 2 or more reported exacerbations. The combination of biomarkers giving results for frequent exacerbators shown in FIG. 24A is SAA1, Eotaxin, IgA, MCP-1, Adiponectin, TIMP-1, sRAGE, IgE, PF4, Leptin, RANTES, and YKL-40. The combination of biomarkers giving results for any exacerbation in the recent past, shown in FIG. 24B, is Adiponectin, sRAGE, Eotaxin, P-selectin, TIMP-1, Leptin, SAA1, YKL-40, and MCP-1.

FIG. 25 shows algorithm performances predictive events in a prospective cohort. The cohort comprised of 104 subjects, each with 12month history of >=1 exacerbations were followed up with a mean of 100 days over winter months subsequent to baseline blood draws. Thirty-four follow up exacerbation events were recorded. An overall positive rate of exacerbations of 0.33 was observed (negative rate 0.67). Algorithm performances for predicting the future events from baseline blood analysis are: area under the curve (AUC) of 0.69 for biomarkers and CAT score (model comprising SAA1, IgE, Eotaxin, NT-proANP, GDF-15. IL-6, IgA, C1q, TIMP-1, Adiponectin and CAT score), AUC of 0.72 for biomarkers only (model comprising SAA1, Eotaxin1, C1q, NT-ProANP, IL-6, GDF-15, IgE, IgA, and sRAGE) and AUC of 0.75 for the extended biomarkers model (comprising SAA1, Eotaxin1, C1q, NT-ProANP, IL-6, GDF-15, IgE, IgA, sRAGE, and including high CRP and high and low YKL-40 subjects).

FIG. 26 depicts blood biomarker combination prediction ROC curve for COPD Exacerbations History requiring hospitalization. Of the two hundred and sixty-seven subjects, thirty three reported an exacerbation requiring hospitalization. An algorithm was constructed for <1 versus 1 or more reported hospitalizations. The combination of markers giving the results shown is sRAGE, SAA1, YKL-40, Eotaxin, and PF4.

FIGS. 27A-27B depict blood biomarker levels versus time. FIG. 27A depicts CRP levels versus time. Blood samples were acquired within about 1 day, 24-36 hours, of hospital admission, and where possible at about 7 days, 14 days and 8 weeks after admission, for COPD exacerbating and recovering patients. FIG. 27B depicts combined blood biomarker levels versus time. Blood samples were acquired within about 1 day, 24-36 hours, of hospital admission, and where possible at about 7 days, 14 days and 8 weeks after admission, for COPD exacerbating and recovering patients. The combination of biomarkers shown are YKL-40, fibronectin, SAA1, eotaxin1 and sST2 (or IL1RL1).

FIG. 28A-28D illustrate marker levels versus forest algorithm predictions. FIG. 28A illustrates marker levels versus forest algorithm predictions for sRAGE. FIG. 28B illustrates marker levels versus forest algorithm predictions for YKL-40. FIG. 28C illustrates marker levels versus forest algorithm predictions for IgE. FIG. 28D illustrates marker levels versus forest algorithm predictions for Cathepsin S.

FIG. 29A-29D illustrate incidence rates for COPD exacerbations as a function of percentiles cut off values for four representative biomarkers. FIG. 29A illustrates incidence rates for COPD exacerbations as a function of percentiles cut off values for sRAGE. FIG. 29B illustrates incidence rates for COPD exacerbations as a function of percentiles cut off values for Pentraxin 3. FIG. 29C illustrates incidence rates for COPD exacerbations as a function of percentiles cut off values for pro-ANP. FIG. 29D illustrates incidence rates for COPD exacerbations as a function of percentiles cut off values for GDF15.

### DETAILED DESCRIPTION OF THE INVENTION

Chronic obstructive pulmonary disease (COPD) is a complex disease, and as such has previously been difficult to characterize clinically with the use of biomarkers. What is described herein are biomarkers associated with COPD as well as methods of detecting biomarkers associated with COPD. These biomarker and biomarker combinations can be used to calculate a disease score. The disease score can then be used to stratify a patient into a specific risk category, which can then inform patient management decisions.

### Certain Terminologies

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for COPD, if, after receiving a therapeutic amount of a therapeutic agent, the subject shows observable and/or measurable reduction or relief of, or absence of one or more symptom of COPD, reduced morbidity and/or mortality, and improvement in quality of life issues.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH2, F(ab′)2, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies).

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

Disclosed herein are methods for monitoring the progression or disease state of a subject in need thereof. In some cases, the subject in need thereof is diagnosed with, is suspected of having, or is at risk of developing chronic obstructive pulmonary disease (COPD). In one aspect, the method includes performing an immunoassay on a biological sample from the subject. The subject can be a human. In another aspect, the method includes performing a plurality of immunoassays on a biological sample from the subject. The biological sample can be any sample obtained from the subject, including, without limitation, blood, serum, plasma, sputum and the like. In some cases, the biological sample is obtained from the subject during a visit to the clinic or the hospital. In some cases, the methods are utilized to predict or monitor the progression of a subject during an acute COPD-related exacerbation event. An acute COPD-related exacerbation event may be a sudden worsening of COPD symptoms (e.g., shortness of breath, quantity and color of phlegm) that may last for a few days. Acute exacerbations may be triggered by a bacterial or viral infection or by environmental pollutants. Airway inflammation may increase during the exacerbation resulting in increased hyperinflation, reduced expiratory air flow and worsening of gas transfer. In some cases, it may be difficult to determine whether the subject undergoing an exacerbation event is likely to progress to a worsening of symptoms or if the subject will stabilize without strong therapeutic intervention. Further, the administration of therapeutics to treat and/or stabilize the exacerbation event may render it difficult to predict the outcome of the subject. For example, the subject may be treated after admission to the hospital and then released after the symptoms of the exacerbation event have subsided, only to relapse with more severe symptoms days later. In some cases, the methods provided herein may involve the measurement of a biomarker signature that may allow a healthcare practitioner to predict the outcome of the subject and to prescribe the proper course of treatment.

In some aspects, the method may involve performing a plurality of immunoassays on a biological sample obtained from the subject and detecting the levels of a plurality of biomarkers present in the sample. In some embodiments, the plurality of biomarkers comprises two or more, three or more, or four or more biomarkers. In some embodiments, the plurality of biomarkers comprises three, four, five, six, seven, eight, nine, ten, or more than ten biomarkers. In some embodiments, the plurality of biomarkers comprises three biomarkers. In some embodiments, the plurality of biomarkers comprises four biomarkers. In some embodiments, the plurality of biomarkers comprises five biomarkers. In some aspects, the method involves performing a plurality of immunoassays on a biological sample obtained from the subject and detecting the levels of the plurality of biomarkers present in the sample. The plurality of immunoassays can be performed in different reactions. In one example, the different reactions can be carried out in different wells of a microplate. The plurality of immunoassays can be performed in the same reaction. In one example, the same reaction can comprise multiple different capture antibodies. Alternatively, the plurality of immunoassays can comprise at least one reaction detecting a single biomarker and at least one reaction detecting two or more biomarkers.

In some cases, the plurality of biomarkers are selected from the following classes of molecules: a platelet degranulation product, a cathepsin, an endopeptidase, an endopeptidase inhibitor, a cystatin, a serpin, an immunoglobulin, a coagulation protein, a fibrosis or fibrinolysis protein, a fibrin degradation product, a protein involved in platelet activity, a chemotaxis protein, a chemokine produced by an immune response, an interleukin receptor or receptor-like protein, a Toll-like receptor or protein with Toll-like receptor domains, a complement pathway protein, a leukocyte related protein, an adipokine, an adipose-derived hormone, a protein involved in the insulin pathway, a protein involved with insulin resistance, a protein involved with calcium homeostasis, an acute phase protein, a pentraxin, a natriuretic peptide, a lipoprotein, an advanced glycation end-product, an extracellular glycoprotein, an apolipoprotein, a chitinase, a protein from the transforming growth factor beta superfamily, and a club cell related protein. In some cases, the biomarkers are selected from Table 1 as described below. In some cases, the four or more biomarkers include four, five, six, seven, eight, nine, ten, or more than ten biomarkers.

In some cases, a first biomarker from said plurality of biomarkers is selected from the group consisting of: an advanced glycation end-product, a platelet degradation product, a coagulation protein, a protein involved in platelet activity, a chemotaxis protein, a chemokine produced by an immune response, an endopeptidase inhibitor, a club cell rated protein, a protein involved with calcium homeostasis, and a natriuretic peptide. For example, the first biomarker can be selected from the group consisting of: soluble Receptor for Advanced Glycation End products, Platelet Factor 4, P-selectin, Regulated on Activation Normal T Cell Expressed and Secreted (RANTES), Tissue Inhibitor of Metalloproteinase 1, Pulmonary and Activation-Regulated Chemokine, Club cell 16 protein, pro-peptide of atrial natriuretic peptide, and Fibrinogen. The first biomarker can also be a pentraxin. In some cases the pentraxin is CRP.

In some cases, a second biomarker from said plurality of biomarkers is selected from the group consisting of: a pentraxin, a complement pathway protein, an adipokine, a protein involved in the metabolic pathway, a coagulation protein, a degradation product of fibrin, an acute phase protein, a chemotaxis protein, a chemokine produced by an immune response, a cathepsin, and a cystatin. For example, the second biomarker can be selected from the group consisting of: C-Reactive Protein, Pentraxin 3, Adiponectin, D-Dimer, Interleukin 6, Monocyte chemoattractant protein-1, Cathepsin S, and Cystatin C. In some cases, the second biomarker is not a pentraxin. In some cases, the second biomarker is not CRP.

In some cases, a third biomarker from said plurality of biomarkers is selected from the group consisting of: an acute phase protein, a leukocyte or neutrophil related protein, a protein involved in platelet activity, an immunoglobulin, a coagulation protein, a serpin, an endopeptidase inhibitor, and a chitinase. For example, the third biomarker can be selected from the group consisting of: Serum amyloid A-1, Human Neutrophil Lipocalin, Growth Differentiation Factor 15, Immunoglobulin A, Fibronectin, Alpha-1 Antitrypsin, Chitinase 3-like 1, and Pro-calcitonin.

In some cases, a fourth biomarker from said plurality of biomarkers is selected from the group consisting of: an adipokine, an adipose derived hormone, a protein involved in the metabolic pathway, a protein involved with insulin resistance, an immunoglobulin, a chemotaxis protein, an eosinophil related protein, a complement pathway protein, a matrix metallopeptidase, an interleukin receptor or receptor-like protein, a toll-like receptor or protein with toll-like receptor domains, and a leukocyte or neutrophil related protein. For example, the fourth biomarker can be selected from the group consisting of: Leptin, Immunoglobulin E, Eotaxin, Complement component 1q, soluble ST2, Matrix Metallopeptidase 9, Neutrophil Elastase, and Resistin.

At least of the plurality of biomarkers can be sRAGE. The at least one of the plurality of biomarkers can be sRAGE if the disease score is tailored to a clinical group that includes structural, functional, or symptomatic aspects of emphysema. At least of the plurality of biomarkers can be Pentraxin 3. The at least one of the plurality of biomarkers can be Pentraxin 3 if the disease score is tailored to a clinical group that includes structural, functional, or symptomatic aspects of chronic bronchitis, bronchiectasis, or early or relatively asymptomatic functional decline. At least of the plurality of biomarkers can be NT-proANP. The at least one of the plurality of biomarkers can be NT-proANP if the disease score is tailored to a clinical group including cardiovascular disease, metabolic dysfunction, or a combination thereof. At least of the plurality of biomarkers can be IgA. The at least one of the plurality of biomarkers can be IgA if the disease score is tailored to a clinical group including patients with aspects of immune deficiency. In some cases, CRP is not included in the plurality of biomarkers. In some cases, fibrinogen is not included in the plurality of biomarkers.

The platelet degranulation product can be RANTES, PF4, or P-selectin. The cathepsin can be Cathepsin C. The cystatin can be Cystatin C. The endopeptidase inhibitor can be a TIMP, A2M, A1At, or a serpin. The TIMP can be TIMP-1, TIMP-2, TIMP-3, or TIMP-4. The serpin can be a protease inhibitor, such as a serine protease inhibitor. The serine protease inhibitor can be trypsin, thrombin, or neutrophil elastase. The immunoglobulin can be IgA, IgE, or IgG. The IgA can be total IgA, IgA1, or IgA2. The IgG can be total IgG, IgG1, IgG2, IgG3, or IgG4. The coagulation protein, fibrinolysis, or fibrin degradation product can be D-Dimer, PF4, fibrinogen, fibronectin, or A2M. The protein involved in platelet activity can be Growth Differentiation Factor 15 (GDF-15), Vascular Endothelial Growth Factor (VEGF), VEGF receptors, PF4, P-selectin, or RANTES. The chemotaxis protein can be Eotaxin-1 (CCL11), RANTES (CCL5), PARC (CCL18), MCP1 (CCL2), or PF4 (CXCL4). The chemokine produced by an immune response can be Monocyte Chemoattractant Protein 1 (MCP-1), PARC, Platelet Factor 4 (PF4), or RANTES. The interleukin receptor or receptor like product can be IL-1β, IL-5, IL-4, IL-6, IL-13, IL-17A, IL-33, or ST2. The Toll-like receptor or protein with Toll-like receptor domains can be ST2 or HMGB1. The complement pathway protein can be C1q, PTX3, or MBL. The eosinophil related protein can be Eotaxin-1, ECP, or eosinophil counts. The leukocyte or neutrophil related protein can be Human Leukocyte elastase or Neutrophil elastase, Human Neutrophil Lipocalin, Resistin, MPO, white blood cell counts, or neutrophil counts. The adipokine can be adiponectin or leptin. The adipose-derived hormone can be leptin or resistin. The protein involved in a metabolic pathway can be leptin, adiponectin, resistin, insulin, or A1c. The protein involved with calcium homeostasis can be NT-proANP. The acute phase protein can be SAA-1, IL6, TNFa, CRP, PTX3, or pro-calcitonin. The pentraxtin can be CRP or PTX3. The natriuretic peptide can be NT-ProANP. The lipoprotein can be SAA-1, low density lipoprotein (LDL), or high density lipoprotein (HDL). The lipoprotein can be an apolipoprotein. The advanced glycation end-product can be sRAGE, HMGB1, calprotectin, or S100A8/A9. The extracellular glycoprotein can be OSF-2 or MBL. The matrix metallopeptidase can be MMP-7, MMP-9, or MMP-12. The chitinase can be YKL-40. The protein from the transforming growth factor beta super family can be TGFβ. The club cell related protein can be CC16.

In some cases, the levels of the plurality of biomarkers are measured by performing a plurality of immunoassays. In some cases, the plurality of immunoassays comprises two or more immunoassays. In some cases, the plurality of immunoassays comprises two immunoassays. In some cases, the plurality of immunoassays comprises three immunoassays. In some instances, the plurality of immunoassays comprises four immunoassays. In some cases, the plurality of immunoassays comprises five, six, seven, eight, nine, ten, or more than ten immunoassays.

In some cases, the plurality of immunoassays are the same immunoassay (e.g., four or more ELISA assays). When the plurality of immunoassays are the same immunoassay, each of the plurality of immunoassays can detect a different biomarker. When the plurality of immunoassays are the same immunoassay, each of the plurality of immunoassays can be performed in the same reaction chamber or a different reaction chamber. A reaction chamber can be any suitable space for performing an immunoassay. Examples of reaction chambers include, but are not limited to, a well in a microplate, an Eppendorf tube, or a droplet.

In some cases, the plurality of immunoassays are different immunoassays (e.g., an ELISA assay and an AMMP® assay). When the plurality of immunoassays are different immunoassays, each of the plurality of immunoassays can detect a different biomarker. When the plurality of immunoassays are different immunoassays, each of the plurality of immunoassays can be performed in the same reaction chamber or a different reaction chamber.

In some cases, the measurement of the four or more biomarkers may be affected or hindered by the use of an immunoassay with an optical readout. In some cases, at least one of the plurality of immunoassays is a non-optical assay. In some cases, at least two of the plurality of immunoassays are non-optical assays. In some aspects, at least three of the plurality of immunoassays are non-optical assays. In some instances, all of the plurality of immunoassays are non-optical assays. In some instances, the non-optical immunoassay is an acoustic immunoassay. In some aspects, the acoustic immunoassay is an acoustic membrane microparticle (AMMP®) assay. In some cases, the non-optical assay is more sensitive to low concentrations of a biomarker than an optical assay (e.g. ELISA). The non-optical assay can be 3× to 10× more sensitive to a low concentration of a biomarker than the optical assay. The non-optical assay can be 3×, 4×, 5×, 6×, 7×, 8×, 9×, or 10× more sensitive to a low concentration of a biomarker than the optical assay. In some cases, the non-optical assay enables performing an assay on a biological sample with low or no dilution of the biological sample. In some aspects, the non-optical assay enables detection of protein interactions or complexes. In some instances, the biological sample is blood, serum, sputum, plasma, tissue lysate, or urine. Non-limiting examples of other immunoassays amenable for use with the methods described herein include enzyme-linked immunosorbent assays (ELISA), homogeneous immunoassays, Western blots, fluorescence immunoassays, chemiluminescence immunoassays, electro-chemiluminescence immunoassays, fluorescence resonance energy transfer (FRET) immunoassays, time resolved fluorescence and/or FRET immunoassays, lateral flow immunoassays, microspot (fluorescence) immunoassays, surface plasmon resonance immunoassays or ligand assays, clotting assays, immune-capture coupled with mass spectrometry, and the like. In some cases, the immunoassays are single-plexed. In some cases, the immunoassays are multiplexed.

In some aspects, the method comprises calculating a disease score. The disease score can represent a disease activity of COPD. The disease score may be a numerical value, such as a composite score, that relates the levels of the plurality of biomarkers to a disease state. For example, a disease score may indicate that a subject is likely to relapse from an acute exacerbation event. In other examples, a disease score may indicate that a subject is likely to recover from an acute exacerbation event. In some examples, the disease score may be correlated with a particular course of treatment, for example, over long or short terms. In some cases, the disease score is compared with a predetermined cutoff or reference value associated with an increased risk of unstable or acute COPD events. In some cases, the methods further include presenting the disease score on a report.

The disease score can be selected from a numerical value of said disease activity, a categorization of the disease activity above a cutoff, a categorization of the disease activity below a cutoff, a classification of the disease activity into a category, and a combination thereof. The diseases score can provide a measure of disease activity. The disease score can represent stratification of increasing disease activity. The disease activity can be a measure of exacerbations, a measure of exacerbation frequency, a measure of exacerbation severity, a measure of a risk of future exacerbation activity, a measure of lung function, COPD related symptoms, a vital sign, a measure of exercise tolerance, a measure of exertion tolerance, a measure of frailty, or a combination thereof. Examples of COPD related symptoms include, but are not limited to dyspnea and the ability to function with exertion. Examples of vitals include, but are not limited to, circulatory measures and oxygen saturation. Exercise tolerance can be determined from a walk test (i.e. a 6 minute walk test, or a walk test for an adjusted amount of time), a distance walked in a specified amount of time, stair climbing, repetitive sitting and standing from a chair, or a combination thereof. The measure of frailty can be determined from a questionnaire answered by the subject.

In some cases, the disease score is a numerical value, for example a value from 0 to 100 or from 1 to 100. The cutoff can be a value of a disease score pre-determined to be clinically relevant. The category can be a category of patient population of interest, such as, for example, a population with controlled chronic obstructive pulmonary disease, a population with uncontrolled chronic obstructive pulmonary disease, a population prone to a future acute exacerbation event, a population not prone to a future acute exacerbation event, a population which will benefit from an increased therapy, a population which will benefit from a decreased therapy, and a combination thereof. Classification of disease activity into a category or plurality of categories, i.e. patient stratification, can provide health management options for the subject to a healthcare provider or to the subject.

In some cases, calculating a disease score comprises normalization of at least one biomarker. In some cases, normalization of at least one biomarker comprises the at least one biomarker level incorporated in at least one calculation term with a negative exponent of the biomarker level or negative coefficient multiplying a logarithm transformation of the biomarker level. In some cases, normalization of at least one biomarker comprises logarithm transformation of the level of the at least one biomarker. In some cases, the calculating involves multiplication, or addition of logarithm transformation, of the levels of the plurality of biomarkers. In some cases, differing analytical combinations of biomarker levels are assessed by logical relationships for associated sub— groups of COPD subjects, where the logical relationships may include risk factors, such as smoking status, gender, and/or age, or clinical parameters such as body mass, blood pressure, or temperature, and additionally or alternatively may include select molecular biomarker levels or combinations of two or more biomarkers levels.

In some cases, the methods involve performing an immunoassay to measure a level of a molecular complex present in a biological sample. A molecular complex may include two or more molecules (e.g., proteins) in association or bound to one another. A molecular complex may include two molecules bound or associated or may include higher order complexes, for example, more than two molecules bound or associated. In some cases, the presence of a molecular complex in a biological sample may indicate a disease state of the subject. In one example, the methods provided herein include measuring the levels of PF4-RANTES complexes in the biological sample. In some cases, the levels of PF4-RANTES complex may be an indicator of disease state of a COPD patient. For example, an increased level of PF4-RANTES complex may indicate that the COPD patient has an increased risk of an eminent or a recurring exacerbation event. In some cases, the levels of an alpha-1 antitrypsin (A1AT) may be measured. In this example, the levels of PF4-RANTES complex may be normalized to the levels of A1AT (i.e., a ratio of PF4-RANTES/A1AT). In some cases, the methods involve independently measuring the levels of PF4 and RANTES and multiplying them together to give a measure (“PF4×RANTES”). In some cases, the methods involve normalizing the PF4×RANTES measure with A1AT levels (e.g., (PF4×RANTES)/A1AT)) to give an indication of eminent exacerbation.

In some cases the plurality of biomarkers are selected from Table 1 of the specification. In some cases the plurality of biomarkers are selected from those indicated in examples 1 through 9 in the specification. In some cases the plurality of biomarkers are selected from the group consisting of: Alpha-1 antitrypsin (A1AT), a-2-Macroglobulin (A2M), Adiponectin, C1q, Calprotectin, Cathepsin S, Club cell 16 protein (CC16), C-reactive protein (CRP), Cystatin C, D-dimer, Eotaxin-1 (CCL11), Eosinophil Cationic Protein (ECP), Fibrinogen, Fibronectin, Growth Differentiation Factor 15 (GDF-15), Human Neutrophil Lipocalin (HNL), High Mobility Group 1 (HMGB1), IgA, IgE, IgG, IL-5, IL-4, IL-6, IL-13, IL-17A, IL-33, Leptin, Mannose-Binding Lectin (MBL), Monocyte Chemoattractant Protein 1 (MCP-1), Matrix metallopeptidase 7 (MMP-7), Matrix metallopeptidase 8 (MMP-8), Matrix metallopeptidase 9 (MMP-9), Matrix metallopeptidase 12 (MMP-12), Myeloperoxidase (MPO), Neutrophil Elastase, PARC, Pro-calcitonin (PCT), Pentraxin 3 (PTX3), Periostin (OSF-2), Platelet Factor 4 (PF4), NT-ProANP, P-Selectin, RANTES, Resistin, Serum amyloid A-1 (SAA-1), soluble Receptor for Advanced Glycation End products (sRAGE), soluble ST2, Tissue Inhibitor of Metalloproteinases 1 (Timp-1), TNF-α, Vascular Endothelial Growth Factor (VEGF), and Chitinase 3-like 1 (CHI3L1, YKL-40).

In some cases the plurality of biomarkers comprises at least two of RANTES, PF4, P-selectin, A1AT, Neutrophil Elastase, Cathepsin S and Cathepsin G. In some cases the plurality of biomarkers comprises at least two of CRP, MMP-9, TIMP1, IgA, SAA1, PF4 and RANTES. In some cases the plurality of biomarkers comprises at least two of IgA, Adiponectin, PF4 and RANTES. In some cases the plurality of biomarkers comprises at least two of PF4, P-selectin, MMP-9, TIMP1, C1q, and C3a. In some cases the plurality of biomarkers comprises at least two of PF4, P-selectin, MMP-9, TIMP1, C1q, Adiponectin. In some cases the plurality of biomarkers comprises at least two of sRAGE, TIMP-1, Leptin, Adiponectin, Fibronectin, YKL-40, IgE, Eotaxin1, P-Selectin, PF4, MCP-1, CRP, SAA1, PCT, MMP-9, IgA, C1q, and HNL. In some cases the plurality of biomarkers comprises at least two of Fibrinogen, CRP, HNL, fibronectin, MMP-9, IgA, MCP-1, sRAGE, PCT, IgE, Adiponectin, P-selectin, Leptin, SAA1, TIMP-1, C1q, resistin, HbA1c and insulin. In some cases the plurality of biomarkers comprises at least two of sRAGE, Eotaxin, HNL, IL6, PF4, P-selectin, YKL-40, SAA1, and RANTES. In some cases the plurality of biomarkers comprises at least two of HNL, PF4, P-selectin, sRAGE, CRP, MMP-9, IgA, Eotaxin, C1q and MCP-1. In some cases the plurality of biomarkers comprises at least two of RANTES, PF4, P-selectin, Fibrinogen, YKL-40, PCT, SAA1, Eotaxin1, PARC, Leptin, IgA, MMP-9, C1q, and CRP. In some cases the plurality of biomarkers comprises at least two of RANTES, PF4, P-selectin, Leptin, MCP-1, Adiponectin, IgA, Eotaxin1, IgE, sRAGE, Fibrinogen, SAA1, CRP, Fibronectin, C1q, and sST2 (IL1RL1). In some cases the plurality of biomarkers comprises at least two of YKL-40, sRAGE, PCT, MCP-1, IL6, MMP-9, Fibronectin, Eotaxin, P-selectin, Leptin, IgA, SAA1, CRP and sST2 (IL1RL1). In some cases the plurality of biomarkers includes at least one of CRP, fibrinogen, ANP, NT-proANP, BNP, NT-proBNP, D-Dimer, and sST2. In some cases the plurality of biomarkers includes at least one of resistin, insulin, blood glucose and hA1c. In some cases the plurality of biomarkers comprises at least two of fibronectin, SAA1, Eotaxin1, sST2 (IL1RL1), cardiac troponin, MCP-1, YKL-40, IL6, IgE and IgA. In some cases the plurality of biomarkers comprises at least one of C3 (total), C3a, C3c, C3d, iC3b, C5a, SC5b-9, and C4a. In some cases the plurality of biomarkers comprises at least one of the adipokines related to C1q and TNF like, CTRP-1, CTRP-3, CTRP-5, CTRP-9, and CTRP-15.

In some cases, the plurality of biomarker selections are rationalized from the identified biochemical pathways activated and are grouped and associated with respect to clinical measures provided in the examples of this specification. In some cases, the four or more biomarkers comprise at least four of HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, IgA, Neutrophil Elastase, Leukocyte Elastase, Cathepsin S, Cathepsin G, Thrombopoietin, Haptoglobin, Pentraxin 3, IL1beta, IL4, MMP-9, TIMP1, C1q, PARC, BNP, ANP, NT-proANP, cTnI, Cystatin C, D-Dimer, Resistin, Insulin, GDF15 and CC16.

In some cases, the four or more biomarkers comprise at least four of sRAGE, PARC, Leptin, RANTES, IgA, C1q, IL-6. In some cases, said four or more biomarkers comprise at least four of sRAGE, IL-6, Leptin, HNL, Adiponectin and a quantitative CT measure of small airway disease. In some cases, said four or more biomarkers comprise at least four of HNL, Leptin, IgE, YKL-40, P-selectin, IgA, TIMP-1, SAA1, IL-6, and age. In some cases, the four or more biomarkers comprise at least HNL, IgE, Leptin and age. In some cases, the four or more biomarkers comprise at least four of HNL, PCT, PF4, P-selectin, IgE, IL-6, Eotaxin1, SAA1, PARC, TIMP-1, IgA, sRAGE. In some cases, the four or more biomarkers comprise at least four of IgE, PCT, PF4, P-selectin, HNL, Eotaxin1, PARC, IL-6. In some cases, the four or more biomarkers comprise at least four of HNL, PF4, P-selectin, PCT, IgE, SAA1, sRAGE, PARC, IL-6, IgA, IgA, TIMP-1 and a symptoms score (e.g. COPD Assessment Test—CAT—score, Saint Georges Respiratory Questionnaire—SGRQ —symptoms score). In some cases, the four or more biomarkers comprise at least four of IL-6, MMP-9, IgA, PCT, IgE, HNL, PARC and gender. In some cases, the four or more biomarkers comprise at least four of MMP-9, SAA1, PF4, P-selectin, HNL, sRAGE, TIMP-1, CRP, YKL-40 and gender.

In some cases, the four or more biomarkers comprise at least four of SAA1, Eotaxin1, C1q, NT-ProANP, IL-6, GDF-15, IgE, IgA, sRAGE. In some cases, the four or more biomarkers comprise at least four of Leptin, GDF-15, IgE, TIMP-1, MMP-9, Eotaxin, NT-proANP and gender. In some cases, the four or more biomarkers comprise at least four of SAA1, Adiponectin, C1q, IL-6, Eotaxin and gender. In some cases, the four or more biomarkers comprise at least four of SAA1, IgE, Eotaxin, NT-proANP, GDF-15. IL-6, IgA, C1q, TIMP-1, Adiponectin and a symptoms score (e.g. CAT or SGRQ score). In some cases, the four or more biomarkers comprise at least four of GDF-15, IgE, Leptin, MMP-9, NT-proANP, TIMP-1, a symptoms score (eg. CAT or SGRQ) and gender. In some cases, the four or more biomarkers comprise at least four of Adiponectin, SAA1, NT-proANP, P-selectin, IL-6, Eotaxin, symptoms score (eg. CAT) and gender.

In some cases, the four or more biomarkers comprise at least four of sRAGE, Eotaxin1, C1q, HNL, IgE, A1AT, TIMP-1, MMP-9, D-Dimer. In some cases, the four or more biomarkers comprise at least one of gender, history of 2 or more treated exacerbations in past 12 months, symptoms score (CAT or SGRQ scores), inhaled steroids use, forced expiratory volume in 1 second (FEV1), smoking and age. In some cases, the four or more biomarkers comprise at least four of C1q, HNL, Eotaxin1, sRAGE, Cathepsin S, Resistin, IgE, YKL-40, PF4, Neutrophil Elastase, A1AT, P-selectin, MCP-1, and symptoms score (e.g. CAT or SGRQ scores). In some cases, the four or more biomarkers comprise at least four of C1q, sRAGE, Eotaxin1, Resistin, HNL, A1AT, YKL-40, IgE, Cathepsin S, Neutrophil Elastase, RANTES, PF4, P-selectin and symptoms score (e.g. CAT or SGRQ scores).

In some cases, the plurality of biomarkers comprise at least four of HNL, CRP, sRAGE, SAA1, Fibrinogen, Leptin, Adiponectin, IgE, Eotaxin1, YKL-40, MCP-1, IL6, PCT, Fibronectin, RANTES, PF4, P-selectin, A1AT, sST2, NT-proBNP, and IgA. In some cases, the plurality of biomarkers comprise of at least three of HNL, CRP, sRAGE, SAA1, Fibrinogen, Leptin, Eotaxin1, YKL-40, PCT, RANTES, PF4, P-selectin, A1AT, sST2, and NT-proBNP. In some cases, the plurality of biomarkers comprise of at least two of sRAGE, SAA1, Leptin, Eotaxin1, YKL-40, PCT, sST2, and NT-proBNP. In some cases, the plurality of biomarkers includes at least two of YKL-40, sRAGE, PCT, MCP-1, IL6 and sST2. In some cases, the plurality of biomarkers includes at least two of Leptin, Eotaxin1, Adiponectin, MCP-1, SAA1 and IgE. In some cases, the plurality of biomarkers includes two of PF4, P-selectin, RANTES, Fibrinogen and Fibronectin. In some cases, the plurality of biomarkers includes at least two of sST2, CRP, Eotaxin1, Fibronectin, MCP-1 and SAA1. In some cases, the plurality of biomarkers includes at least two of HNL, sRAGE, MMP-9, TIMP1, CRP and IgA. In some cases, the plurality of biomarkers includes sST2.

The some cases the plurality of biomarkers comprises at least one biomarker selected from sRAGE, PF4, P-selectin, RANTES, TIMP1, PARC, CC16, NT-proANP, and Fibrinogen. In some cases, the plurality of biomarkers comprises at least one biomarker selected from CRP, Pentraxin 3, sST2, D-DIMER, IL6, MCP-1, Cathepsin S, and Cystatin C. In some cases, the plurality of biomarkers comprises at least one biomarker selected from SAA, HNL, GDF 15, IgA, Fibronectin, NT-proANP, A1AT, YKL-40, and PCT. In some cases, the plurality of biomarkers comprises at least one biomarker selected from Leptin, IgE, Eotaxin, C1q, adiponectin, MMP-9, Neutrophil Elastase, and Resistin. In some cases, at least one biomarker of the plurality of biomarkers has a non-monotonic contribution to the disease score, wherein the at least one biomarker is selected from sRAGE, Leptin, adiponectin, PTX3, YKL40, GDF 15, PARC, Fibronectin, IgE, Eotaxin, Cystatin C, NT-proANP, TIMP1, D-Dimer. In some cases, at least one biomarker of the plurality of biomarkers is indicative of a contribution from at least one protein complex, wherein the at least one biomarker is selected from A1AT, IgA, C1q, CRP, PTX3, sRAGE, HMGB1, calprotectin, PF4, RANTES, Cystatin C, MMP-9, TIMP-1, YKL-40.

In some cases, the plurality of biomarkers together and/or independently have association during, after and prior to acute exacerbations of COPD. In some cases, the plurality of biomarkers are associated with early stage of disease, mid stage of disease, late stages of disease, or a combination thereof. A disease activity algorithm for a patient suffering from COPD, or similar small airways related disease(s), can be formulated from at least four biomarkers described herein. The disease activity algorithm can be used to generate a disease score. The disease score can further comprise an additional clinical parameter, further described herein. The disease score can indicate whether a patient's COPD is controlled or uncontrolled, whether the patient may be prone to near or further term acute events, or whether the patient may benefit from, or has benefitted from increased or decreased therapy and pharmacological treatment of the disease and disease aspects.

In some aspects, the disease score may be further supplemented with one or more additional parameters. The one or more additional parameters may serve to fine-tune or further differentiate the biomarker signatures. In some cases, the one or more additional parameters include one or more clinical parameters. The one or more clinical parameters may include an age, a race, a sex, a gender, a blood pressure measurement, a temperature, a weight, a height, a body mass index, an anthropometric measurement, strength, exercise tolerance, an estimated blood volume or a combination thereof of the subject. In some cases, the one or more clinical parameters may include a disease classification by Global Initiative for Chronic Obstructive Lung Disease (GOLD) guidelines, one or more spirometry parameters, symptoms assessed by COPD Assessment Test score, symptoms assessed by modified Medical Research Council score, COPD exacerbations counted as presentation of acute worsening of respiratory symptoms that is treated, COPD exacerbations counted as presentation of acute worsening of respiratory symptoms by physician's classification, symptoms assessed by modified Borg Scale, symptoms assessed by Baseline or Transition Dyspnea Indices, symptoms assessed by UCSD shortness of breath questionnaire, symptoms assessed by American Heart Association Dyspnea Index, symptoms assessed by Saint Georges Respiratory Questionnaire or any combination thereof.

In some cases, the one or more additional parameters may include one or more imaging parameters. The one or more imaging parameters may include, for example, a Computed Tomography (CT) image. The CT image can be a quantitative CT (QCT). The CT image may include low attenuation area at max inspiration, low attenuation area at max expiration, airway wall area or airway wall thickness, a measure of gas trapping or hyperinflatation, or parametric measures of emphysema or small airway disease, or any combination thereof.

In some cases, the one or more additional parameters may include one or more variables representative of pulmonary function. For example, the one or more variables representative of pulmonary function can be FEV1/FVC, FEV1 in liters, FVC in liters, FEV1 in percent predicted value, FEV1 reversibility, residual volume/total lung capacity ratio, or a combination thereof. FEV1, or forced expiratory volume in 1 second can be the maximum amount of air a subject can forcefully blow out of their lungs in one second. FVC, or forced vital capacity, can be the amount of air which a subject can forcibly exhale from their lungs after taking the deepest breath possible.

In some cases, the one or more additional parameters may include one or more scores representative of a symptom of the individual. The one or more scores representative of a symptom can be a score of dyspnea, dyspnea on exertion, dyspnea on performing daily activities, cough, phlegm production, chest tightness, sleep quality, energy level, and confidence levels.

In some cases, the one or more additional parameters may include one or more variables representative of the individual's exacerbation history. The exacerbation history can be examined in a time frame ranging from the past 1 month to past 24 months. The exacerbation history can be examined in a time frame, selected from the group consisting of the past 1 month, 2 months, 3 months, 4 months, 5 months, 6 months, 7 months, 8 months, 9 months, 10 months, 11 months, 12 months, 15 months, 18 months, and 24 months. The one or more variables representative of the individual's exacerbation history can be occurrence of a symptom in the frame, the number of times the symptom occurred in the time frame, urgency of the symptom, or a combination thereof. The urgency of the symptom can be determined in the form of setting of care received, for example out-patient call in, video call, clinic visit, emergency department use, hospital admission, or hospital admission with intubation.

In some cases, the one or more additional parameters may include one or more variables representative of current medication use of the individual. The current medication use of the individual can include use by the individual of a steroid, a long-acting beta2 agonist (LABA), a long-acting muscarinic antagonist (LAMA), a phosphodiesterase (PDE) inhibitor, an anti-inflammatory, an antibiotic, a biologic, a complement pathway inhibitor, a supplement or augmentation for deficiencies, or a combination thereof. Antibiotic use can comprise chronic use of low dose macrolides. Biologic use can comprise use of biologics targeted to interfere with immunological pathways.

In some cases, the one or more additional parameters may include one or more variables representative of a comorbid condition of the individual. A comorbid condition can be a metabolic disorder, a vascular disorder, a circulatory disorder, a cardiac disorder, an additional lung disorder, a liver disorder, a gastrointestinal disorder, a CNS disorder, or a combination thereof.

In some cases, the one or more additional parameters may comprise one or more variables representative of time of year or season. The time of year or season can be a time of year or season an exacerbation event occurred, time of year or season a treatment was administered, or a time of year or season a sample was taken from the patient. A variable representative of time of year can be a month. A variable representative of time of year or season can be a numerical value (for example, to represent time of year, a single numeric value could be assigned from 1 through 365 depending a specific day of the year, or 1 through 12 depending on the month; alternatively, to represent season a value of 1 through 4 could be assigned, each value representing one of the four seasons).

In some cases, the one or more additional parameters may be used to place patients into groups for which combination biomarker signatures or score ranges may have a different relevance. In one non-limiting example, COPD status (as assessed by GOLD) as well as the smoking status of a subject could be used to group patients (e.g., “smoking” versus “active smoker” versus “inactive smoker”). In another non-limiting example, data about inhaled steroid use could be used to group patients (e.g., “daily use” versus “occasional use” versus “never used”). In another non-limiting example, patients can be grouped by gender and age information. In yet another non-limiting examples, patients can be grouped by blood pressure (e.g., systolic blood pressure <110, between 110 and 130, and >130). In another non-limiting example, patients could be grouped by statin use.

The biomarker signatures as described herein may further be supplemented with risk factor data, for example, the smoking status or smoking history of the subject, activity level or inactivity level of the subject, body mass, body mass index, or a combination thereof. In addition, or alternatively, the subject may suffer from additional diseases or disorders consequential to or independent of COPD and may be undergoing treatment for these additional diseases or disorders. These additional factors may complicate the prognosis of the subject and may complicate the underlying molecular signature. These additional risk factors may be accounted for in the methods provided herein. For example, the subject may suffer from hypertension and may receive blood pressure lowering medications. In another example, the subject may suffer from cardiovascular disease and may receive statin and ACE medications. In another example, the subject may suffer from diabetes and may receive TZD and metformin medications. In another example, the subject may suffer from GERD and may receive a proton pump inhibitor.

In some aspects, the disease score is presented on a report. The report may be printed on a tangible medium (e.g., paper) or may be presented on a display (e.g., computer monitor). The report may be relayed to a healthcare practitioner or to the subject directly. In some cases, the healthcare practitioner may prescribe or administer a treatment to the subject based on the disease score. For example, the disease score may indicate that the subject is worsening requiring more aggressive treatment, the subject is relapsing and requires further treatment, the subject is recovering and treatment should be tapered or halted, or the subject is not responding to a current therapy and treatment should be adjusted or altered. In some cases, the treatment may be selected from Table 1 as described below.

In some aspects, the methods described herein may be performed at a given time point to assess a disease status of the subject at that particular time point. In other aspects, the methods are performed more than once to assess the change or progression of COPD in the subject plurality of time points. In some cases, the methods include timing of collection of patient samples with respect to an event or administration of a therapy. In some cases, the event is discharge from a hospital or emergency department. In some cases, a biological sample is obtained 1-90 days after the event. In some cases, a biological sample is obtained 3-30 days after the event. In some cases, a biological sample is obtained 5-21 days after the event.

In one aspect, the methods involve performing an immunoassay on at least a first biological sample taken from the subject at a first time point, wherein the immunoassay detects a level of a plurality of biomarkers. The plurality of biomarkers may be as described herein. The method may further include repeating the immunoassay on at least a second biological sample taken from the subject at a second time point.

In one aspect, the methods involve performing a plurality of immunoassays on at least a first biological sample taken from the subject at a first time point, wherein the plurality of immunoassays detect a level of a plurality of biomarkers. The method may further include repeating the plurality of immunoassays on at least a second biological sample taken from the subject at a second time point.

The method may further include calculating a first and second disease score, wherein calculating the first disease score comprises combining the level of the plurality of biomarkers at the first time point, and wherein calculating the second disease score comprises combining the level of the plurality of biomarkers at the second time point, wherein the first and second disease scores are indicative of a disease status of the subject. The methods may further include identifying a trend of the first and second disease scores from the first time point to the second time point, wherein if the trend of the first and second disease scores are identified as increasing, the subject is identified as relapsing or recurring, and if the trend of the first and second disease scores are identified as decreasing, the subject is identified as recovering. The method may further include presenting the trend on a report.

In some instances, the therapy is a COPD therapy. In some cases, the COPD therapy is selected from the group consisting of: an antibiotic, a steroid, a dilator, an anti-coagulant, a blood thinner, a transfusion of whole or processed blood components, a bronchodilator, a muscarinic antagonist, an anti-inflammatory, a targeted anti-inflammatory, mechanically assisted ventilation, oxygen assistance and any combination thereof.

In some cases, the COPD therapy is a wholistic disease management approach. The wholistic disease management approach can comprise use of a device for engagement with patient. The device can record and transmit a signal. The signal can comprise a measurement of a symptom of the subject, a vital sign of the subject, or a combination thereof. The device can be a wearable device. Examples of vital signs include, but are not limited to, peak expiratory flow (PEF), oxygen (O₂) saturation, heart rate, and body temperature. The device can record and transmit a processed digital image. The device can record and transmit an algorithm synthesized signal that combines clinical factors, vitals, and symptoms entered and measured. The synthesized signal can also include periodic inputs from biomarker algorithms when re-baselining and/or stratifying a patient for a care level. In some cases, the signal is transmitted from a device of the subject to a healthcare provider. The healthcare provider can adjust the COPD therapy based on the signal received from the device of the subject. For example, the healthcare provider can adjust the COPD therapy increasing maintenance treatments or ordering an additional work up, such as a high resolution, time resolved, or contrasted Computed Tomography scan.

In some cases, the disease score stratifies a subject into a risk population. The risk population can be a population in need of disease management or a population not in need of disease management. The population in need of disease management can be a population not currently under disease management. In some cases, the disease score provides a measure of exacerbation risk. In some cases, the exacerbation risk is a chance of recurrence of an acute exacerbation event. Identification of an individual as being in a population in need of disease management can help guide a healthcare provider in choosing appropriate workups and/or therapy for administration to the individual. The disease score can identify a subject as being part of a population, wherein the population is selected from a group consisting of: a population with controlled chronic obstructive pulmonary disease, a population with uncontrolled chronic obstructive pulmonary disease, a population prone to a future acute exacerbation event, a population not prone to a future acute exacerbation event, a population which will benefit from an increased therapy, a population which will benefit from a decreased therapy, or a combination thereof.

In some cases, the method comprises detecting a level of the plurality of biomarkers in a subject at a plurality of time points. In some instances, the plurality of time points comprises two, three, four, five, six, seven, eight, nine, ten, or more than ten time points. In some instances, at least one time point of a plurality of time points comprises a time point before said subject has been treated with the COPD therapy. In some instances, at least one time point of the plurality of time points comprises a time point after said subject has been treated with the COPD therapy (e.g., after admission to the hospital). In some instances, at least one time point of the plurality of time points corresponds to the day the subject is removed from the COPD therapy. In some cases, at least one time point of the plurality of time points is one, two, three, four, five, six, seven, eight, nine, ten or more than ten days after the subject is removed from the COPD therapy (e.g., to allow the subject to biochemically stabilize). In some cases, at least one time point of the plurality of time points is about 5 days after the subject has been treated with the COPD therapy. In some cases, the at least one time point is about 5 days after the subject has been treated with COPD therapy. In some instances, at least one time point of the plurality of time points comprises a time point 3-90 days after said subject has been treated with said COPD therapy. In some instances, at least one time point of the plurality of time points comprises a time point 3-14 days after said subject has been treated with said COPD therapy. In some instances, at least one time point of the plurality of time points comprises a time point 14-30 days after said subject has been treated with said COPD therapy. In some instances, at least one time point of the plurality of time points comprises a time point 14-36 days after said subject has been treated with said COPD therapy. In some instances, at least one time point of the plurality of time points comprises a time point 36-90 days after said subject has been treated with said COPD therapy.

TABLE 1
Classes of disease, associated biomarkers and associated classes
of therapies for use with the methods described herein.

| Mechanisms, Classes, | | |
|---|---|---|
| Paths | Biomarkers | Class of Therapies |
| Platelets, Platelet | Platelet Count | Platelet enrichment |
| Activation, Hyper- | Mean platelet volume | therapy |
| coagulation states | Thrombopoietin | Anticoagulants |
| | Platelet Factor 4 (PF4) | (heparin and |
| | Fibrinogen | heparinoids) |
| | Fibronectin | Targeted inhibitors |
| | D-Dimer | Direct factor Xa |
| | Aα-Val(360) | Thrombin inhibitors |
| | P-selectin | Antithrombin protein |
| | (Pro)thrombin | Non-steroidal anti- |
| | Antithrombin (AT) | inflammatory drugs |
| | Thrombin-Antithrombin III | (NSAIDS) |
| | complex (TAT) | Coxibs |
| | Beta-thromboglobulin (beta- | Batroxobin |
| | TG) | Hementin |
| | Tissue plasminogen activator | |
| | (tPA)/plasminogen activator | |
| | inhibitor (PAI) complex | |
| | von Willebrand factor (VWF) | |
| | Adenosine diphosphate (ADP) | |
| | Thromboxane A2 (TXA2) | |
| | Histamine | |
| | CCL5 (RANTES) | |
| | Interleukin-8 (IL8) | |
| | Interleukin-1-beta (IL1-beta) | |
| | CD40L | |
| | Tissue Growth Factor beta | |
| | (TGFbeta) | |
| | Platelet-derived growth fator | |
| | (PDGF) | |
| | CCL3 | |
| | CCL7 | |
| | CXCL1 | |
| | CXCL5 | |
| | CXCL7 | |
| | Toll-like Receptors (2, 4) | |
| | CCL5-CXCL4 heteromers | |
| | CCL5-CCL17 heteromers | |
| | CXCL4 multimers | |
| Lung Epithelial, | CCL1, CCL2, CCL3, CCL4, CCL5 | |
| Endothelial, | oligomers | |
| Alveolar Insult | Mucins | |
| Response | MUC1 | |
| | MUC5AC | |
| | Calcium-activated chloride | |
| | channel regulator 1 (CLCA1) | |
| | Cystic fibrosis | |
| | transmembrane conductance | |
| | regulator (CFTR) | |
| | Granulocyte-macrophage | |
| | colony-stimulating factor | |
| | (GM-CSF) | |
| | Vascular endothelial growth | |
| | factor (VEGF) | |
| | Epidermal growth factor | |
| | (EGF) | |
| | Surfactant protein D (SP-D) | |
| | Frizzled 8 (FZD8) | |
| | Interleukin-1β (IL1β) | |
| Senescence, Wnt | Prostaglandins | FZD/WNT inhibitors |
| pathway | Prostaglandin E2 (PGE2) | |
| | WNT2 | |
| | WNT2b | |
| | WNT5a | |
| | Secreted frizzled-related | |
| | protein 1 (SFRP1) | |
| | beta-Catenin | |
| Endopeptidases, | MMP -1, -3, -7, -8, -9, -12 | Avasimibe |
| matrix | P-glycoprotein (PGP) | Fluvastatin |
| metalloproteinase | N-alpha-P-glycoprotein (PGP) | Tissue Inhibitors of |
| (MMPs), degraders | Neoepitopes of collagen (e.g., | Metalloproteinases |
| of extracellular | types III, IV, VI) breakdown | (TIMPs) |
| matrix, THP-1 | | Peroxisome-proliferator |
| macrophages | | activated receptor |
| | | (PPARc) agonists |
| | | Troglitazone |
| | | Rosiglitazone |
| | | Pioglitazone |
| | | GW1929 |
| | | PPARa agonists |
| | | Clofibrate |
| | | Fenofibrate |
| | | Pirfenidone |
| Insulin Related | IGFBP1, -2 | Insulin reducing drugs |
| Pathways | Resistin | Thiazolidinediones |
| | Insulin | (TZDs) |
| | Hemoglobin A1c | |
| Neutrophils | Neutrophil Counts | Acebilustat |
| | Neutrophil Elastase | CXCR inhibitors |
| | Myeloperoxidase (MPO) | Alpha-1 Antitrypsin |
| | Resistin | (A1AT) augmentation |
| | SERPINA1 | Inhaled elastase |
| | Cathepsin G | inhibitors |
| | Cathespin S | Elafin |
| | Cystatin C | Carbohydrate-based |
| | Proteinase 3 | inhibitors |
| | Interleukin 8 (IL8) | sLex antagonists |
| | CXCL1 | Bimosiamose |
| | Elafin | Heparins and |
| | Toll-like receptors (TLRs) | heparinoids |
| | | PGX-100 |
| | | PGX-200 |
| | | mAb inhibitors |
| | | EL246 |
| | | Oral p38 MAPK |
| | | inhibitors |
| | | SB 203580 |
| | | SB 239063 |
| | | Doramapimod (BIRB |
| | | 796) |
| | | SD282 |
| | | VX745 |
| | | SCIO469 |
| | | SD0006 |
| | | Dilmapimod |
| | | Losmapimod |
| | | CP690550 |
| | | PH797804 |
| | | BMS582949 |
| | | R1503 |
| | | AW814141 |
| | | Inhaled p38 MAPK |
| | | inhibitors |
| | | ARRY371797 |
| | | PF03715455 |
| | | p38 MAPK antisense |
| | | oligonucleotides |
| | | SCIO469 |
| | | SCIO323 |
| Eosinophils | Eosinophil count | Anti-IL5 mAb |
| | Eosinophil cationic protein | |
| | (ECP) | |
| | Eotaxin-1 | |
| | Interleukin-5 (IL-5) | |
| | Interleukin-3 (IL-3) | |
| | Interleukin-33 (IL-33)/ST2 | |
| | complex | |
| | NADPH complexes | |
| | Toll-like receptors (TLRs) | |
| Leukocytes | Leukocyte count | Leukotriene B4 (LTB4) |
| | Leukotriene B4 (LTB4) | BLT1 antagonists |
| | Lipoxin A4 (LXA4) | LY 29311 |
| | Toll-like receptors (TLRs) | SB 225002 |
| | | SC 53228 |
| | | CP 105696 |
| | | Amelubant (BIIL284) |
| | | LY 29311 |
| | | LTB019 |
| | | SB 201146 |
| | | Dual BLT1 and BLT2 |
| | | antagonists |
| | | RO5101576 |
| | | 5-LO inhibitors |
| | | Zileuton |
| | | MK-0633 |
| | | FLAP antagonist |
| | | BAYx1005 |
| | | Chemokine Inhibitors |
| | | Anti-CXCL8 mAb |
| | | ABX-CXCL8 |
| | | CXCR2 antagonists |
| | | SCH527123 |
| | | SB-656933 |
| | | GSK-1325756 |
| | | CCR2 antagonists |
| | | INCB-8696 |
| | | INCB-3284 |
| | | INCB3344 |
| | | NIBR-177 |
| | | GSK-1344386B |
| | | CCX-140 |
| | | JNJ-27553292 |
| | | SKL-2841 |
| | | BMS-741672 |
| | | PF-04634817 |
| | | CXCR3 antagonists |
| | | AMG-487 (T-487) |
| | | CX3CL1 antagonists |
| | | FKN-AT |
| | | F1 |
| Phosphatidylinositol | Inflammatory cell response | LY294002 |
| 3 kinase (PI3K) | markers | Small-molecule |
| | | inhibitors of PI3Kc and d |
| | | TG100-115 |
| | | AS252424 |
| | | AS605240 |
| Lung Airway | Spirometry | b2-agonists |
| Response | Imaging | Long-acting |
| | Lung function | antimuscarinic agents |
| | Molecular causal correlations | Methylxanthines |
| | with lung function | |
| Systemic | SAA1 | Statins |
| Inflammation | CRP | ACE inhibitors |
| Acute Phase Reactant | Pentraxin family, PTX3 | Lipid lowering drugs |
| | COX-2 | Anti-infectives |
| | ST2 (IL1RL1) | Antibiotic classes |
| | TNFalpha | Macrolides |
| | IL-6 | Erythromycin |
| | Erythrocyte Sedimentation Rate | Clarithromycin |
| | Cathepsin family, - S, -G | Roxithromycin |
| | PCT | Azithromycin |
| | sTREM1 | Immunolides |
| | MRproADM | EM703 |
| | PRG4 | EM900 |
| | Hyaluronin (HA) | CSY0073 |
| | | CEM-101 |
| | | Synthetic boundary |
| | | lubricants |
| AGE | sRAGE | PPAR agonists |
| | HMGB1 | PEDF therapy |
| | S100A8/A9 (Calprotectin) | |
| | IL1beta-HMGB1 complex | |
| | TNFa | |
| | PPARgamma | |
| | Alpha PEDF (SERPIN) | |
| NF-kB | p65 | Corticosteroids, |
| mPhage | HDAC1/2 (activity) | Glucocorticoid |
| | IL1alpha | TNF |
| | IL1beta | IL1 inhibitors |
| | IL1RA | Theophylline |
| | TNFa | NF-kB Inhibitors |
| | MCP-1 | IKK inhibitors |
| | GM-CSF | IMD-0354 |
| | CCL18 | IMD-0560 |
| | | BMS-345541 |
| | | SC-514 |
| | | ACHP |
| | | Bay 65-1942 |
| | | AS602868 |
| | | PS-1145 |
| | | NF-kB “decoy” |
| | | oligonucleotides |
| | | Antisense and small |
| | | interfering RNA (siRNA) |
| | | TNF inhibitors |
| | | Humanised monoclonal |
| | | antibodies to TNF-a |
| | | Infliximab |
| | | Adalimumab |
| | | Certolizumab pegol |
| | | Golimumab |
| | | Humanised monoclonal |
| | | antibodies to soluble |
| | | TNF-a receptors |
| | | Etanercept |
| | | TACE inhibitors |
| | | PKF 242-484 |
| | | PKF 241-466 |
| | | Inhibitors of TNF-a |
| | | production |
| | | Antisense |
| | | oligonucleotides against |
| | | TNF-a mRNA |
| | | Oral steroids |
| | | Methylprednisolone |
| | | prednisolone |
| | | Prednisone |
| | | Inhaled Steroids |
| | | beclomethasone |
| | | budesonide |
| | | flunisolide |
| | | fluticasone |
| | | mometasone |
| | | Combination Steroids: |
| | | budesonide and |
| | | formoterol |
| | | fluticasone and |
| | | salmeterol |
| | | vilanterol and |
| | | fluticasone |
| | | Steroid enhancers |
| | | Activation of HDAC2 |
| | | Theophylline |
| | | Curcumin |
| | | Resveratrol |
| | | Inhibitors of P- |
| | | glycoprotein |
| | | Inhibitors of MIF |
| cAMP regulation | cAMP | Metformin |
| PDE4 Inhibition | PGE2 | Oral PDE4 inhibitors |
| | VitD forms | Roflumilast |
| | GM-CSF | ELB353 |
| | MUC5AC | Revamilast |
| | TNFa | MEM1414 |
| | IL12 | Oglemilast |
| | LTB4 | OX914 |
| | IL10 | BLX-028914 |
| | Superoxide | ASP3258 |
| | LTC4 | TAS-203 |
| | CD11b | ZI-n-91 |
| | IgE | NIS-62949 |
| | IL4 | NCS 613 |
| | IL5 | Tetomilast |
| | IL13 | Inhaled PDE4 inhibitors |
| | IL2 | GSK256066 |
| | IFN | SCH900182 |
| | | Compound 1 |
| | | Tofimilast |
| | | AWD12-281 |
| | | UK500001 |
| | | PDE3/4 inhibitors |
| | | RPL554 |
| | | PDE4/7 inhibitors |
| | | TPI 1100 |
| Adipokines | Adiponectin | Targeted biologics |
| (composition) | Leptin | |
| Extra Cellular | GSH | Thiol compounds |
| Remodeling | Anti-oxidant enzymes | N-acetyl-L-cysteine |
| REDOX | Thioredoxins | (NAC) |
| | Thioredoxin reductase | N-acystelyn (NAL) |
| | Glutaredoxins | N-isobutyrylcysteine |
| | Glutathione reductase | (NIC) |
| | Peroxiredoxins | Glutathione esters |
| | | S-carboxymethylcysteine |
| | | (carbocysteine) |
| | | Erdosteine |
| | | Fudosteine |
| | | Thioredoxin |
| | | Procysteine |
| | | Ergothioneine |
| | | Inducers of glutathione |
| | | biosynthesis (Nrf2 |
| | | activators) |
| | | Antioxidant vitamins |
| | | (vitamin A, E, C) |
| | | b-carotene |
| | | CoQ10 |
| | | Polyphenols |
| | | Curcumin |
| | | Resveratrol |
| | | Quercetin |
| | | Green tea catechins |
| | | Nitrone spin traps |
| | | NXY-059 |
| | | STANZ |
| | | Porphyrins |
| | | SOD and glutathione |
| | | peroxidase |
| | | mimetics |
| | | M40419 |
| | | M40403 |
| | | M40419 |
| | | Ebselen |
| | | Lipid peroxidation and |
| | | protein carbonylation |
| | | inhibitors/blockers |
| | | Edaravone |
| | | Lazaroids |
| Immune Response | Total IgG | IgC replacement |
| and | IgG subtypes (e.g. IgG1, IgG2) | Anti-IgE |
| Complement Pathways | IgE | Omalizumab |
| | IgA | Anti mucousals |
| | Beta-defensin-2 | Complement inhibitors, |
| | Siglec-7 | C1-INH (SERPING1), |
| | Siglec-8 | endogenous and |
| | Siglec-14 | recombinant forms |
| | C1 | C3, C5 inhibitors |
| | C1q | Anti-factorD |
| | C1qr2s2 | |
| | C1qBP | |
| | CTRP(1, 3, 5, 9, 15), otherwise | |
| | known as C1qTNF(1, 3, 5, 9, 15) | |
| | C3 | |
| | C3a | |
| | iC3b | |
| | C5 | |
| | C5a, b | |
| | Mannose Binding Lectin (MBL) | |
| Th2 activation and | IL2 | Anti-IL4: |
| regulation | IL4 | Pitrakinra |
| Th1 cell activation | IL5 | Anti-IL4Rα/IL13Rα1 |
| and regulation | IL6 | Dipulimab |
| Th17 cell activation | IL10 | Anti-IL5: |
| and regulations | IL12 | Mepolizumab |
| | IL13 | Reslizumab |
| | IL17 (IL17A, IL17F, IL17A/F) | Targeted at IL5 |
| | IL18 | effector cells: |
| | IL18BP | Benralizumab |
| | IL21 | Anti IL6: |
| | IL23 | Sirukumab |
| | IL25 | Tocilizumab |
| | IL27 | Anti-IL13 (periostin |
| | Periostin | CDx): |
| | IgE | |
| | IFN-gamma | |
| | TNF | |
| | CD40L | |
| | CD4+ cells | Lebrikizumab |
| | CD8+ cells | Tralokinumab |
| | ST2, IL1RL1 | Anti-IL12/23: |
| | | Ustekinumab |
| | | Anti-IgE: |
| | | Omalizumab |
| | | Anti-IFgamma: |
| | | AMG 811 |
| | | Anti TNF: |
| | | Etanercept |
| | | Infliximab |
| | | Adalimumab |
| | | Golimumab |
| | | Certolizumab |
| | | Anti-ST2 mab |
| Interstitial Lung | KL-6 | Transplant |
| Disease - general | CC16 | |
| | SP-D | |
| | SP-A | |
| | YKL-40 | |
| | CCL18 | |
| | CCL2 | |
| | CXCL10 | |
| | CXCL12 | |
| | MMP-7 | |
| | MMP-9 | |
| Idiopathic Pulmonary | KL-6 | Transplant |
| Fibrosis | SP-D | Pirfenidone |
| | SP-A | |
| | VEGF | |
| | MMP-7 | |
| | LOXL2 | |
| | Periostin | |
| | Fibrocytes | |
| | CCL18 | |
| | YKL-40 | |
| | IL8 | |
| | ICAM-I | |
| | Sema7a | |
| | CD28 | |
| | anti-HSP70 | |
| | BLyS | |
| | CXCL13 | |
| | MUC5B | |
| | TOLLIP | |
| CVD/CHF, pulmonary | BNP | Anticoagulants and |
| thrombosis, renal | -NT-proBNP | thinners |
| decline | ANP | Cumadin |
| | NT-proANP | Warfarin |
| | sST2 | Heparin |
| | Lp-PLA2 | Heparanoids |
| | D-Dimer | Hymecromone |
| | Lp(a) | Statins (also ox-lipid |
| | Fibronectin | reducers) |
| | Cystatin C | Lovastatin |
| | Creatinine | Simvastatin |
| | Hyaluronin (Acid) | Pravastatin |
| | HABP (PHTN) | Atorvastatin |
| | | Fluvastatin |
| | | Rosuvastatin |
| | | Additional Lipid lowering |
| | | drugs |
| | | PPAR agonists |
| Hypertension | Blood Pressure | Thiazide diuretics |
| | | Beta blockers |
| | | Angiotensin-converting |
| | | enzyme (ACE) inhibitors |
| | | Angiotensin II receptor |
| | | blockers (ARBs) |
| | | Calcium channel |
| | | blockers |
| | | Renin inhibitors |
| | | Alpha blockers |
| | | Alpha-beta blockers |
| | | Central-acting agents |
| | | Vasodilators |
| | | Aldosterone antagonists |
| Diabetes | Insulin and insulin related | Metformin |
| | pathways | TZDs |
| | hA1c | Pioglitazone |
| | Resistin | Rosiglitazone |
| | PPARgamma | |
| Asthma | YKL-40 | Inhaled corticosteroids |
| Allergy | Periostin | LABAs |
| | IL13 | Cromolyn and |
| | CLCA1 | Theophylline |
| | | Leukotriene Modifiers |
| | | Immunomodulators |
| | | Anti-IL13 |
| | | anti-IL5 |
| | | SABAs |
| | | Montelukast |
| | | Immunomodulators |
| | | Oral corticosteroids |
| GI Disorders | FeNO - breath | Protein Pump Inhibitors |
| IBD | ECP | Omeprazole |
| GERD | Neutrophin 3 | Pantoprazole |
| | BDNF | Esomeprazole |
| | Nerve Growth Factor | Lansoprazole |
| | 8-Isoprotane | Rabeprazole |
| | Prostaglandin D2 | Dexlansoprazole |
| | IL4 | Rabeprazole sodium |
| | IL6 | Pantoprazole sodium |
| | IL15 | Esomeprazole |
| | IFN-gamma | magnesium |
| | Fibrinogen | Omeprazole magnesium |
| | Pepsin | Naproxen/Esomeprazole |
| | Mast cell tryptase | Esomeprazole sodium |
| | Elastase | Omeprazole/Bicarbonate |
| | SPA | ion |
| | SPD | Vedolizumab (a4b7) |
| | Lactate Dehydrogenase | |
| Osteoporosis | Serum total alkaline | Antiresorptive |
| | phosphatase | medications |
| | Serum bone-specific | Anabolic drugs |
| | alkaline phosphatase | |
| | Serum osteocalcin | |
| | Serum type 1 procollagen | |
| | (C-terminal/N-terminal): | |
| | C1NP or P1NP | |
| Composition | Lipoprotein (e.g. high density | Benzafibrate |
| | lipoprotein (HDL) and low | Fenofibrate |
| | density lipoprotein (LDL)) | Glitazones |
| | Apolipoprotein (e.g. SAA) | Glimepiride |
| | Adipokines | Angiotensin converting |
| | Leptin | enzyme inhibitors |
| | Adiponectin | Angiotensin receptor |
| | | blockers |
| Lung Cancer | CA19.9 | Anti-angiogenesis |
| | CEA | (Bevacuzimab) |
| | CA125 | Inhibitors of EGFR |
| | IGF-1R | Tyrosine Kinase |
| | IGFBP1-6 | Inhibitors |
| | Phosopho and total AKT | Mabs against EGFR |
| | HDAC | Cetumximab |
| | cMET | Nimotuzumab |
| | proteasome markers | Gefitinib |
| | p21 | Erlotinib |
| | p53 | Inhibitors of VEGF |
| | p27 | Sorafenib |
| | NF-kB | Aflibercept |
| | p65 | Inhibitor of EML4-ALK |
| | Bcl-xL | (never/light smokers |
| | Bcl-2 | subpopulation) |
| | | Figitumumab (IGF-1R) |
| | | Everolimus (mTOR) |
| | | HDAC inhibitors |
| | | Benzamides |
| | | Cyclic tetrapeptides |
| | | ARQ 197 (cMET inh) |
| | | Onartuzumab |
| | | (Metmab) |
| Neuro/Adrenal | Adenosine | CGS21680 |
| response | Prostacyclin | ATL146e |
| Vasodilation | cAMP | UK371, 104 |
| Adenosine (Caffeine) | Nitric Oxide | GW328267X |
| | EDHF | Regadenoson |
| | Prostaglandin E2, D2, I2 | 2-(cyclohexylethylthio)- |
| | Natriuretic peptides | AMP |
| | VIP | |
| | Substance P | |
| | Interstitial lactic acid | |
| | PAF (platelet activating factor) | |

In some cases, a biomarker produces non-monotonic distribution of incidence rate. The non-monotonic distribution can be a “U” or “J” shaped distribution. Examples of biomarker which can product non-monotonic distribution of biomarker importance can include IgA, IgG, IgE, leptin, adiponectin, HNL, Neutrophil elastase, Resistin, advanced glycation end products (AGE) and associated receptors (RAGE) and soluble receptor forms (sRAGE), Growth Differentiating Factor 15 (GDF 15), proANP, C1q, Mannose Binding Lectin (MBL), PTX3, D-Dimer, Cystatin C, Cathepsin, YKL-40 PF4, and RANTES. In some cases, at least one biomarker of the plurality of biomarkers produces a non-monotonic distribution of biomarker importance.

In some cases, the methods comprise detection of an autoantibody specific for a biomarker described herein. Indications of autoantibodies have been noted in COPD. Noxious exposure to cigarette smoke or the like can: 1) initially attract a high level of inflammation and immune response to the lung in response to insult, and 2) provide the environment for oxidation and modification the multiplicity of cells, proteins, proteases and endogenous regulators and mediators of these processes. Autoantibodies formed against inhibitors and repair related molecules, for example in the complement cascade regulation, coagulation and fibrinolysis pathways can lead to dysfunction, hyper- and hypo-responsiveness, and subsequent organ and tissue damage. Autoantibodies against phosopholipids inducing antiphospholipid syndrome like response and/or associate increased arterial and/or venous vascular embolism conditions, alpha2macroglobulin, clotting factor VIII, serine protease inhibitors in general, more specifically A1AT, PAI-1 or -2, C1-inh, complement cascade components, C1q, Pentraxin 3, factor H, Mannose Binding Lectin, or the like in some imbalanced combinations can all result in dysfunctional response and repair. Restriction of some or all of the associated processes can result in more aggressive innate (unrecognized) response and clearance leading to increased functional tissue loss and overall organ damage.

Antibodies for Biomarker Detection

In some cases, the methods comprise contacting a biological sample from an individual with a plurality of antibodies. The plurality of antibodies can be bound to a solid support. The solid support can be a microplate or a bead. In some cases the solid support comprises silica. The bead can be a magnetic bead. The plurality of antibodies can target a plurality of target biomarkers. Each antibody in the plurality of antibodies can have specificity to a single target biomarker. If a target biomarker is present in the biological sample, the target biomarker can bind to its corresponding capture antibody. In some cases, the plurality of antibodies comprise a plurality of capture antibodies and a plurality of detection antibodies.

The plurality of antibodies can be bound, or conjugated, to a detectable label. The detectable label can be a a fluorescent label, an enzymatic label, or a small molecule label. The fluorescent label can be a fluorophore. The fluorophore can be a xanthene, a cyanine, a squaraine, a naphthalene, a oumarin, an oxadiazole, an anthracene, a pyrene, an oxazine, an acridine, an arylmethine, a tetrapyrrole, or derivatives thereof. Examples of xanthene derivatives include, but are not limited to, fluorescein, rhodamine, Oregone green, eosin, and Texas red. Examples of cyanine derivatives include, but are not limited to, cyanine, indocarbocyanine, oxacarbocyanine, thiacarbocyanine, and merocyanine. Examples of squaraine derivatives include, but are not limited to, Seta, SeTau, and Square dyes. Examples of oxadiazole derivatives include, but are not limited to, pyridyloxazole, nitrobenzoxadiazole, and benzoxadiazole. Examples of anthracene derivatives include, but are not limited to, anthraquinoes. Examples of a pyrene derivative includes, but is not limited to, cascade blue. Examples of oxazine derivatives include, but are not limited to Nile red, Nile blue, cresyl violet, and oxazine 170. Examples of acridine derivatives include, but are not limited to proflavin, acridine orange, and acridine yellow. Examples of arylmethine derivatives include, but are not limited to auramine, crystal violet, and malachite green. Examples of tetrapyrrole derivatives include, but are not limited to porphin, phthalocyanine, and bilirubin. The enzymatic label can be an enzyme. The enzyme can be alkaline phosphatase, horseradish peroxidase, β-galactosidase, or glucose oxidase. The small molecule label can be a hapten. The hapten can be oxazole, pyrazole, thiazole, nitroaryl, benzofurazan, triterpene, urea, thiourea, rotenoid, coumarin, cyclolignan, heterobiaryl, azoaryl, benzodiazepine, or a derivative thereof. In some cases, the detectable label is biotin.

The plurality of antibodies can comprise monoclonal antibodies, polyclonal antibodies, or a combination thereof. In some cases, when a plurality of capture antibodies and a plurality of detection antibodies are used, the configuration of the plurality of capture antibodies and detection antibodies are described in Table 2. In one example, four capture antibodies and four corresponding detection antibodies can be used to detect RANTES, CRP, A1AT, and MMP-9, wherein the configuration of the capture antibodies and detection antibodies are described in Table 2.

A specific configuration of antibodies for use in the methods described herein can be chosen based on standard analytical performance specifications. Standard analytical performance specification can include, but are not limited to, limits of detection, upper and lower limits of quantification and dynamic range with respect to the measured biomarkers, reproducibility as determined by precision in the <15% CV range, interfering substances as measured by spike recoveries 80-120% within a selection of samples including disease samples, accuracy as determined by dilution linearity and parallelism, compliance of assay run and process controls using specific pooled disease and normal samples, or a combination thereof.

TABLE 2
Possible antibody configurations for use in a detection assay

| Biomarker | Capture | Detection |
|---|---|---|
| Adiponectin | Mono | Mono |
| A1AT | Poly | Poly |
| C1Q | Poly | Poly |
| Cathepsin S | Poly | Poly |
| CC16 | Mono | Poly |
| CRP | Mono | Poly |
| Cystatin C | Mono | Mono |
| D-DIMER | Mono | Mono |
| Eotaxin | Mono | Poly |
| Fibrinogen | Mono | Mono |
| Fibronectin | Poly | Poly |
| GDF-15 | Mono | Poly |
| HNL | Mono | Poly |
| IgA | Poly | Poly |
| IgE | Poly | Poly |
| IgG | Poly | Poly |
| IgG1 | Poly | Mono |
| IgG2 | Poly | Mono |
| IL-6 | Mono | Mono |
| Leptin | Mono | Mono |
| MBL | Poly | Poly |
| MCP-1 | Mono | Poly |
| MMP-9 | Poly | Poly |
| Neutrophil | Poly | Poly |
| Elastase | | |
| PARC | Poly | Poly |
| PCT | Poly | Poly |
| Pentraxin 3 | Mono | Poly |
| PF4 | Poly | Poly |
| Pro-NT ANP | Poly | Mono |
| P-Selectin | Poly | Poly |
| RANTES | Mono | Mono |
| Resistin | Poly | Poly |
| SAA | Mono | Mono |
| sRAGE | Poly | Poly |
| Timp1 | Mono | Poly |
| YKL-40 | Poly | Poly |

(mono = monoclonal; poly = polyclonal)

Kits

Also provided are kits that at least include at least one antibody for detecting the at least one biomarker described herein. The at least one antibody can comprise at least one capture antibody, at least one detection antibody, or a combination thereof. The kit can further comprise instructions for how to use the plurality antibodies to carry out one or more of the methods described herein. In one aspect, the kit comprises a plurality of antibodies. The plurality of antibodies can detect at least 2, 3, 4, 5, 6, 7, 8, 9, or more than 10 biomarkers. The kit can further comprise a solid support. The at least one antibody can be pre-bound to the solid support. Alternatively, the at least one antibody can be packaged separately from the solid support. The kit can further comprise the at least one capture antibody packaged separately from the at least one detection antibody (e.g., each are present in a separate container).

The kit can further comprise an additional reagent. The additional reagent can be a buffer. The additional reagent can comprise a component to improve the binding of at least one biomarker to the at least one antibody. The additional reagent can comprise a component to improve the stability of the at least one antibody. The additional reagent can be a goat serum protein, a bovine serum albumin, trehalose, sucrose, a chelating agent, or a combination thereof. The additional reagent can be packaged with the at least one antibody. Alternatively, the additional reagent can be packaged separately from the at least one antibody.

The instructions for using the at least one antibody as discussed above are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub-packaging) etc. In one aspect, the instructions are present as an electronic storage data file present on a suitable computer-readable storage medium, e.g., a digital storage medium, e.g., a portable flash drive, a CD-ROM, a diskette, etc. The instructions may take any form, including complete instructions for how to use the systems and devices, or as a website address with which instructions posted on the Internet may be accessed.

Biomarker Descriptions and Relevance

Whether or not a combination of biomarkers is informative can depend on clinical and demographic measures of a population or individual selected for analysis. The combination of biomarkers can be any combination of biomarkers described herein, including, but not limited to those molecules described in Table 1 and further described below. The biomarker can be the molecule itself, an autoantibody to the molecule, a receptor of the molecule, a complex comprising the molecule, or any combination thereof.

Alpha-1 Antitrypsin (Serpin A1, A1AT) is a member of the serpin superfamily. A1AT is a protease inhibitor that can be secreted into circulation by the liver and can function primarily to protect tissues such as lung from the action of neutrophil elastase released during an inflammatory response. Neutrophil Elastase can be implicated in the pathogenesis of COPD. A1AT can be an inhibitor of neutrophil elastase. The levels of A1AT in the blood stream can increase in response to acute inflammation such as that seen in COPD. The protease/anti-protease imbalance can be a driver in lung damage associated with COPD progression.

Alpha-2-Macroglobulin (A2M) is a 720KD plasma protein found in blood. A2M can primarily be synthesized in the liver, but also can be locally made by microphages and fibroblasts. A2M can act as a protease inhibitor and is thought to have a broad specificity that includes serine, cysteine, aspartic, and MMP. A2M can function by binding and sequestering the protease, wherein the bound protease can still cleave their target peptides. A2M may also function as a transporter of cytokines and growth factors. A2M can be considered an acute phase reactant and has been shown to be elevated in COPD. A2M levels, when combined with clinical features, may allow for better predictions of future severe exacerbations

Adiponectin (Acrp30) is an adipokine secreted by adipocytes. Adiponectin sequence show similarity to the complement C1q factors, while structurally appears to fall in the TNF-alpha family. Adiponectin can be implicated in both metabolic regulation as well as inflammation. Adiponectin can have anti-inflammatory effects in metabolic disorder (diabetes, obesity etc.), while exhibiting pro-inflammatory effects in non-metabolic disorder such as RA. Adiponectin can play a role in angiogenesis and tissue remodeling by binding to different growth factor and inhibiting their function. COPD is a disease that can include both systemic inflammation as well as tissue re-modeling in the lung. The level of Adiponectin can be associated with BMI. Elevated BMI can lead to a better prognosis for COPD patients, suggesting that in some instances there can be a link between the functions of adiponectin and COPD progression and prognosis. Moreover, elevated levels of serum adiponectin in COPD can be associated with decline in lung function.

Complement component 1q (C1q) is the first subcomponent of the C1 complex of the classical pathway of complement activation. Functions of C1q can include antibody-dependent and independent immune functions, which can be considered to be mediated by C1q receptors present on the effector cell surface A lack of C1q can be a sign of immune deficiency. With low C1q, the alternate complement pathway can be engaged, increasing the severity of subsequent inflammation. High C1q level can be associated with rapid aging. The C1q molecule can actively engage the Wilt pathways and increased cellular senescence resulting in the high turnover of a stressed immune response consuming otherwise healthy and/or functional tissue.

Calprotectin is a complex of S100A8/S100A9 and can function in part to chelate and sequester manganese and zinc. Calprotectin can be involved in the innate immune response, possibly via activation of TLR-4. Calprotectin can be found in high levels in neutrophils and can be secreted in response to inflammation. Calprotectin can be elevated in disease associated with chronic inflammation. Calprotectin can also exhibit anti-microbial properties due to the ability to sequester manganese and zinc. Calprotectin can be elevated in COPD and can be associated with all-cause mortality in COPD. Levels of calprotectin can be associated with neutrophilic inflammation in uncontrolled asthma. As subtypes of COPD have been characterized as neutrophil driven, elevated levels of calprotectin can indicate neutrophil activation in response to an inflammatory event.

Cathepsin S is expressed by antigen presenting cells and is a lysosomal cysteine protease. It can function to degrade antigenic protein for antigen presentation. Cathepsin S can function as an elastase and maintain activity at neutral pH. Cathepsin S activity can be tightly regulated by its specific inhibitor, Cystatin C. Circulating levels of Cathepsin S, as well as its inhibitor Cystatin C, can be significantly elevated in COPD. In some instances, Cathepsin S levels can be negatively associated with airflow limitation as well as severity of emphysema.

Club cell 16 protein (uteroglobin, club cell secretory protein, CC10, CC16) is a member of the secretoglobin family of proteins. CC16 can be expressed in Club Cells of the lung bronchioles. CC16 is an anti-inflammatory protein and can have immunoregulatory properties that include inhibition of cell migration and T Cell differentiation. Levels of CC16 can be inversely correlated with COPD and lower levels of CC16 in smokers can be associated with progression to COPD. High levels of CC16 can be protective for development of COPD. CC16 augmentation therapy can be suggested for at risk smokers and COPD patients.

C-Reactive protein (CRP) is a pentraxin family member and can be an acute-phase protein produced and secreted by the liver. CRP can increase in response to either acute or chronic inflammation. CRP levels can increase in response to microphage and adipocyte secretion of IL-6 and other cytokines and lead to activation of the complement pathway. CRP levels can increase in response to infection, inflammation and tissue damage. As an acute-phase protein, levels of CRP can rise rapidly upon inflammation and thus CRP can function as a biomarker of active inflammation. Moreover, CRP can have a relatively short half-life and thus can also be used to monitor resolution of the inflammatory insult. In COPD, exacerbation of the levels of CRP can be associated with bacterial etiology in that viral etiologies resulting in a reduced elevation of CRP. However, as an acute-phase protein, elevated levels of CRP can be indicative of systemic inflammation and may not indicate etiology.

Cystatin C can be expressed in nearly all tissues of the body. Cystatin C can be used as a biomarker for kidney function. Cystatin can function as a inhibitor of cysteine proteinases and as such can prevent breakdown of extracellular matrix. Cystatin C can inhibit the enzymatic activity of the cysteine proteinase, Cathepsin S. Both Cystatin C and cathepsin S can be coordinately expressed to better regulate the proteinase activity. In COPD, damage to the lungs can lead to remodeling and breakdown of the extracellular matrix. Cystatin C can be elevated in COPD along with Cathepsin S. Moreover, the levels of Cystatin C can be correlated with stable COPD and negatively correlated with FEV1% predicted. Progression of COPD can be associated with an imbalance in the proteinase-anti-proteinase ratio. As such, in COPD, the ratio of Cathepsin S/Cystatin C can be related to decline in lung function.

D-dimer can be a degradation product of fibrin. D-dimer can be produced through fibrinolysis, or degradation of a blood clot. D-dimers can be present in the blood upon activation of the coagulation pathway. Clinically, elevated levels of D-dimer can be associated with pulmonary embolism as well as other thrombolytic pathologies. In addition, elevated D-dimer can be associated with active inflammation. D-dimer can be elevated in COPD and further elevated during exacerbation. The levels of D-dimer can be an index for severity of COPD exacerbation.

Eotaxin-1 (CCL11) is part of the CC chemokine family. Eotaxin-1 can induce chemotaxis in eosinophils. Due to this specificity, high levels of Eotaxon-1 can be indicative of activated eosinophils. Airway eosinophilia can be a hallmark of asthma, and recent studies have described a similar phenotype in a sub-group of COPD patients without asthma (called eosinophilic COPD). Airway eosinophilia can be linked to increased risk of exacerbation. Therapies available for asthma that target eosinophils can show some efficacy in COPD. Eotaxin-1 can be implicated in the allergic response. Eosinophil levels can be implicated in COPD progression. In stable COPD, Eotaxin-1 levels can be reduced relative to healthy controls, and can be correlated with FEV1%. Eotaxin-1 can be elevated with COPD progression and can be significantly elevated in in rapid decliners.

Eosinophil Cationic Protein (ECP, Ribonuclease 3) is a basic protein localized to the granule matrix of eosinophil and can be released upon degranulation. ECP can be elevated during inflammation and in asthma. ECP can induce apoptosis of cells, for example bronchial epithelial cells. Anti-IgE treatment of ACOS patients can lead to a decrease in ECP. ECP can be elevated in COPD and during exacerbation. ECP can be linked to asthma. ECP can be associated with eosinophilic driven response.

Fibrinogen can be synthesized and secreted by the liver. Fibrinogen can circulate in blood, and upon tissue damage, injury or infection it can be converted to fibrin that results in development of a blood clot. COPD can be associated with tissue damage and COPD exacerbations can result from pulmonary infections. Fibrinogen is an acute-phase protein and as such its levels can increase during systemic inflammation. Plasma fibrinogen level can be significantly elevated in COPD and these elevated levels can be associated with increased mortality. Fibrinogen is one of the few FDA approved blood based biomarkers for COPD and can be used as an end-point measurement in therapeutic drug trails.

Fibronectin exists as part of the extracellular matrix as a polymeric network and a soluble dimer in plasma. Fibronectin can be involved in numerous functions, including cell adhesion and migration, morphogenesis and tissue/wound repair. Alteration in the extracellular matrix in the lung can be a key feature of COPD. The ratio of soluble fibronectin to the inflammatory marker CRP can be associated with all-cause mortality in COPD.

Growth Differentiation Factor 15 (GDF-15, MIC-1, Microphage Inhibitory Factor 1) is a member of the TGF-beta superfamily. GDF-15 can be cardioprotective via its inhibition of platelet activation. In COPD, levels of GDF-15 can be associated cardiovascular risk. Levels of GDF-15 can be correlated with CRP levels and can be described to be elevated in acute exacerbation of COPD. Elevated levels of GDF-15 can be independently linked to frequent rates of COPD exacerbation as well as elevated mortality.

Human neutrophil lipocalin (HNL, NGAL, Lipocalin 2) is a component of neutrophil granules. HNL can be a marker of neutrophil activation. HNL can be considered an acute phase protein and can be involved with the innate immune response to infection. HNL can behave differently in asthma versus COPD. HNL can be elevated in healthy smokers as compared to never-smokers. Neutrophil levels, and thus level of HNL, can be associated with severity of COPD. Examination of HNL and eosinophilic markers (ECP) during glucocorticoid treatment of asthma and COPD can indicate that treatment of asthmatics result in a decrease in inflammation as measured by eosinophilic markers, while having no anti-inflammatory effect on COPD patients, suggesting that some groups of COPD patients may be resistant to the anti-inflammatory effect of glucocorticoids. Neutrophilic driven COPD pathology can be monitored by HNL.

High Mobility Group 1 (HMGB1, Amphoterin) is a DNA binding protein which can be involved in regulation of chromatin structure and implicated in transcriptional regulation. HMGB1, an alarmin, can also be involved in the inflammatory response, and upon release from macrophages, monocytes and dendritic cells can function as a pro-inflammatory cytokine. HMGB1 activated cytokine can release from microphages via interaction with TLR4. HMGB1 can bind and sequester sRAGE. HMGB1 can be elevated in COPD and can change dynamically in conjunction with sRAGE during COPD exacerbation and recovery.

Immunoglobulin A (IgA) is involved in immunity and secretory IgA (sIgA) can be involved in mucosal immunity. sIgA can be involved in creating a mucosal barrier for bacterial infections. In some COPD there can be a deficiency of sIgA in the small airways, which can lead to a greater susceptibility to infections in the lung. Deficiency in sIgA can lead to increased risk of exacerbation due to increased risk of bacterial infections of the lung.

Immunoglobulin E (IgE) is synthesized and secreted by plasma cells. One of the major roles of IgE can be defense against parasites. IgE can be implicated in type 1 hypersensitivity associated with allergic reactions. IgE can function by binding to Fc receptors on mast cell and basophils. Interaction of the IgE with basophils can promote the release of type 2 cytokines. While asthma and COPD can exhibit similar phenotype and can both exhibit an exacerbation phenotype, the etiology can be distinct. Specifically, the decline in lung function for COPD can be sustained while lung function can be reversible in asthma. Moreover, the inflammatory response in asthma can be different than the inflammatory response for COPD, as asthma can be eosinophilic while COPD can be neutrophilic. COPD and asthma can require different therapeutic interventions. Further complicating this issue is presence of individual with asthma/COPD overlap syndrome. Upon exacerbation, the treatment associated with COPD versus one mediated via an allergic asthmatic response can require distinct therapeutic interventions. In some instances, the level of IgE distinguishes the etiology and guides therapy. Allergic reactions mediated by IgE can contribute to the severity of COPD. Allergic reactions to tobacco related compounds, tobacco smoke being one of the major culprits, can be implicated in the development of COPD. Elevated levels of IgE can be useful in distinguishing different etiologies of COPD.

Immunoglobulin G (IgG) is the predominant class of antibody and can constitute nearly 2/3 of serum antibodies. IgG can be synthesized and released by plasma B cells. IgG can be a component of humoral immunity and protects the body from pathogens. IgG can be produced during the secondary immune response and considered part of the adaptive immune process. IgG's can be composed of four subclasses: IgG1, IgG2, IgG3 and IgG4. The different subclasses of IgG can differ in their abundance (for example, IgG1>>IgG2>IgG3>IgG4) as well as their ability to activate the complement pathways (for example, IgG3>IgG2>IgG3, IgG4 does not). IgG's can differ in their affinity of the Fc receptors on phagocytic cells and their half-life. For example, IgG3 can have the shortest half-life. The different subclasses of IgG may be expressed temporally during the immune response. There may be a link between immune deficiency and COPD, specifically a deficiency of IgG. There may be a correlation between IgG subclasses and risk of exacerbation and hospitalization.

Interleukin 1 beta (IL-1β) is a member of the interleukin 1 family of cytokines. IL-1β can be produced by a variety of cell types and can be an important component of the inflammatory response. IL-1β can be secreted by activated macrophages in a pro-form and subsequently activated by the actions of caspases. IL-1β can be elevated in COPD and indicative of systemic inflammation. Moreover, IL-1β can increase during exacerbation relative to the stable state. The level of IL-1β can be directly proportional to FEV-1. IL-1β can be involved in the innate immune response. For example, upon activation IL-1β can initiate an acute phase inflammatory response. Elevated IL-1β can be associated with bacterial airway infections and bacterial mediated COPD exacerbations. Airway IL-1β can be linked to frequent exacerbations and can be predictive for future events. Anti-IL-1β monoclonal antibody (Canakinumab) has been evaluated as a therapeutic intervention in COPD and is thought to function by reducing systemic inflammation.

Interleukin 5 (IL-5) is a member of the cytokine family. IL-5 can be produced by type 2 T Helper cells, mast cells and eosinophils. IL-5, via its receptor (IL-5Ra), can promote growth of B-cells and modulate eosinophils. As such, IL-5 can be associated with eosinophilic driven response, such as that observed for asthma as well as allergic type reactions. An anti-IL-5 monoclonal antibody can be used therapeutically and demonstrated to ameliorate excessive eosinophilia. Some subclasses of COPD can be eosinophil driven, thus suggesting it as a possible therapeutic target. In addition, IL-5 may be an important molecule in asthma-COPD overlap syndrome.

Interleukin 4 (IL-4) one of the so called Th2 cytokines, can be an important regulator of humeral and adaptive immunity. IL-4 can function similar to IL-13, another Th2 cytokine, by promoting increases in the asthma related periostin. Both IL-13 and IL-4 can have similar affinity for receptors, in that IL-4 can function through IL-13 receptors and vice versa. IL-4 can induce differentiation of Th0 cells in Th2 cells that then secrete IL-4. IL-4 can enhance activated B-Cells, promote differentiation of B-Cells into plasma cells and promote T-Cell proliferation. IL-4 can be important in asthma and allergic response in that it induces production of IgE. IL-4 can inhibit classical activation of macrophages into M1 cells. IL-4 can increase repair associated M2 cells, secretion of anti-inflammatory cytokines that can cause a reduction in inflammation. IL-4 level can be elevated in asthma, COPD and asthma-COPD overlap syndrome. Anti-IL-4 monoclonal antibody (dupilumab) has been evaluated in for eosinophilic asthma. Use of Omalizumab (Anti-IgE monoclonal antibody) in ACOS can be associated with a decrease in level of IL-4

Interleukin 6 (IL-6) can be involved in a broad range of effects, including the acute phase reaction and inflammation. As an important mediator of inflammation, IL-6 can be associated with numerous pathological conditions associated with chronic inflammation, including: Obesity & Metabolic syndrome, diabetes, rheumatoid arthritis (RA), inflammatory bowel disease (IBD), and cancer. IL-6, along with several other factors, can be a key component in the acute inflammatory response as well as conditions of chronic inflammation. IL-6 can be an important component in immunity as it can drive the differentiation of B-Cells into IgG-secreting Plasma Cells. COPD is a disease of chronic inflammation, as such, IL-6 can play a role in the pathogenesis of the disease. Serum IL-6 can be elevated in COPD relative to healthy controls. In some cases, levels of IL-6 are associated with disease severity. Moreover, levels of IL-6 can be associated with exacerbation and can have prognostic value for mortality.

Interleukin 13 (IL-13) is a cytokine which can be secreted by numerous immune cells, such as Th2, NK T, mast, basophils, and eosinophils. IL-13 can mediate allergic inflammation and has been implicated in asthma. IL-13 can be associated with airway disease and can be demonstrated to induce secretion of MMP's. IL-13 can promote differentiation of goblet cells leading to the production and secretion of Mucin thereby resulting in excessive mucus in the bronchi. IL-13 can be key in the regulation of IgE production and can induce expression of periostin. IL-13 can be elevated in COPD, ACOS and asthma but may not distinguish the different disease. Targeting of IL-13 using monoclonal antibodies (lebrikizumab, tralokinumab) have been looked at for eosinophilic asthma and COPD.

Interleukin 17A (IL-17A) is a pro-inflammatory cytokine which can be produced by activated T cells. IL-17A can be associated with chronic inflammatory diseases such as arthritis and psoriasis. IL-17A can be important for anti-microbial response. IL-17A can promote production of proinflammatory cytokines such as IL-6, chemokines and neutrophil influx. IL-17A can be involved in recruitment of neutrophils, a major driver of COPD and COPD exacerbation. In animal models, anti-IL-17 neutralizing antibodies can result in a reduced recruitment of neutrophils and reduced airway inflammation. IL-17A can be positively correlated with severity of asthma. Th17 mediated inflammation in the airway can be linked to steroid resistance. COPD patients can show an increase in TH17 cells and levels of Th17 cells can be inversely correlated with lung function. In addition, IL-17A can be highly elevated in end-stage COPD.

Interleukin 33 (IL-33) is a member of the IL-1 cytokine superfamily. IL-33 can function via the Il1RL1 (ST2) and Il1RAP and can promote the synthesis and release of Type 2 cytokines. IL-33 can act upon helper T cells, mast cells, eosinophils and basophils. Elevated levels of IL-33 can be associated with asthma and COPD. sST2 can be the soluble form of the Il33 receptor, and can function to scavenge IL-33 and attenuate its function.

Leptin is an adipokine secreted adipocytes. Leptin is a hormone that when secreted can signal the brain that sufficient energy store are available and thus has been termed the satiety hormone. Its primary function is in maintaining energy balance. Due to its role in signaling satiety, leptin can be implicated in obesity and metabolic syndrome. Levels of leptin can be increased with obesity. Leptin levels can be decreased with increased testosterone and can be increased with increased estrogen, indicating difference in energy signaling with gender. In COPD, BMI can impact pathogenesis of the disease. Moderate obesity in COPD can result in a better prognosis regarding disease progression. In addition, leptin can have pro-angiogenic properties and can be important in matrix remodeling by regulating expression of MMP's and their inhibitors (TIMP's). MMP's and TIMP's can be implicated in progression of COPD. Leptin can be involved in innate immunity and can promote the secretion of pro-inflammatory mediators. In COPD, the leptin to adiponectin ratio hcan be prognostic for decline in lung function.

Mannose-Binding Lectin (MBL) is a lectin protein which can be involved in innate immunity. MBL is a member of the C-type lectin superfamily. MBL can be synthesized in the liver in response to infection and can be considered an acute phase protein. MBL can be involved in pattern recognition, and can bind carbohydrates on the surface of pathogens. The binding of MBL to a carbohydrate of a pathogen can lead to activation of complement lectin pathway. MBL can bind apoptotic cells to enhance clearance. MBL deficiency can result in reduced COPD exacerbation. Low levels of MBL can lead to increased infective exacerbation and hospitalization. COPD patients with high MBL levels can be associated with increased survival and not associated with exacerbation frequency. Different levels of MBL (high, intermediate, low) may contribute to diverse outcome in COPD

Monocyte Chemoattractant Protein 1 (MCP-1, CCL2) is a member of the CC chemokine family. Although MCP-1 can be secreted by multiple cell types, monocytes and microphages can represent the major source of MCP-1. MCP-1 can be a chemoattractant that recruit monocytes, memory T cells and dendritic cells to sites of inflammation resulting from tissue injury or infection. MCP-1 can be elevated in COPD as compared to healthy non-smokers and smokers. In COPD, the levels of microphages in the lung can be increased several fold and can be linked to the level of severity, thus the levels of MCP-1 that recruit these cells to the site can be linked to tissue damage, inflammation and progression of the disease.

Matrix metallopeptidase 7 (MMP-7) is a member of the zinc-metalloproteinases. MMP-7 can be involved in degradation of the extracellular matrix (ECM). MMP-7 can be involved in numerous biological processes that include tissue remodeling and repair. MMP-7 can be implicated in arthritic disease progression. MMP-7 can be inhibited by timp-1 and -2. MMP-7 can cleave the pro-peptides and activate MMP-9, another MMP that has been implicated in COPD. MMP-7 can be elevated in IPF. MMP-7 can be a biomarker for IPF. MMP-7 can be elevated in COPD.

Matrix metallopeptidase 8 (MMP-8, Neutrophil collagenase) is a member of the zinc-metalloproteinases. MMP-8 can be expressed in neutrophils and involved in degradation of the extracellular matrix. MMP-8 can be secreted in response to numerous pro-inflammatory cytokines. MMP-8 can be elevated in COPD and IPF. MMP-8 can show a transient increase in the sputum during COPD exacerbation. MMP-8 levels, along with numerous other inflammatory marker, may provide an inflammatory signature that distinguished COPD from NSCLC. MMP-8 levels can differentiate stage 0 COPD from non-symptomatic smoker.

Matrix metallopeptidase 9 (MMP-9) is a member of the zinc-metalloproteinases. MMP-9 can be involved in the degradation of the extracellular matrix, which can lead to tissue damage. MMP-9 mediated degradation of the extracellular matrix can be a component of chronic inflammatory diseases such as COPD. MMP-9 expression can be induced by the pro-inflammatory cytokine IL-1beta. Serum levels of MMP-9 and its inhibitor Timp-1 can be elevated in COPD. The protease-anti-protease imbalance can be a key component of COPD. Moreover, the MMP-9/timp-1 ratio can be diagnostic for early stage COPD. In COPD, serum MMP-9 can be related to severity, and in COPD exacerbation, MMP-9 can be significantly elevated/activated.

Matrix metallopeptidase 12 (MMP-12, macrophage elastase) is a member of the zinc-metalloproteinases. MMP-12 can function to degrade the extracellular matrix. At sites of inflammation, cytokines can induce secretion of MMP-12 from macrophages. Degradation of the ECM during inflammation by MMP-12 can play a key role in pulmonary diseases such as COPD. MMP-12 can be elevated in COPD and in smokers with asthma as compared to healthy smokers. Sputum MMP-12 levels can be associated with emphysema severity as assessed by CT, but not with spirometry. Selective inhibition of MMP-12 can be a therapeutic intervention for COPD.

Myeloperoxidase (MPO) is a peroxidase enzyme that can be produced in neutrophil granulocytes. MPO can be released from neutrophils during degranulation and thus can function as a marker for neutrophil activation. The products of the MPO activity can be anti-microbial. Subclasses of COPD exacerbations can be neutrophilic driven thus elevation of MPO and increased damage of lung tissue could ensue. MPO levels can be increased in stable COPD and can exhibit a further increase upon exacerbation. Smoking can promote elevation of MPO and may provide a measure to predict the progression to COPD.

Neutrophil Elastase: Neutrophil elastase is a serine protease with broad substrate specificity. Neutrophil elastase can be secreted by neutrophils and microphages upon inflammation or infection. Neutrophil elastase can be involved in the response to bacterial infection by degrading protein on the outer membrane of the bacteria. Neutrophil elastase secretion in the lungs during inflammation can lead to destruction of the extracellular matrix resulting in destruction of lung tissue, thereby propagating the disease. The effect of neutrophil elastase can be countered by serpin proteins (eg. A1AT) that can inhibit the enzymatic activity of neutrophil elastase. In COPD, neutrophil elastase can promote degradation of the lung tissue leading to disease progression. The serum levels of neutrophil elastase can be associated with smoking and COPD severity and progression. Modulation of the activity of neutrophil elastase, such as with A1AT augmentation therapy, can be used for therapeutic intervention.

Pulmonary and Activation-Regulated Chemokine (PARC, C-C motif chemokine ligand 1, CCL18, MIP-4) is a member of the CC chemokine family and can be highly expressed in lungs. PARC can be a chemoattractant for naïve T-lymphocytes towards dendritic cells and macrophages. PARC can be involved in both the humeral and the cell-mediated immune response. PARC can be secreted primarily by antigen-presenting cells (dendritic, monocytes and microphages). PARC can be elevated in serum of COPD patients and can be associated with increased risk of cardiovascular hospitalization and mortality. In addition, elevated levels of serum PARC can correlate with COPD exacerbation frequency.

Pro-Calcitonin (PCT) is the peptide precursor of the calcitonin. PCT can be an acute phase protein that can be elevated upon pro-inflammatory stimulation. Elevation of PCT can be associated with inflammation primarily from bacterial origins. PCT is below levels of detection in normal healthy individual and exhibits a pronounce elevation upon infection. PCT can be expressed primarily in the lungs and intestines. Elevation of PCT during acute exacerbation of COPD can be indicative of a bacterial etiology, and as such can be an indicator for antibiotic treatment. PCT can be associated with frequency of exacerbation, perhaps due to a persistent bacterial infection. PCT can be elevated in COPD as compared to healthy controls.

Pentraxin 3 (PTX3) is also known as TNF-inducible gene 14 protein. PTX 3 can be produced and secreted in response to inflammatory signals by numerous cells. PTX3 can be induced by TNF-alpha and IL-1beta. PTX3 can bind to complement C1q and can activate the complement pathway. PTX3 can be involved in the response to microbes and extracellular matrix stability. PTX3 can be an acute phase protein, and the level of PTX3 can rise rapidly under inflammatory condition. PTX 3 can be inversely correlated with COPD severity. In some instances, PTX3 is highly elevated during COPD exacerbation. In some instances, PTX3 exhibits similar patterns and roles as CRP and SAA.

Periostin (OSF-2) can be secreted as an ECM protein. Periostin can be a marker of Th2 inflammatory response asthma. Periostin can be correlated with eosinophilic asthma and eosinophilic COPD. Periostin can be invovled in tissue repair and remodeling. High Periostin levels can be associated with improved lung function following treatment with ICS/LABA. The frequent exacerbator phenotype for COPD can be associated with higher periostin levels as compared to non-frequent exacerbators.

Platelet Factor 4 (PF4, CXCL4) is a member of the CXC chemokine family. PF4 can be secreted by platelets during platelet aggregation. Upon secretion, PF4 can regulate the coagulation cascade. PF4 can bind fibrin and heparin, thereby affecting clot structure. PF4 can play a role in tissue repair and inflammation. PF4 can have chemotactic effects on neutrophils and monocytes. PF4 can form functional complexes with other chemokines (such as RANTES). During acute exacerbation of COPD, abnormal platelet activation can be observed, as such, PF4 can be a potential biomarker for severity of exacerbation.

Pro-peptide of Atrial Natriuretic Peptide (NT-ProANP) can be used as a surrogate measure of production of Atrial Natriuretic Peptide (ANP) dues to its enhanced stability. ANP, a natriuretic peptide hormone, acts on the kidney to promote sodium secretion and maintain extracellular fluid balance. ANP can be produced and secreted by myocytes in the atrial walls. ANP secretion can be stimulated by increased stretching of the atrial walls, which can indicate an increase in blood volume. Levels of ANP can be elevated in COPD, markedly so during exacerbation. ANP can be used as a biomarker for cardiovascular disease. In some instances, cardiovascular disease is a co-morbidity of COPD.

P-Selectin is a member of the selectin family of proteins. P-Selectin is a cell surface protein found on activated platelet and endothelial cells. P-Selectin can be an adhesion protein recruiting leukocyte and neutrophils to a site of injury on the endothelium during inflammation. The soluble form of P-Selectin can be the extracellular domain that is be shed. The levels of both P-Selectin and the soluble form can be associated with platelet activation. COPD patient can have elevated levels of activated/aggregated platelets. The level of activated platelets can be further increased during a COPD exacerbation. Thus, the platelet activation as monitored by P-Selectin levels can be a measure of the risk for cardiovascular risk, a known co-morbidity of COPD.

Regulated on Activation, Normal T Cell Expressed and Secreted (RANTES, chemokine (C-C motif) ligand 5; CCL5) is a chemokine and can be involved in the inflammatory immune response. RANTES can function as a chemoattractant for memory T-Cells and monocytes. RANTES can attract and activate eosinophils. Both neutrophilia and eosinophilia can be associated with COPD. RANTES can be associated with elevated levels of eosinophils and can be elevated with COPD and COPD exacerbation. RANTES can form functional complexes with other chemokines such as PF4 (CXC14). Formation of complexes of RANTES with other chemokines and their action can be involved in the pathogenesis of cardiovascular disease and possibly in COPD.

Resistin is an adipose-derived hormone involved in insulin resistance and thereby thought to be important in obesity and type 2 Diabetes. Resistin can be important for maintaining energy balance and inflammation. Resistin can induce expression of numerous pro-inflammatory cytokines such as IL-6 and IL-1 as well as several proteins involved in the recruitment of leukocytes. Because of its dual role in both inflammation and type 2 diabetes, Resistin can provide a mechanism for the known linkage between inflammation, obesity and insulin resistance. As such, resistin can play a role in chronic inflammation and can be a player in COPD. In COPD, the levels of resistin and insulin can be markedly elevated. Thus, resistin can be involved in the chronic inflammation observed in COPD. In some instances, resistin contributes to insulin resistance observed in some COPD patients.

SAA-1: SAA (Serum amyloid A-1) is an acute phase reactant produce by the liver in response to pro-inflammatory cytokines, such as IL-6 and IL-1beta. Upon induction, SAA can stimulate expression of pro-inflammatory cytokines that include IL-6 and IL-1beta. SAA can be a part of the innate immune response dealing with bacterial infections. SAA can have pro-inflammatory effects, and can activate epithelial cells, neutrophils, monocytes and Th17 cells. SAA can be correlated and behave in a similar fashion to that observed by another acute phase reactant produced by the liver, CRP. However, unlike CRP, SAA can be produced by microphages in the lung. SAA expression can be positively responsive to steroids and can increase during anti-inflammatory steroid therapy, thus, suggesting a possible reason why this type of therapy exhibits poor efficacy in COPD patients. SAA levels can be elevated in COPD and can be elevated during exacerbation. SAA levels can be correlated with the severity of the COPD exacerbation event.

Soluble Receptor for Advanced Glycation End products (sRAGE) is the soluble for form of the Receptor for Advanced Glycation End products (RAGE). RAGE can be expressed at low levels in many tissues. RAGE expression can be upregulated upon interaction with its ligand. Upon activation, RAGE, depending on the context, can activate an array of divergent signaling events that include inflammation, immunity, proliferation, cell adhesion and migration. RAGE can be implicated in numerous pathologies including those associated with chronic inflammation. In the lung, RAGE can be expressed at relatively high basal levels and can be upregulated with pathological condition. HMGB1, upon release form necrotic or inflammatory cells, can function as a pro-inflammatory cytokine and is a ligand for RAGE. sRAGE can function as a decoy for RAGE ligands, including HMGB1, thereby regulating RAGE activation. In COPD, the levels of RAGE and HMGB1 can be elevated, while the levels of sRAGE can be reduced. Upon exacerbation, the levels of sRAGE can be further reduced, which can suggest a role for RAGE in the chronic inflammation associated with COPD. Reduction of plasma sRAGE can be associated with decline in lung function with COPD progression, which can suggest that elevated RAGE may offer a protective advantage in COPD.

ST2 (Interleukin 1 Receptor-like 1, IL1RL1) is a member of the IL-1 receptor family. ST2 can exist in two forms, the membrane bound receptor and a soluble form, sST2. ST2 can be a prognostic biomarker for cardiac stress and can be indicative of myocardial infarction, acute coronary syndrome and heart failure. Upon stretching of the myocardium, ST2 and sST2 can be upregulated. IL-33, an IL-1 family member that promotes Th2 immunity and systemic inflammation, is the ligand for ST2. sST2 can function as a decoy for binding IL-33 and can regulate its signaling. ST2 and IL-33 can be cardioprotective and can be counter-balanced by sST2. Thus, elevated levels of sST2 can lead to elevated stress on the heart. In COPD, both IL-33 and ST2 can be elevated relative to healthy controls and may play a role in the inflammatory phenotype and thereby the pathogenesis of COPD.

Tissue Inhibitor of Metalloproteinase 1 (Timp-1) can be expressed by numerous tissues. Timp-1 is the natural inhibitor of metalloproteinases (MMPs). MMPs can function to degrade the extracellular matrix (ECM) and Timp-1 can regulate their activity. As such, Timp-1 can be important in regulating the composition of the ECM and regulating wound healing. Timp-1 can function as a growth factor. MMP-9 can be elevated in COPD and associated with tissue re-modeling. Timp-1 can be an irreversible inhibitor MMP-9. Both MMP and Timp-1 can be elevated in COPD. The ratio of MMP-9/Timp1, protease/anti-protease, can be a measure of COPD progression and severity.

Tumor necrosis factor alpha (TNF-α) is a cytokine which can be involved with systemic inflammation and considered an acute phase reactant. TNF-α can be produced by microphages and to a much lesser extent can be produced by other cells such as neutrophils, mast cells, and eosinophils. TNF-α can perform its function via two receptors, TNFR1 and TNFR2. TNFR1 can be found ubiquitously associated with all tissues, whereas TNFR2 may reside only in immune cells. TNF-α can activate three distinct pathways that seem to have opposing effects: NF-κB, MAPK pathway, and induction of death signaling. The seemingly opposing pathways can occur and function due to extensive cross-talk between the pathways. TNF-α can be significantly elevated in COPD and can be positively correlated with FEV1. Anti-TNF-α therapy can be a treatment for COPD. TNF-α can be altered during exacerbation and levels of TNF-α can lag during resolution.

Vascular endothelial growth factor (VEGF) is a member of the platelet-derived growth factors. VEGF can play a critical role in vasculogenesis and angiogenesis and primarily act on the vascular endothelium. VEGF can be highly elevated in bronchial asthma. In pulmonary emphysema, the levels of VEGF can be reduced in the pulmonary arteries. VEGF can signal through three tyrosine kinase VEGF receptors (VEGFR1, VEGFR2, VEGFR3). VEGF receptors, though alternative splicing, can exist in a transmembrane form or a soluble form. The transmembrane forms can be for signaling, while the soluble forms can function to regulate the action of the ligand (VEGF) by acting as decoys. Impaired VEGF signaling can be associated with emphysema.

Chitinase 3-like 1 (YKL-40, CHI3L1) can be secreted by numerous cell types including neutrophils, endothelial cells, macrophages and vascular smooth muscle cells. YKL-40 can be elevated and associated with inflammation and tissue remodeling. Inflammation, tissue remodeling, or the combination thereof can be a hallmark of COPD. YKL-40 can regulate anti-bacterial effects in the lung via activation of macrophages. Levels of YKL-40 can be correlated with COPD severity and can be a biomarker for decline in lung function. Levels of YK1-40 can be negatively correlated with % FEV1.

### Examples

The following examples are given for the purpose of illustrating various aspects of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Identification of a Biomarker Signature for Monitoring and Predicting Progression of Chronic Obstructive Pulmonary Disease (COPD)

Sputum was collected over multiple days post-index identification (i.e., post-hospital admission for exacerbation) and tested for a response associated with an exacerbation. The raw untreated sputum was frozen at −80 C as collected. Prior to measurement, the sputum samples were processed for measurement as follows: Frozen sputum samples were scraped out of the specimen container using a sterile metal sample paddle and weighed in a pre-weighed sterile Eppendorf tube on an analytical balance. ˜10 mg of sputum was suspended in 400 μl of the AMMP® assay buffer and vortexed vigorously for 5 minutes, followed by a 5 minute centrifugation at 4° C. The supernatant was transferred to and retained in a pre-labeled tube. The protein content of clarified sputum was quantified using a Quick Start™ Protein Quantification Kit (BioRad) and normalized to 1 mg/ml protein prior to use in AMMP® assay buffer.

The AMMP® measurement process was followed as follows: Super paramagnetic capture particles (Life Tech) were prepared with antibody specific for Platelet Factor-4 (PF4) capture. These were mixed with sputum samples that were diluted appropriately to match assay range of operation in AMMP® assay buffer. Additionally, antibody specific for RANTES (CCLS) with a hapten tag appropriate for recognition by AMMP® sensors was mixed with particles and sample to the final dilution. This facilitated sandwich type assays with intended recognition of PF4 by particle capture and RANTES by sandwich, effectively recognizing combinations of those two molecules in sample.

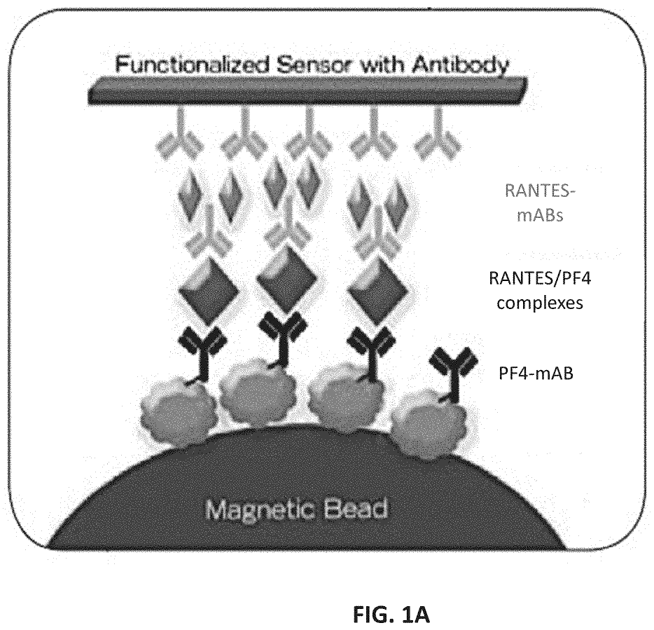

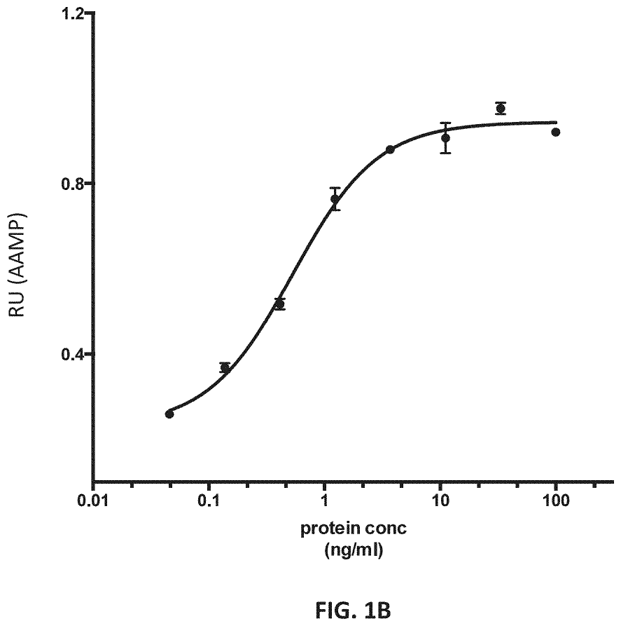

Assays proceeded in accordance with published AMMP® methods with the AMMP® sensors recognizing hapten-tagged molecules through antibody recognition and binding process. Rather than a single molecular target, in this case, a combination of PF4 and RANTES is recognized by the system. FIG. 1A demonstrates an assay design for PF4-RANTES using AMMP® technology, with a titration plot of PF4-RANTES molecular complexes shown in FIG. 1B.

In addition, sandwich assays for additional molecules as single entities (or targets) were performed as standard ELISA assays using kits from various vendors (e.g., eBiosciences).

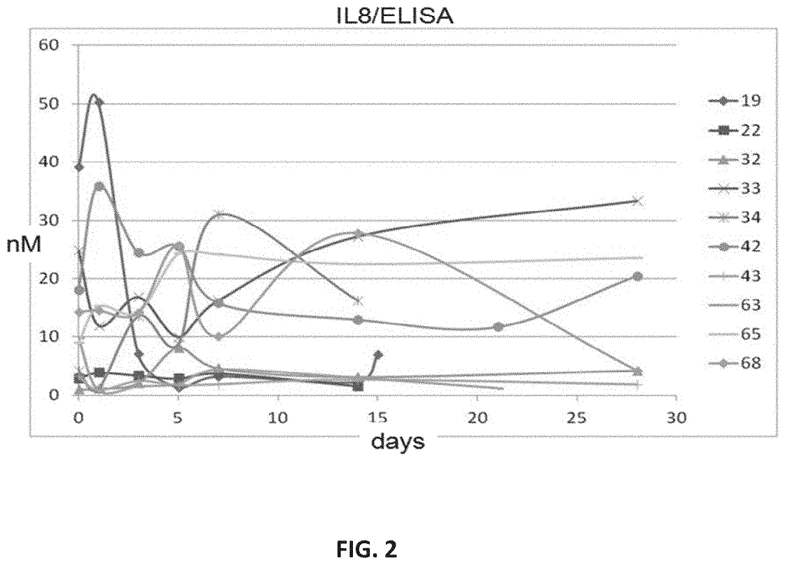

In contrast to previous reports indicating that sputum IL8, LTB4, MPO, and SLPI levels decrease with removal of bacterial infection of the lung, these markers strongly varied as a function of time. For example, FIG. 2 depicts sputum IL8 levels as measured by ELISA in an Alpha-1 Antitrypsin Deficiency (A1AD)/COPD exacerbating cohort. These results showed variable signals for all patients tested, including patients 33 and 69 who, in addition to others, are known to have recovered from the exacerbation, indicating that IL8 levels alone may not be a reliable indicator of prognosis. In addition to the limitations of using IL8 as a marker, these variable signals may reflect the limitations of sample handling (IL8 is known to rapidly degrade) or ELISA assays using sputum samples that have high protease concentrations, or that the exacerbations perhaps were not of bacterial origin.

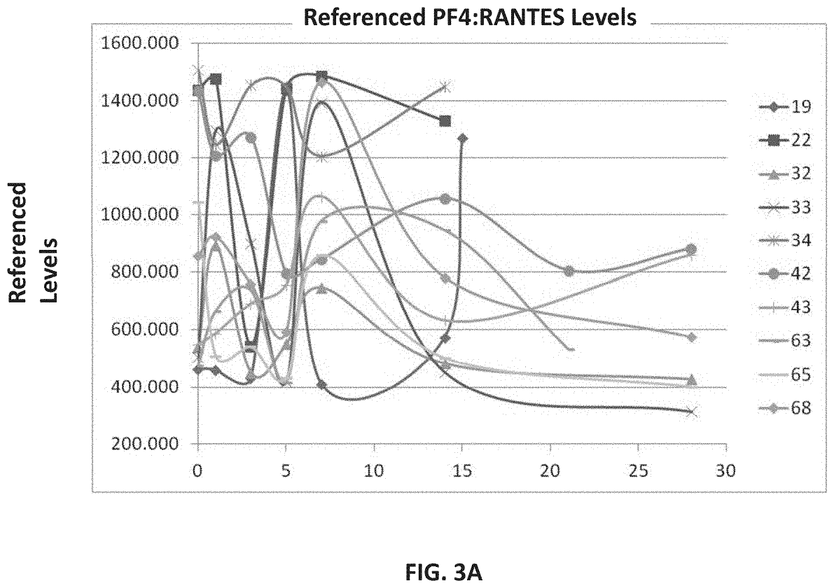

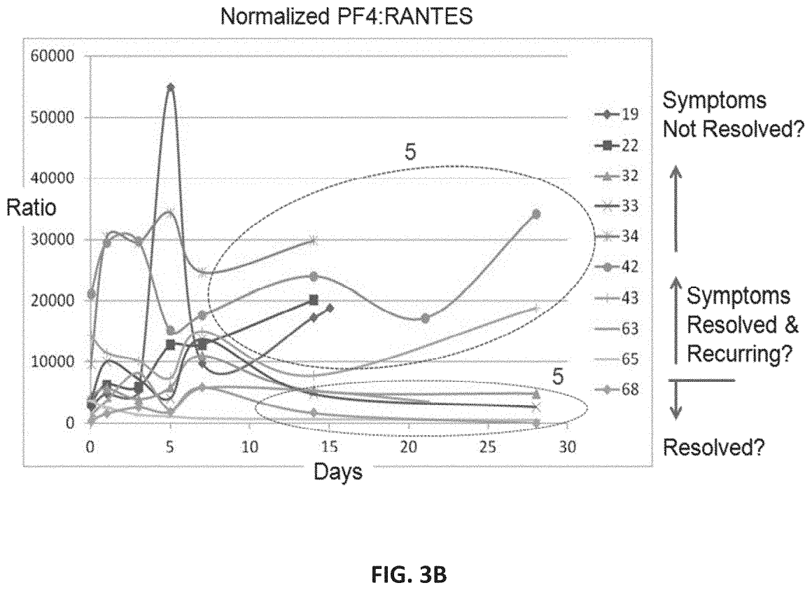

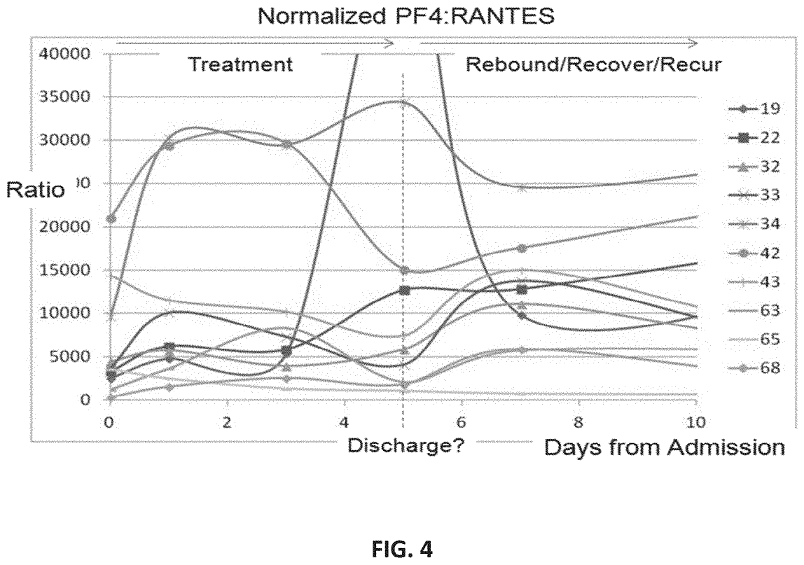

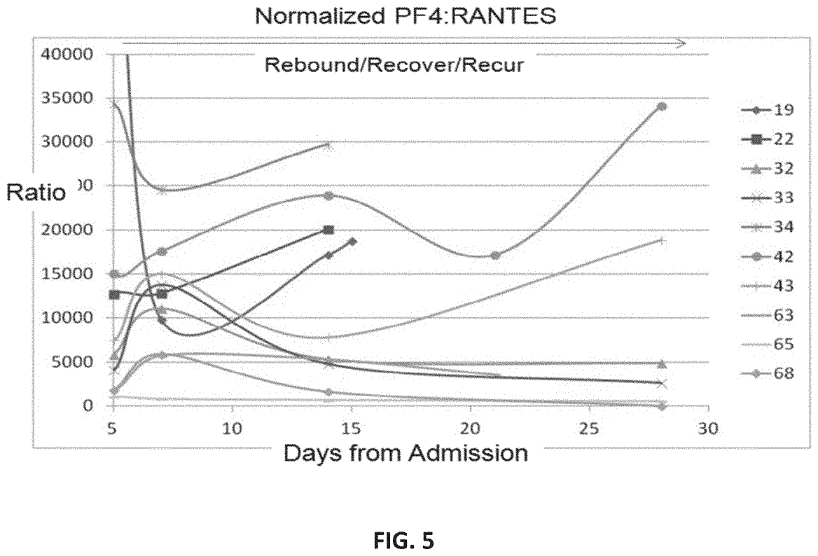

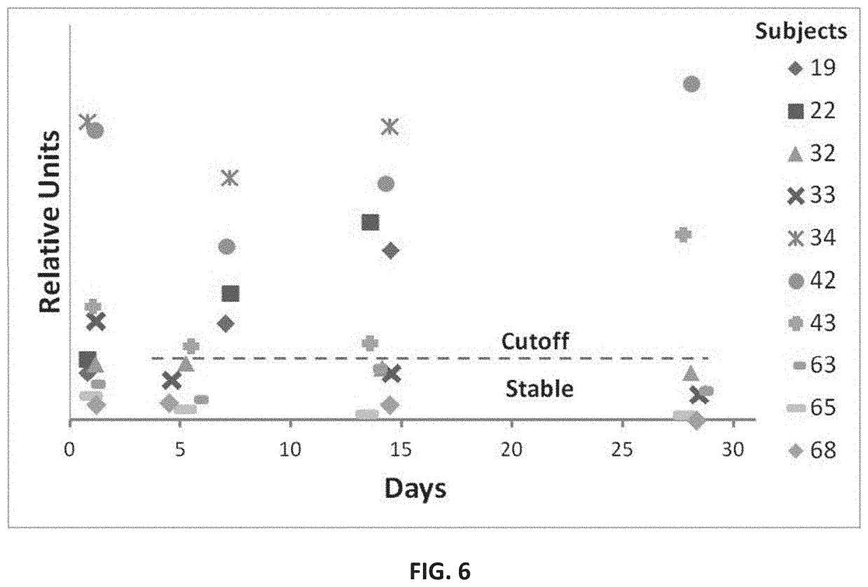

Next, the level of PF4:RANTES complexes (PRC) were measured in the sputum samples by the AMMP® method as described above. In this particular sputum data set, an additional marker, alpha-1 antitrypsin (A1AT) protein levels (eBiosciences), was employed to normalize the heteromer data at each time point (PRC ratio). Normalizing the data seemed to be important in sputum to remove the variable effects of the sample. In this case, the normalization served to further synergistically accentuate and differentiate the data trends observed compared to that of the PRC alone (see FIG. 3A versus FIG. 3B). The accentuated time trends in the data suggested that: 1) the measured heteromers may reflect important clinical outcomes (affirmed by the benchmarking knowledge that patients 33 and 68 stably resolved from their exacerbations), 2) the PRC assay may be used in the context of other protein markers, 3) importantly, diverting trends may reflect differences in inflammatory burden and clinical courses. Further, the PRC ratio data showed clearer group trends over the time course in these samples (FIGS. 3A-3B, 4, 5, and 6). Measurements from patients 33 & 68, along with 3 others, reflected recovery to stable basal states within the first 28 days (FIGS. 3A and 3B). During the 3- to 5-day period post-discharge, PRC ratio trends indicated a likely good clinical response to standard exacerbation treatment in most subjects, with a downward trend signal suggesting a decrease in local inflammation and immune response. PRC trends can discern different groups of patient responses.

These data observations are informative for several reasons: 1) Exacerbation patients have experienced a variety of treatments prior to index, or admission, such as bronchodilators, steroids of various extent and course, oxygen supplementation and mechanical ventilation. This may give rise to variable, near intractable, signals at indexing of an exacerbation event, limiting the utility of molecular biomarkers measured at this point. 2) Patients appear to receive, for the most part, normalizing treatment once they arrive. For example, 5 days post-index, a majority of the patients have reached a minimum in PRC ratio, after which they rebound upon tapering (or halting) of treatment before stabilizing in the weeks of recovery afterwards. There appears to be an advantage to measuring PRC ratio at this particular time point post-index/admission in accordance with the therapy course the patients have received. This may coincide on or near “day of discharge”, or may be day X of IV antibiotic and steroid administration, after which tapering begins based on symptoms. The resolution of symptoms in weeks after index associates 1:1 with markers measured, in this case, at day 5. 3) The data may identify patients that are likely to exacerbate prior to discharge or tapering/halting of treatment, allowing a care provider to prescribe a longer stay, a stronger or more effective course of treatment, rehabilitation or closer monitoring. For example, patients 42 and 43 may be released from the hospital because their symptoms have improved from index to day 5, however, the data demonstrates that these patients may be more likely to readmit post-discharge due to a worsening of symptoms. Thus, the data may allow a care provider to identify patients that are likely to readmit prior to discharge and to prescribe an appropriate course of action.

An additional observation is that treatment course and tapering, evident in this data between 5 and 15 days, may cause confusion in the interpretation of any molecular related test. For example, patients' molecular signatures rebound after 5 days, when strong treatments (such as steroids) are tapered. These cross with other patients still receiving strong treatments in order to control their symptoms. Thus the test limitation may be based on patient classification as it pertains to treatment course, and tied to suspected symptom resolution, but then used as a biomolecular check on underlying stability.

### Example 2. Biomarkers Measured in a First Exploratory Cohort of COPD Patients Versus Non-COPD Diagnosed Controls with and without Smoking History

In this example, molecular marker data was measured on a cohort of moderate COPD patients versus non-COPD diagnosed controls with and without smoking history (i.e., otherwise stable patients that are not exacerbating) using a combination of both AMMP® and ELISAs constructed for particular markers. The samples were plasma with anticoagulant EDTA, separated, frozen and stored at −80° C. for subsequent analysis.

In addition to molecular markers, clinical markers such as quantitative Low Attenuation Area Computed Tomography (<950 Hounsfield Units, inspiratory) (CT-LAA), lung function tests such as a ratio of Forced Expiratory Volume in 1 second to Forced Vital Capacity (FEV1/FVC) and Forced Expiratory Volume in 1 second percent predicted (FEV1% pred), that is relative to age-related loss of lung function, and diffusing capacity for the lungs for carbon monoxide (DLCO) were also known. The lung function tests were performed after bronchodilator administration, so as to assess irreversible lung obstruction. Other pulmonary afflictions such as asthma, allergy and respiratory infections may affect airway resistance and thus the need for post bronchodilator response.

Cohort demographic factors such as age and gender and smoking history were also known. As the COPD population was moderate it did not include the complexities of subjects classified as severe or very severe, and their associated treatments and co-morbidities.

This cohort did not include asthmatics by design (although there is a significant overlap of asthma with COPD, ˜10%, roughly twice the prevalence of asthma in the general population.) Notably, as an indicative control, asthma related markers did not contribute substantially to differentiation between groups, although there were some noted cases of reversible lung function in a few of the controls that had smoking history.

The cohorts included 13 COPD diagnosed subjects all of whom had smoking history, and 35 non-COPD subjects, 29 of which had smoking history and 6 with no smoking history. The biomarkers tested included: PF4, P-selectin, RANTES, CRP, MMP-9, TIMP1, MPO, IgA, IL6, Fibrinogen, Adiponectin, IgE, C1q, C3a, C5a, SAA1, and sRAGE. Data was associated for differentiation between groups, but not optimized.

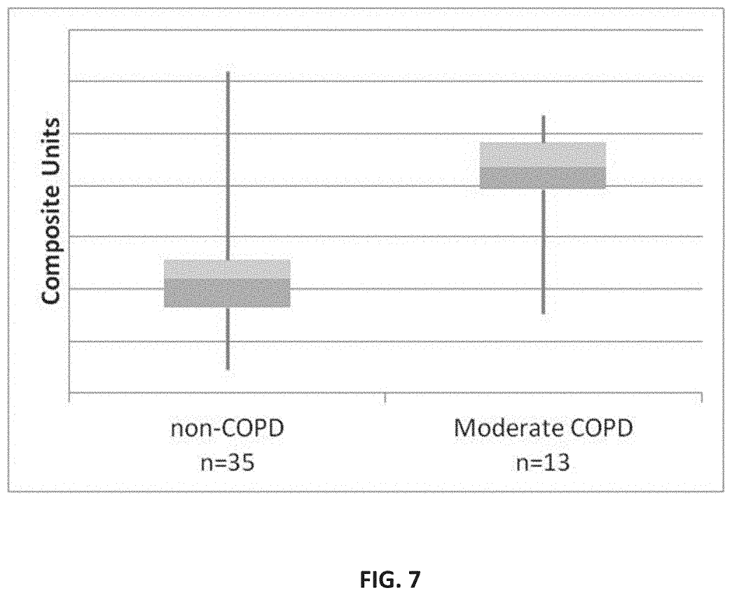

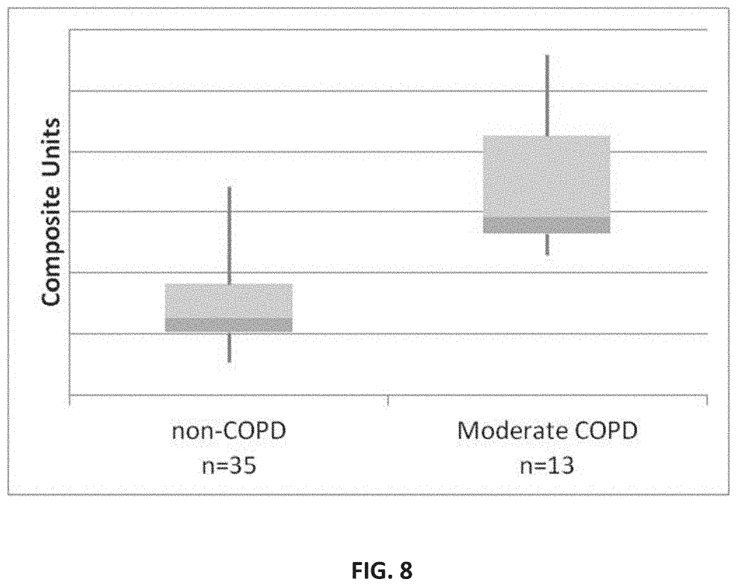

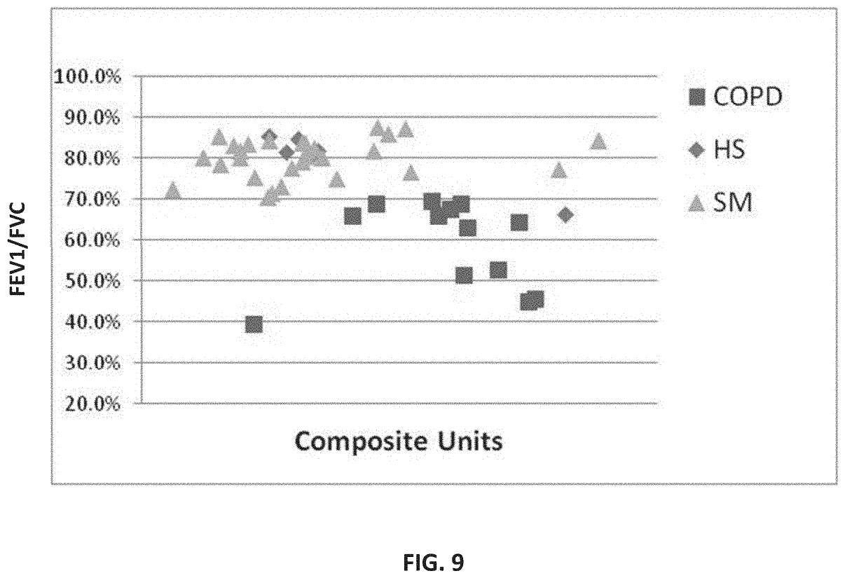

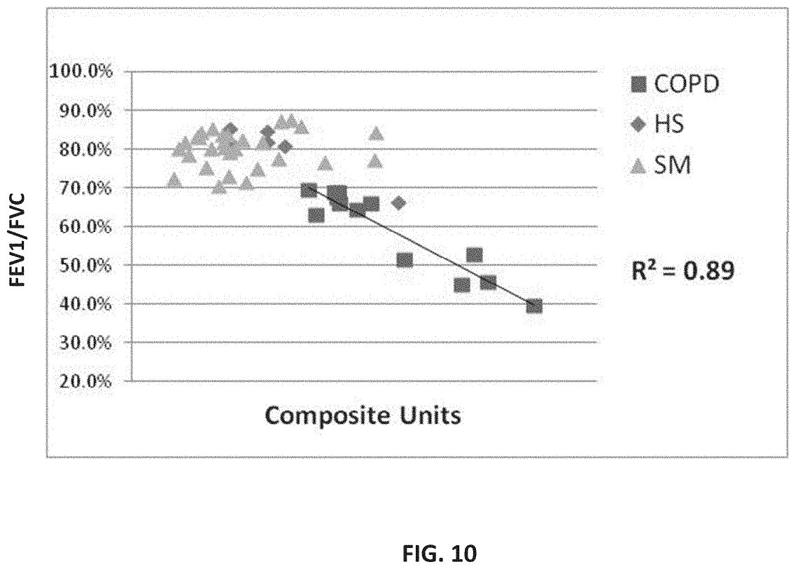

In FIGS. 7 and 8, median and interquartile ranges were plotted for a combination of molecular markers (FIG. 7) and the same molecular combination plus CT-LAA (FIG. 8). This combination of markers included CRP, MMP-9/TIMP1, IgA/TIMP1, SAA1, and PF4 multiplied by RANTES levels (PF4×RANTES). The lung function parameter FEV1/FVC was plotted for both combinations, molecular alone (FIG. 9) and molecular plus CT-LAA (FIG. 10). For reference, a post bronchodilator measured FEV1/FVC <0.7 indicates a diagnosis of obstruction and is part of the GOLD guidelines characterization of diagnosis of COPD.

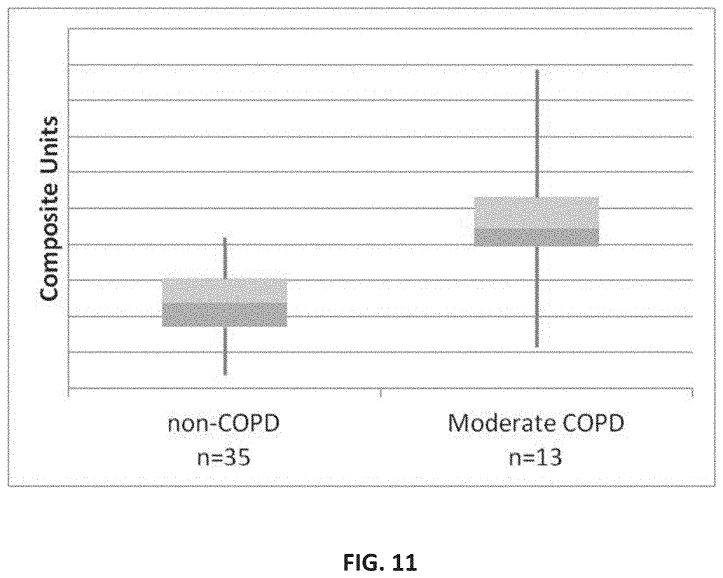

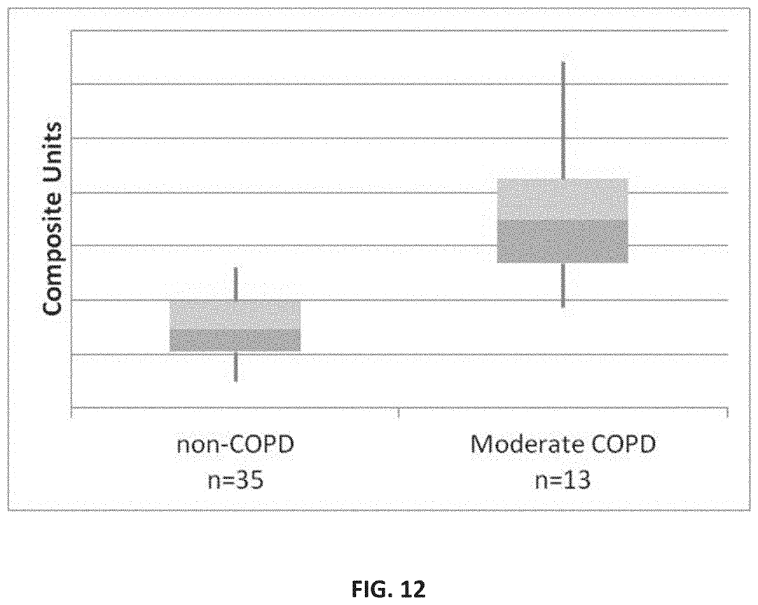

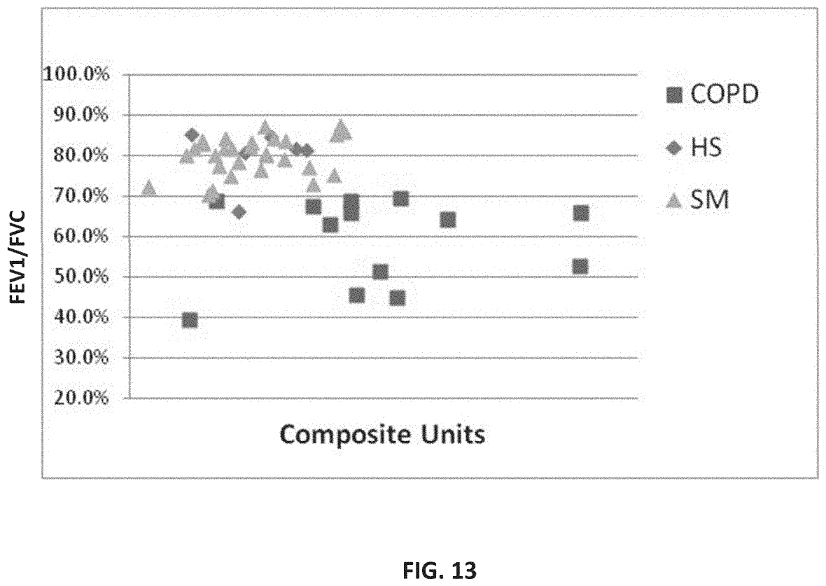

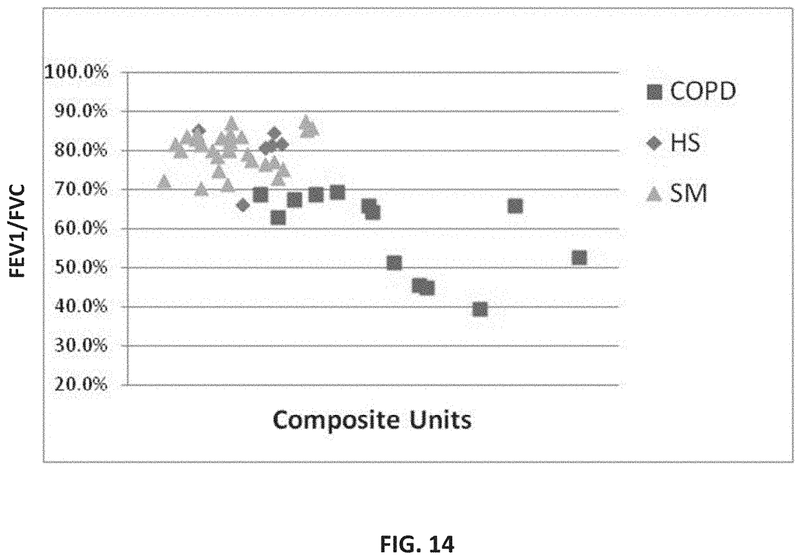

IgA, Adiponectin, and PF4 multiplied by RANTES levels (PF4×RANTES) were also examined. In FIGS. 11 and 12, median and interquartile ranges were plotted for this combination of molecular markers (FIG. 11) and the same molecular combination plus CT-LAA (FIG. 12). The lung function parameter FEV1/FVC was plotted for both combinations, molecular alone (FIG. 13) and molecular plus CT-LAA (FIG. 14).

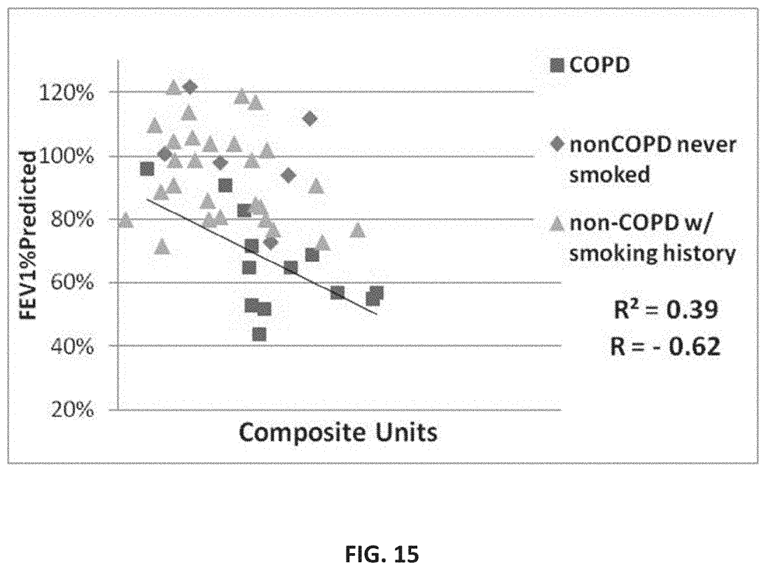

FIGS. 15-18 show a different combination of molecular markers combined with CT LAA that correlates with FEV1 percent predicted, the spirometry gauge used for severity of COPD when ratio is less than 0.7. Here, overlap with controls was present and expected. In fact many of the controls with smoking history had reduced FEV1% predicted, but with preserved ratio, FEV1/FVC >0.7, therefore not COPD by spirometry GOLD guidelines. This non-COPD group showed stronger variations in the composite index, but if the group of COPD subjects and controls with smoking history were further sub defined by risk factors, such as being an inactive smoker, then a tighter association within the COPD group resulted. By this subpopulation definition, about half of the non-COPD controls with inactive smoking history also appeared to have FEV1% pred close to that of the COPD subjects described by the composite. This important class of subjects appeared to have active COPD-like biological mechanisms and may also experience debilitating symptoms and exacerbations. Such a class may benefit from identification and treatment as they experience clinically significant events, such as exacerbations, and loss of quality of life.

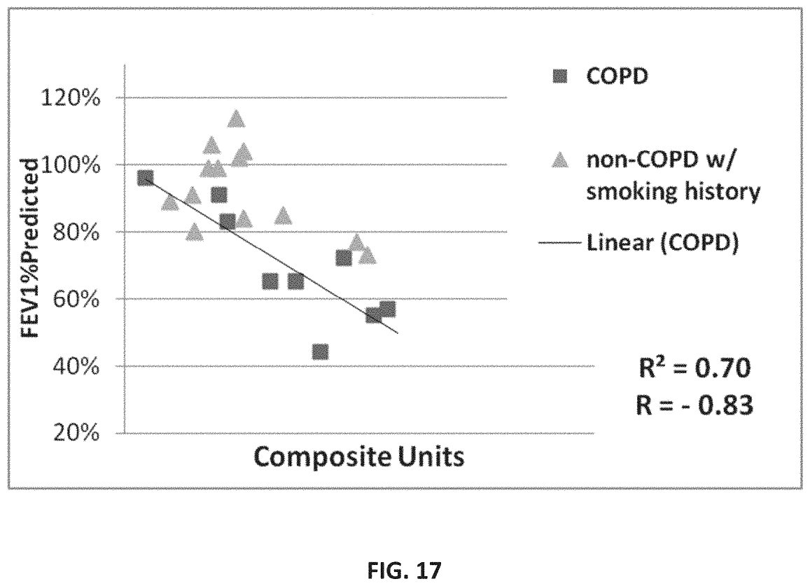

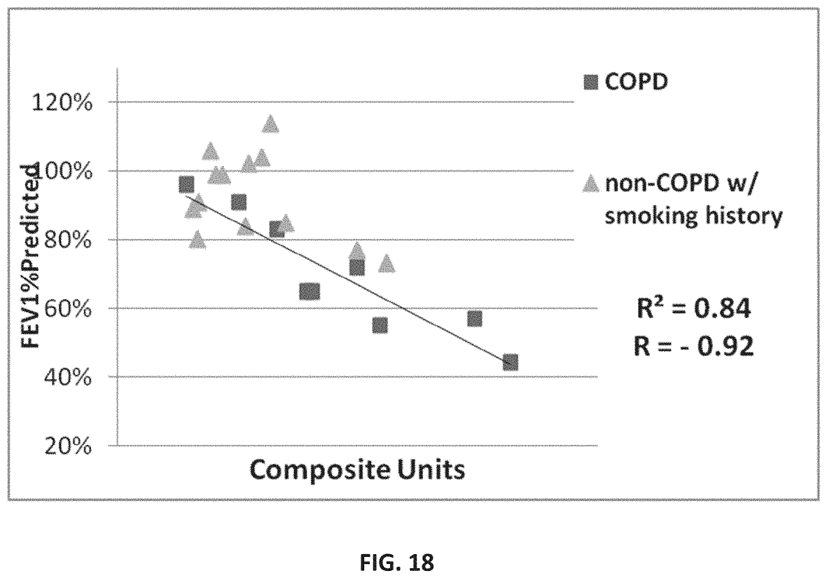

An indication in the data in FIGS. 15-18 is that subjects may be reclassified into groupings by association with correlations. For example, several subjects from the non-COPD group with a smoking history may be associated with the moderate COPD subjects shown and treated as such. The further tightening correlation in the inactive smokers shown in FIGS. 17 and 18 indicated that active smoking complicates the observation of molecular markers and that this subpopulation may be best observed independently for associating pathology. Said another way, smoking induces inflammation and adaptive immune response in reaction to foreign or non-biologically patterned material entering the lung. Some of this response is protective against degradation of the lung function, therefore not all is associated with the deleterious outcome.

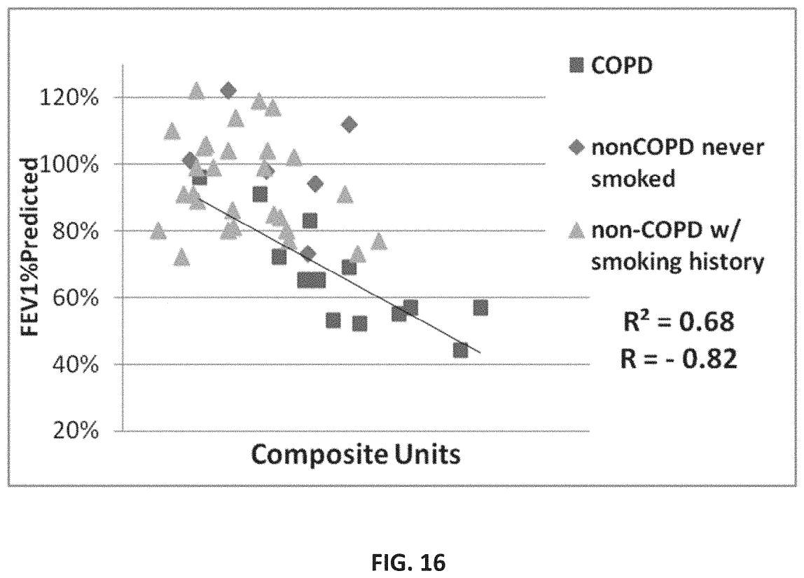

FIGS. 15 and 16 also further indicated a never smoked subject, with low lung function and high related markers (in particular CRP, MMP-9/TIMP1, and SAA1), that correlated with the COPD group. Other clinical (and molecular) variables may further distinguish this type of subject, for instance, the subject having high Body Mass Index, or a gastrointestinal and/or heart and/or liver disorder. The advantage the composite index has in identifying such a patient is being able to isolate the dysfunction related pathophysiology contributing to the clinical picture, and being able to treat as such.

Further subpopulations or categorizations, with underlying mechanisms of inflammation and adaptive immunity, may be likely. Some examples are, patients with smoking history subcategorized to active vs inactive smokers, patients with difference in blood pressure, in combination or in part, or body mass and associated indices, and/or other clinical parameters such as the 6 minute walk distance (being > or <350m), a dyspnea score (such as the modified Medical Research Council score, or modified Borg scale, or American Heart Association dyspnea scale, or Transition Dyspnea Index), patients that have never-smoked subcategorized by gender, or by allergy/asthma history, patients receiving regular inhaled or oral corticosteroids, and or synergistic steroid action drugs (e.g. theophylline), and emerging categorizations of patients by imaging, for example, patients with Expiratory Central Airway Collapse (EACS), in addition to lower airway measures such as <950 HU Low Attenuation Area as in the above data example, patients being treated for hypertension, cardiovascular disease, asthma/allergy, gastrointestinal disorder and/or diabetes, where treatments include statins, ACE inhibitors, anti-coagulants and blood thinners, dilators and steroids, protein pump inhibitors, TZD (PPARgamma) targeted therapies, and/or metformin as examples.

All subpopulations and categorization may benefit from molecular differentiation and categorization in conjunction with the more traditional clinical measures. Therapies targeted at molecular pathways, that have potential side effects, can be applied to those who need them and will benefit, resulting in efficient treatment, rather than being applied to broader indications (which they are typically trialed in).

### Example 3. Biomarkers Measured in a Second, Nine Site Clinic Based Cohort of COPD Patients and Non-COPD Diagnosed Controls

In this example, molecular marker data was measured on a large cohort (514), including all stages of COPD diagnosed patients (414) and non-COPD controls (100). Subjects had varying clinical history including COPD exacerbations reported in the past 12 months, 6 months, and 1 month. Biomarkers were measured using a combination of both AMMP® and ELISA assays constructed for particular markers. The samples were plasma with anticoagulant EDTA, separated, frozen and stored for subsequent analysis.

In addition to molecular markers, clinical markers such as lung function tests, such as a ratio of Forced Expiratory Volume in 1 second to Forced Vital Capacity (FEV1/FVC) and Forced Expiratory Volume in 1 second percent predicted (FEV1% pred), that is relative to age-related loss of lung function were also known. The lung function tests were gathered from subject medical history within the past 12 months.

Cohort demographic factors such as age and gender and smoking history were also known. This COPD population included subjects from all stages of disease spanning mild, moderate, severe and very severe, so includes the complexity of a wide variety of afflicted and their associated treatments and co-morbidities.

This cohort included asthmatics, diabetics, hypertension, obstructive sleep and those with known cardio vascular disease as well as metabolism, gastrointestinal and skeletal disorders (there is a significant overlap of these co-morbidities with COPD ranging Odds Ratios ranging 1.3-3). Notably, the controls included several of these comorbidities, as typical for an aged population. As such asthmatics and sleep disorder controls, of varying smoking status are represented.

The cohort included 414 COPD diagnosed subjects many of whom (˜95%) had smoking history. The COPD diagnosed average age was 65±8 years, 42% male, 37% active smokers, FEV1 52±22% predicted, 43% reported acute exacerbations in prior 12 months with rate 0.68/patient/year, and 13% reported being hospitalized. The 100 non-COPD subjects had approximately 40% with smoking history, and 30% with history of asthma and 40% with obstructive sleep apnea. The biomarkers tested included: Pentraxin 3, PF4, P-selectin, RANTES, PCT, CRP, Eotaxin1, HNL, MMP-9, TIMP1, IgA, IgE, IL6, Fibrinogen, Fibronectin, Adiponectin, Leptin, MCP-1, PARC, SAA1, sRAGE, and YKL-40 (CHIT3L1), Cathepsin S, Cystatin C, sST2, Resistin, C1q, Neutrophil elastase, GDF15, CC16, D-Dimer, and NT-proANP. Data was associated for differentiation between subject groupings, using biostatistical methods that rank optimized linear and log transformed selections for significance using Chi squared (p value) statistics between groupings.

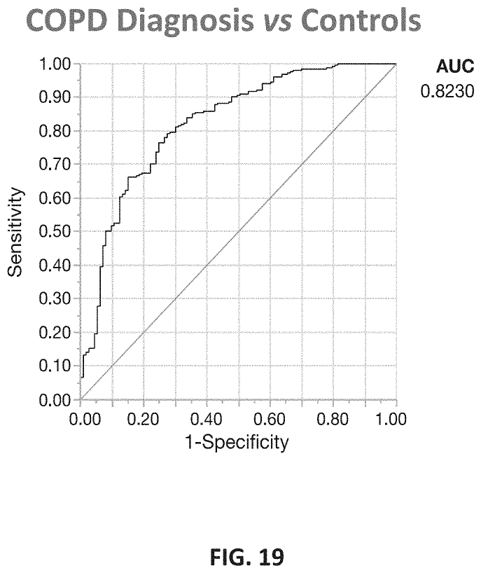

FIGS. 19-23A and 23B show different combinations of molecular markers and the ability of the statistically trained combinations to predict groups of subjects categorized by disease diagnosis and clinical measures.

FIG. 19 depicts blood biomarker combination prediction Receiver Operating Characteristic (ROC) curve for a first selection of COPD Diagnosed subjects versus non-COPD control subjects. Training of the biomarker combination was performed on approximately 268 diagnosed COPD subjects and 100 controls. The biomarker combination shown included sRAGE, TIMP-1, Leptin, Adiponectin, Fibronectin, YKL-40, IgE, Eotaxin, P-Selectin, PF4, MCP-1, CRP, SAA1, PCT, MMP-9, IgA, and HNL. Predictive performance Area Under the Receiver Operating Curve (AUROC) of 0.823 was achieved on the training set.

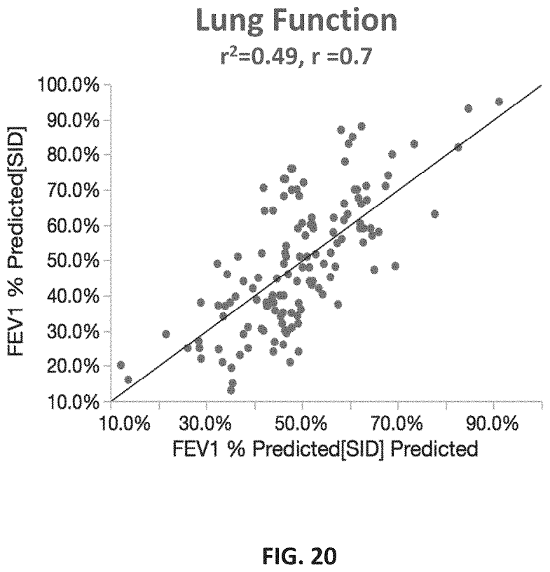

FIG. 20 depicts blood biomarker combination prediction of FEV1% predicted values.

These FEV1% predicted values were recorded in the patient medical histories in the 12 months prior to blood sample, and were reported on a continuous scale of indicating percent of the average measured for the subject age in the un-afflicted population. Included in the model shown were combinations of log transformed levels of Fibrinogen, CRP, HNL, Fibronectin, MMP-9, IgA, MCP-1, sRAGE, PCT, IgE, Adiponectin, P-selectin, Leptin, SAA1, TIMP-1. A predictive model with coefficient of determination r²=0.49, or r=0.7, was achieved in a very broad population. Additional biomarkers such as complements, complement fragments and additional endopeptidases and inhibitors are likely to improve such a model as is the clinical parameters such as those derived from imaging as demonstrated in example 2.

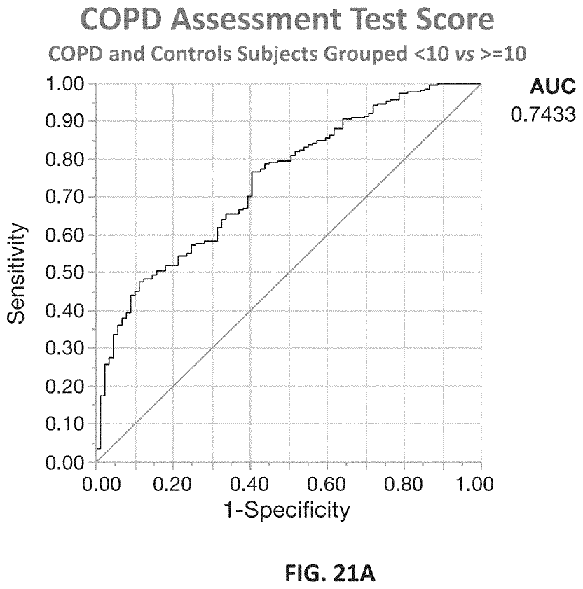

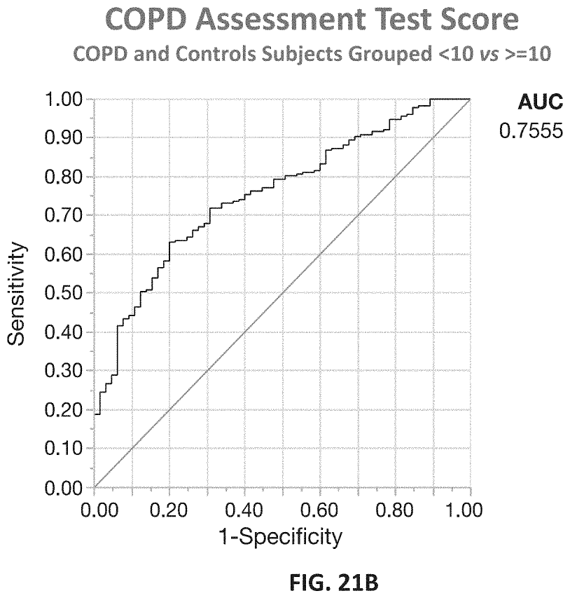

FIGS. 21A and 21B depict blood biomarker combination prediction ROC curve for COPD Assessment Test (CAT) scores. The CAT score and this categorization is recommended in the Global Obstructive Lung Disease (GOLD) guidelines for measuring increasing disease activity. While individual baseline and precision of reported CAT scores do vary due to the subjective nature of the question and response, controlled studies have shown the CAT score to dynamically increase during exacerbations and decrease to a stable level after exacerbations. FIG. 21A shows a model prediction for groups that include both COPD diagnosed and controls separated by level, <10 versus >=10 on a scale of 40. Of 368 total subjects (268 COPD Dx and 100 controls), 286 have scores >=10 while 82 have scores <10. The model trained to predict this grouping was a combination of log transformed levels of sRAGE, Eotaxin, HNL, IL6, PF4, YKL-40, SAA1, and RANTES. FIG. 21B shows a separately trained model of 293 total subjects, including both COPD and controls, 231 having scores >=10 with 62 having scores <10. This model included a combination of HNL, PF4, sRAGE, CRP, MMP-9, IgA, Eotaxin and MCP-1.

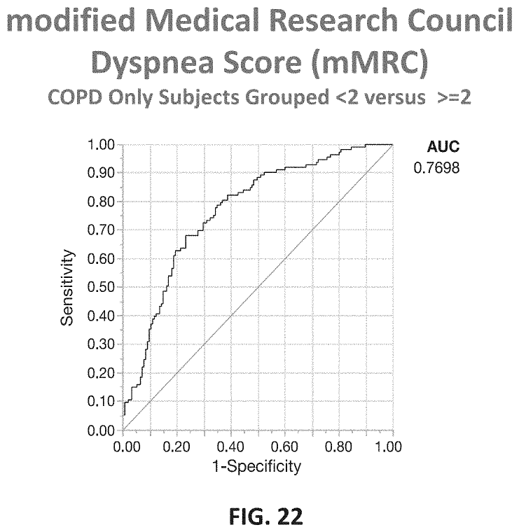

FIG. 22 depicts blood biomarker combination prediction ROC curve for modified Medical Research Council (mMRC) scores for 255 COPD diagnosed subjects. Dyspnea is a complex subjective sensation that is an important feature of respiratory disease. The MRC breathlessness scale was first published in the 1950s and has been modified since to capture a wider range of symptoms. The GOLD guidelines refer to the mMRC in categories <2 and >=2 as delineating increasing disease activity as a guide for treating COPD subjects. The combination of biomarkers depicted is Fibrinogen, PF4, Eotaxin, SAA1, YKL-40, Leptin, sRAGE, IgA, and PCT.

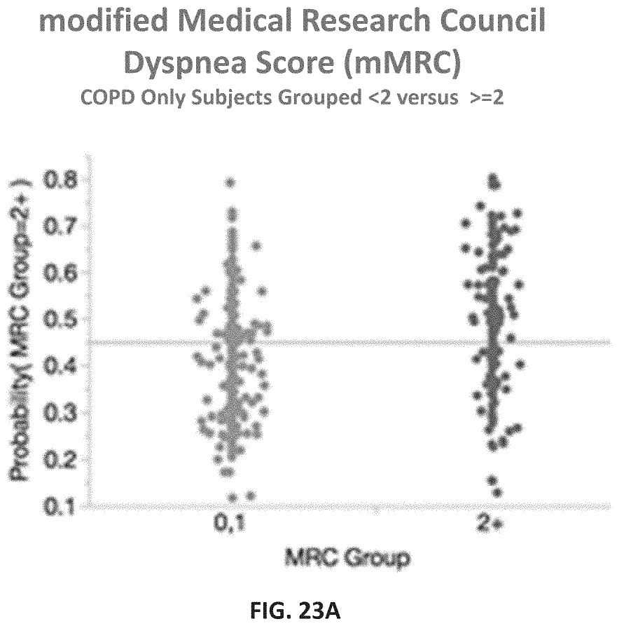

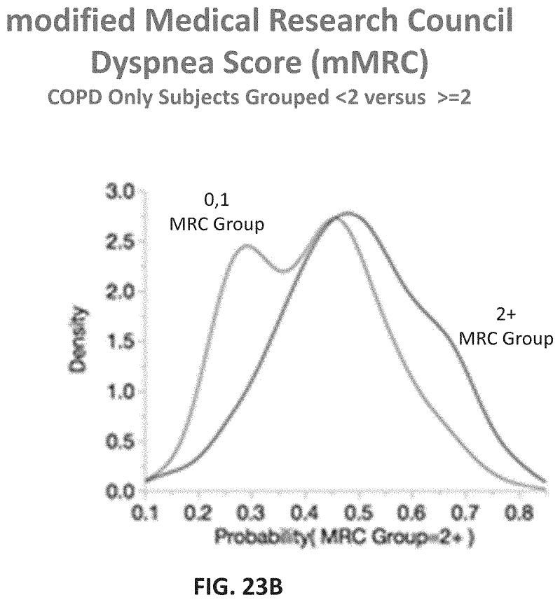

FIG. 23A and FIG. 23B depict blood biomarker combinations for 414 COPD diagnosed subjects. The combination of biomarkers giving the depicted probability versus clinical groupings of modified Medical Research Council dyspnea score, and associated probability densities per clinical grouping was Eotaxin1, PF4, sRAGE, Leptin, HNL, PARC, CRP, and MCP-1 with p-values range 0.007-0.18 and AUROC of 0.69. While the clinical grouping separation was not strong with many shared in the middle modality of probability density, each group showed uniquely separated low (<0.4) and high (>0.6) probability modes respectively. Both may have value for negative predictive value and positive predictive value for worse future outcomes. For example, chronically elevated dyspnea persistent in the presence of increasing COPD treatments have been identified in a class of COPD patients with worse outcomes.

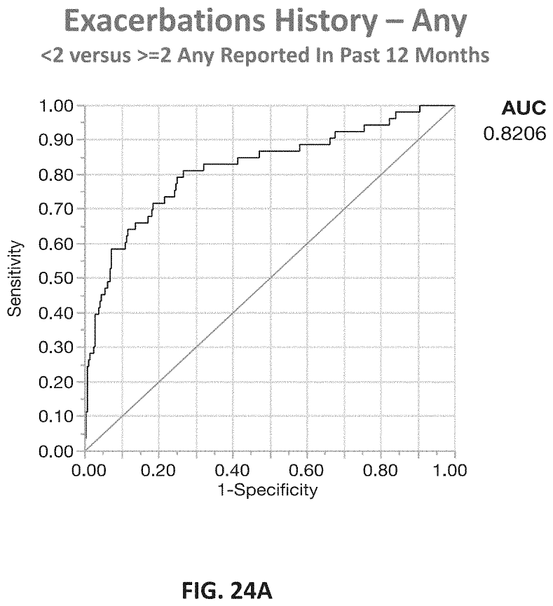

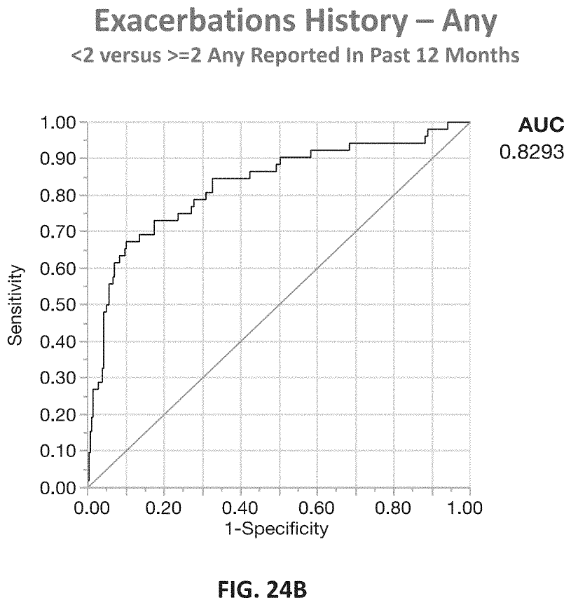

FIGS. 24A and 24B depict blood biomarker combination prediction ROC curve for COPD exacerbations history, reported in the prior 12 months. Reported exacerbations history has been shown to be one of the best indicators of risk of future exacerbation events. To date no single blood biomarker has been found to improve predictability. Four hundred and fourteen COPD diagnosed subjects were included in the analytical model training. One hundred and seventy-four of those had reported a COPD exacerbation (acute event) within the past 12 months. Sixty-one recorded two or more. Algorithms were constructed for <2 versus 2 or more reported exacerbations with and without the use of demographic and clinical variables such as gender and CAT symptoms scores.

Additionally, symptoms assessments and gender may be further used to focus the subject populations where blood biomarkers can provide some mechanistic insights as to a patients' COPD status with respect to clinical events such as exacerbations. GOLD guidelines suggest classification of patients with respect to COPD Assessment Test (CAT) scores being >=10 as having increased burden and risk. In this cohort CAT >9 gave 341 of 414, 42% male with 15% having 2 or more acute exacerbations in the prior 12 months. In a first model, markers PTX3, SAA, Eotaxin, C1q, IL6, IgE, RANTES, Leptin, HNL, Adiponectin, Cystatin C, PF4 IgA, CC16 with p values range 0.0001-0.28 combined to give AUROC 0.82 (FIG. 24A). In a second model, including CAT score as a variable, markers PTX3, SAA, IgE, HNL, IL6, Leptin, C1q, Eotaxin, TIMP-1 and CAT score combined with p values 0.0002-0.11 to have AUROC 0.83 (FIG. 24B). A third model, for females, with markers PTX3, IgE, Leptin, RANTES, NE, sST2, IL6, C1q, GDF-15, CC16, HNL, and MCP-1, had p values 0.0001-0.23 and AUROC 0.84, and fourth model, for males, with markers PTX3, Eotaxin1, Adiponectin, MMP-9, SAA, Cystatin C, Fibronectin, and CRP, had p values 0.008-0.29 and AUROC 0.86. Clearly specific biomarker combinations can complement symptoms and demographic variables to form models that better associate with patient events history (the best-known predictor of the future exacerbations events).

To evaluate predictive capability, 104 of the subjects were prospectively followed, each with 12month history of >=1 exacerbations for a mean of 100 days over winter months. Thirty-four follow up exacerbation events were recorded. An overall positive rate of exacerbations of 0.33 was observed (negative rate 0.67).

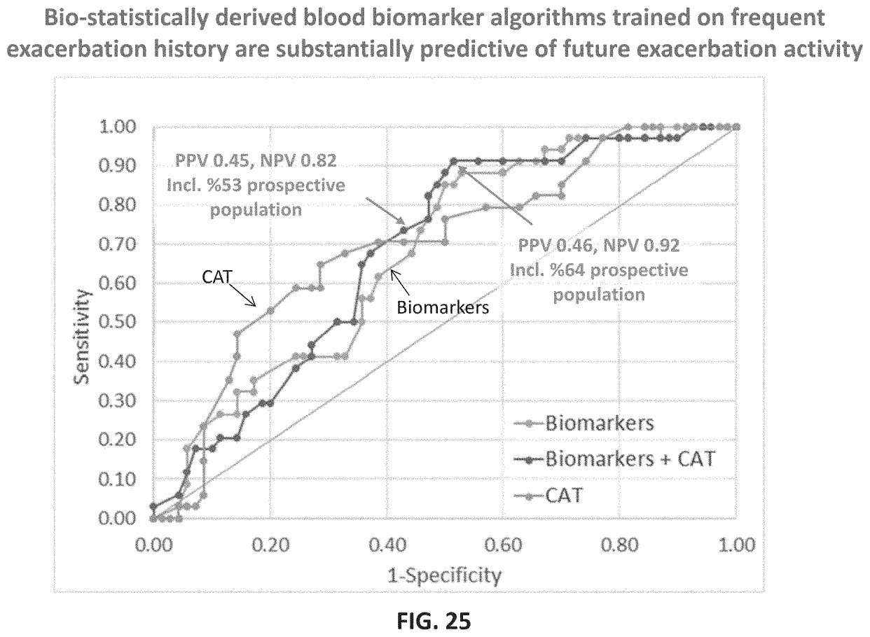

Algorithm performances, shown in FIG. 25 predicting events in the prospective collection, were AUCs of 0.68 for biomarkers only (first model listed: PTX3, SAA, Eotaxin, C1q, IL6, IgE, RANTES, Leptin, HNL, Adiponectin, Cystatin C, PF4 IgA, and CC16, additionally including sRAGE, and YKL-40), 0.68 for biomarkers+CAT score (second model listed: PTX3, SAA, IgE, HNL, IL6, Leptin, C1q, Eotaxin, and TIMP-1, additionally including YKL-40), and 0.69 for the scaled CAT score alone. All three Negative Predictive Values (NPV) models were better than that of typical clinical classifiers, which were 0.52 for >1 exacerbation history, and 0.62 for >1 exacerbation history or GOLD classifications 3&4. Prospective Positive Predictive Values (PPV) matched or were better than current clinical classifiers, 0.48 for >1 exacerbation history, and 0.38 >1 exacerbation history or GOLD stages 3&4, depending on the algorithm cut point. Algorithms utilizing biomarkers, depending on cut points selected, included a selection of mild-moderate GOLD staged subjects while deselecting low activity GOLD staged severe-very severe subjects. This indicates potential for more precise focusing of therapies to avert future events.

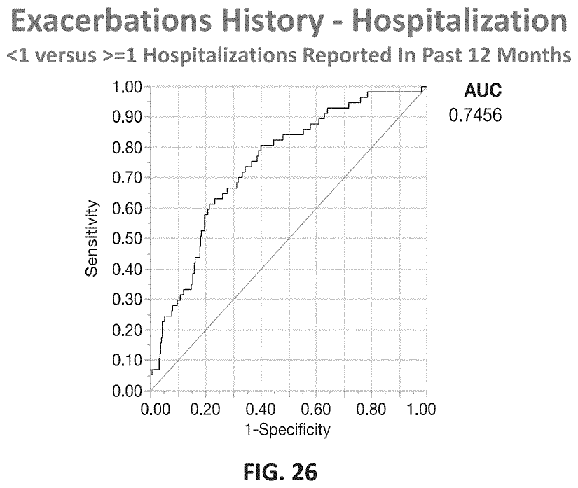

FIG. 26 depicts blood biomarker combination prediction ROC curve for COPD Exacerbations History requiring hospitalization. Hospitalizations for exacerbations within the past 12 months are an accepted indicator of increased disease activity. Of the 414 subjects, fifty-seven reported an exacerbation requiring hospitalization. An algorithm was constructed for <1 versus 1 or more reported hospitalizations. The combination of markers giving AUROC 0.75 results shown is sRAGE, SAA1, YKL-40, Eotaxin, and PF4 with p-values range 0.0001-0.065.

A prospective 12 month follow up of 138 of the 414 COPD diagnosed subjects, having breakdown by stage 1-4 of 11/48/55/24, was also analyzed. Fifty-two (38%) subjects had at least one acute exacerbation (AE) in the follow up period. Thirty-six had at least one exacerbation within 180 days of baseline. Twenty-two had at least one exacerbation within 120 days of baseline and fourteen had at least one exacerbation within 90 days of baseline. By way of comparison uni-variate, multi-variate and Random Forest associations of biomarkers with acute exacerbations (AEs) within 180 days of baseline sampling are given in Table 3. Note that clinical symptoms CAT score is of relative importance and likely would improve biomarkers models, while deemphasizing some biomarkers for choice of symptoms. Random Forest algorithms were also associated with acute exacerbations groupings (binary) and continuous time-to-next exacerbation outcomes for COPD for disease stages 1-3 only. Biomarkers are listed by importance (z score, a measure of significance in the algorithm) in minimizing predictive error of the algorithm with respect to outcomes in Table 4. The relative importance of some markers in the forest algorithm showed that non-monotonic use of biomarker levels, for example Pentraxin 3 and Cystatin C and Cathepsin is of high value in predicting propensity to having future exacerbations.

TABLE 3
Biomarkers as univariate, multivariate regression and forest algorithms
associated with acute exacerbations within 180 days after baseline sampling.
COPD Stages 1-4 Binary

| Univariate | | Multivar. Regression | | Forest Algorithm | |
|---|---|---|---|---|---|
| N = 139, 36 AEs <180 days | | N = 139, 36 AEs <180 days | | N = 139, 36 AEs <180 days | |
| Marker | p value | Biomarker | p value | Biomarker | z score |
| PARC | 0.005 | IgA | 0.011 | PARC | 9.05 |
| IgE | 0.016 | Pentraxin 3 | 0.019 | Cystatin-C | 6.90 |
| CAT Score | 0.049 | D-Dimer | 0.044 | Pentraxin-3 | 3.47 |
| IgA | 0.077 | CRP | 0.059 | Cathepsin | 3.23 |
| GDF-15 | 0.223 | RANTES | 0.078 | IgA | 3.18 |
| Fibronectin | 0.233 | P-Selectin | 0.083 | D-Dimer | 2.60 |
| Age | 0.247 | HNL | 0.144 | IgE | 2.02 |
| Leptin | 0.250 | CC16 | 0.159 | CRP | 1.94 |
| sST2 | 0.251 | Cathepsin | 0.171 | NT-ProANP | 1.44 |
| Adiponectin | 0.257 | Intercept | 0.247 | RANTES | 1.28 |
| Cystatin-C | 0.271 | Il-6 | 0.428 | IL-6 | 1.07 |
| sRAGE | 0.276 | | | sRAGE | 0.96 |
| D-Dimer | 0.281 | | | Fibronectin | 0.87 |
| CC16 | 0.289 | | | Adiponectin | 0.77 |

TABLE 4
Forest algorithm biomarker rankings for COPD stages 1-3, excluding stage 4 subjects.
Continuous time to next exacerbation and binary groupings with or without exacerbation
in future 350 days, evaluated independently in subjects with exacerbation history
(in prior 12 months) and those without.
COPD Stages 1-3

| Time to Next Event Alg. | | Binary Alg. AE = 0 prior 12 mo | | Binary Alg. AE >= 1 prior 12 mo | |
|---|---|---|---|---|---|
| N = 113, 36 AEs <350 days | | N = 62, 10 AEs <350 days | | N = 51, 26 AEs <350 days | |
| Biomarker | z score | Biomarker | z score | Biomarker | z score |
| sRAGE | 10.08 | IL6 | 10.35 | Fibronectin | 6.91 |
| CRP | 7.42 | sRAGE | 8.86 | CC16 | 3.96 |
| PARC | 4.88 | HNL | 4.19 | C1q | 3.86 |
| Cystatin-C | 3.00 | Pentraxin-3 | 3.30 | D-Dimer | 3.84 |
| Fibronectin | 2.83 | RANTES | 2.84 | sRAGE | 3.33 |
| IL6 | 1.72 | YKL-40 | 2.60 | IL6 | 2.81 |
| Pentraxin-3 | 1.14 | Resistin | 2.43 | Resistin | 2.41 |
| sST2 | 0.73 | Fibrinogen | 2.09 | PARC | 2.07 |
| IgA | 0.69 | Cystatin-C | 1.75 | Cathepsin | 1.76 |
| Cathepsin | 0.27 | CC16 | 1.21 | CRP | 1.48 |
| P-Selectin | 0.22 | CRP | 1.05 | sST2 | 1.06 |
| YKL-40 | 0.15 | Timp-1 | 0.84 | PCT | 0.68 |
| NT-ProANP | 0.01 | Neut. Elastase | 0.39 | SAA | 0.68 |
| | | Leptin | 0.26 | Timp-1 | 0.63 |
| | | | | Pentraxin-3 | 0.18 |
| | | | | Leptin | 0.05 |

Evident in Tables 3 and 4 are the varying rankings of the biomarkers with respect to exacerbations future time frames and relative history of exacerbations. sRAGE for example ranks lowly in algorithms including late stage COPD subjects. However, in many cases these patients are self-evident (and would be better typed by biomarkers rather than identified for risk). sRAGE and IL6 play a stronger role in predicting the future propensity for exacerbations in earlier stages of disease. Alternatively, PARC was stronger in algorithms including stage 4 subjects, and remains significant in forest algorithms with exacerbation history (later staged). However, PARC is less evident in those without a history of exacerbations. With this evidence it is noted that algorithms of markers vary with inclusion of exacerbations history or specific symptoms, and that stage of disease is also an important factor in marker selection and use. This will be further substantiated in the following examples.

In some cases additional disease lung function, symptoms or exacerbations history associated biomarkers may be included in the combinations, wherein the biomarkers are selected from Mannose Binding Lectin (MBL), Leptin, HNL, PTX3, sRAGE, YKL-40, PARC, IL6, C1q, A1AT, NE, Resistin, Insulin, sST2, BNP, NT-proBNP, ANP, NT-proANP, D-Dimer, Cystatin C, Cathepsin S, GDF15, CC16, total IgG, and IgG2 levels.

In some cases past diagnoses (asthma in women for example) and in some cases symptoms (wheezing in men) improve indicative risk performance over exacerbations history for future events. Given the results here for COPD symptoms scores and exacerbations history (and underlying lung function and diagnosis) it is likely a combination of these measures will improve risk assessments for acute events from either a sample taken at a single time point as shown, or from samples and scores calculated from two time points which are then compared for trend.

Further subject subpopulations or categorizations, with underlying mechanisms of inflammation and adaptive immunity, are likely. Some examples are, patients with smoking history subcategorized to active versus inactive smokers, patients with difference in blood pressure, in combination or in part, or body mass and associated indices, and/or other clinical parameters such as the 6 minute walk distance (being > or <350m), a dyspnea score (such as the modified Medical Research Council score, or modified Borg scale, or American Heart Association dyspnea scale, or Transition Dyspnea Index), patients that have never-smoked subcategorized by gender, or by allergy/asthma history, patients receiving regular inhaled or oral corticosteroids, and or synergistic steroid action drugs (e.g. theophylline), and emerging categorizations of patients by imaging, for example, patients with Expiratory Central Airway Collapse (EACS), in addition to lower airway measures such as <950 HU Low Attenuation Area as in the above data example, patients being treated for hypertension, cardiovascular disease, asthma/allergy, gastrointestinal disorder and/or diabetes, where treatments include statins, ACE inhibitors, anti-coagulants and blood thinners, dilators and steroids, protein pump inhibitors, TZD (PPARgamma) targeted therapies, and/or metformin as examples.

All subpopulations and categorization may benefit from molecular differentiation and categorization in conjunction with the more traditional clinical measures. Therapies targeted at molecular pathways, that have potential side effects, can be applied to those who need them and will benefit, resulting in efficient treatment, rather than being applied to broader population where the effects may be limited and risks or costs outweigh the potential benefits.

### Example 4. Biomarkers Measured in a Cohort of Hospitalized Exacerbating COPD Patients

In this example, molecular marker data was measured on a small cohort, of 19 subjects, sampled at admission, 7-14 days and 56 days post admission where possible. Biomarkers were measured using a combination of both AMMP® and ELISA assays constructed for particular markers. The samples were plasma with anticoagulant EDTA, separated, frozen and stored for subsequent analysis.

Cohort factors such as age and gender and COPD history are known. This COPD population included subjects spanning ages and stages of disease.

Factors surrounding the hospitalization, such as length of stay, primary treatment courses, and scheduled and unscheduled follow up during and after the 56 days are known.

The biomarkers tested included: PF4, P-selectin, RANTES, PCT, CRP, Eotaxin1, HNL, MMP-9, TIMP1, IgA, IgE, IL6, Fibrinogen, Fibronectin, Adiponectin, Leptin, MCP-1, PARC, SAA1, sRAGE, YKL-40 (CHIT3L1), sST2, cTnI. Data was associated for differentiation between subject groupings.

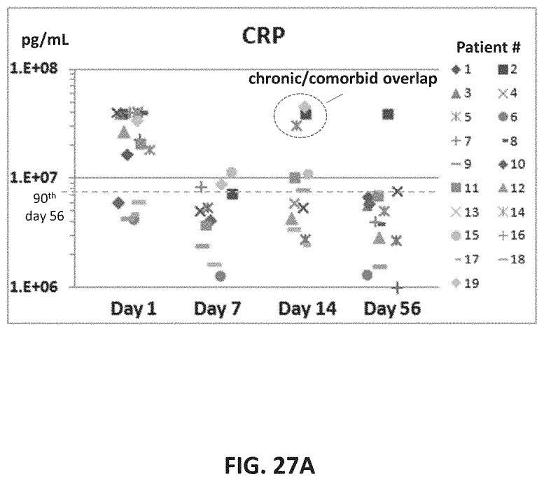

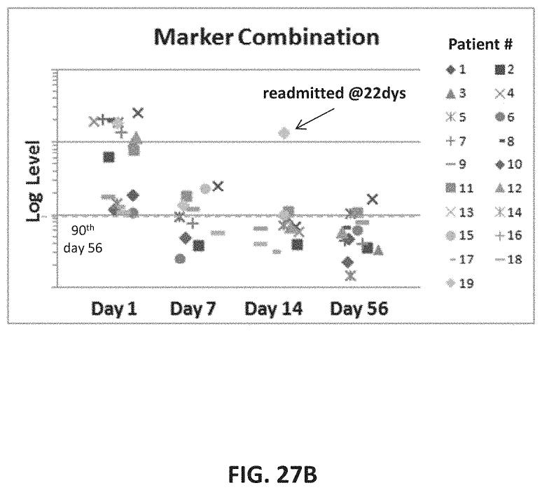

FIG. 27A depicts CRP levels versus time. Blood samples were acquired within about 1 day, 24-36 hours, of hospital admission, and where possible at about 7 days, 14 days and 8 weeks after admission, for COPD exacerbating and recovering patients.

FIG. 27B depicts combined blood biomarker levels versus time, establishing a course over time. Blood samples were acquired within about 1 day, 24-36 hours, of hospital admission, and where possible at about 7 days, 14 days and 8 weeks after admission, for COPD exacerbating and recovering patients. The combination of biomarkers shown are fibronectin, SAA1, eotaxin and sST2 (or IL1RL1). In some cases, the combination of biomarkers includes markers of acute exacerbation associated systemic or organ responses such as PCT, cardiac troponin, BNP, NT-proBNP, ANP or NT-proANP, D-Dimer, Cystatin C, Cathepsin S, and Pentraxin-3. In some cases, the combination of biomarkers includes additional immune and inflammation response molecules such as YKL-40, MCP-1, IL6, IgA, IgE and antibodies against specific infective organisms.

Ninetieth percentile of day 56 (stable) ranges for CRP and the combined biomarkers are indicated. Clear elevated levels are present at admission where a variety of entry conditions regarding presentation and time on rescue medications are indicated. Levels decreased post course of treatment, with some indications of unresolved, or yet to be cleared, effects. For nearly all patients, these cleared by day 56. One patient was readmitted to hospital during the 8 weeks post index and is indicated on the figure, having a high combined biomarker score 8-10 days prior to readmission. Notably the CRP level for this readmitted patient was also high, but so are levels for several other patients who did not readmit, as CRP can have high chronic levels. In comparison, the signal from the combined biomarkers for the persistently high CRP level patients was low in keeping with the clinical outcomes in this timeframe. Several other patients with relatively high combined biomarker scores in the day 7-14 range recorded unscheduled follow up visits within the 8 weeks of the study.

### Example 5. Measurement of Molecules that Associate and Dissociate as Complexes as a Readout of COPD Disease State

In this example, measurement in human subjects of mechanistic dysfunction, elevation or decreases in markers, particularly associated molecular complexes, may lead to effective, targeted treatment that dissociates, or results in less complexes. Generally it is well understood that patients with lung and inflammatory disease exhibit increased measures of inflammation and immune response. Example clinical markers are CRP and blood erythrocyte sedimentation rate (ESR), plasma viscosity, differential white blood cell counts (neutrophils, eosinophils, leukocytes, macrophage, etc), and a variety of additional molecular markers including IL6, IL8, TNFa, Fibrinogen, Leptin, Adiponectin, GDF-15, Mannose Binding Lectin (MBL), Pentraxin 3, Procalcitonin (PCT), MMP-9, MPO, A1AT, and Neutrophil elastase. Single associations with disease and limited multivariate combinations (e.g. one, two or three of them exhibiting changes over controls) have been researched over the years. Systemic (blood) and localized (lungs fluids, BALF, sputum etc) complexity is an accepted feature within the biological response, but to date near all molecular markers are treated as single target measurements and with single variate association to various components and measures of symptoms of disease. However, molecular complexes and transient associations may be key process contributors to the pathogenesis of disease and symptoms.

The goal of most emerging therapies is to dampen the inflammation cycle without compromising the host with adverse effects such as increased susceptibility to infection. This requires specific targeted approaches and subsequent monitoring measures to ensure that “too much” therapy does not lead to deficiency or unintended dysfunction. For example, increased inhaled steroid use, a staple of COPD treatment for exacerbations, may increase the risk of death from pneumonia.

A particular example is platelet dysfunction involving Platelet Factor 4 (CXCL4) and RANTES (CCL5). A heterodimer complex may form, and a specific inhibitor formulation, targeted at the interaction successful in inhibiting response in preclinical models. Further, the molecular complexes may be of higher order, comprising oligomers or fibrils of either molecule, and may potentially include additional complex structures including heavily glycosylated proteoglycans, which platelet factor 4 is known to associate with.

Standard inhibitors of these complexes are known, such as heparin and various synthetic forms of heparin, including sub-peptide sequences that may be constructed in either single molecular or cyclic peptide form (helping improve functionality in the presence of protease degradation). Other targeted anti-coagulants may also be successful at binding components and inhibiting complex interactions.

In a second example of complex formation in COPD, interaction complexes may form between serine proteases and serine protease inhibitors. One such interaction is that of Human Neutrophil Elastase (HNE) and Alpha-1 Antitrypsin (A1AT) molecule, where A1AT engages and neutralizes NE from elastin breakdown and associated effects. Neutrophilia is common in several inflammatory diseases and has been noted extensively in lung diseases such as cystic fibrosis, COPD, Alpha-1 deficiency. Abnormalities in this complex engagement can be measured a variety of ways; as increased end products, such as desmosine and isodesmosine, which are traditionally hard to liberate and can be non-specific in nature due to peripheral engagement of the process; or as cellular localization; or as increased cellular mechanistic (e.g. apoptosis) byproducts.

A1AT, or disease modified A1AT, is also known to complex several other factors potentially lowering its potency for lung protection. Complexes with IgE subtypes (protecting them from proteolysis in an active protease environment), IgA (with singular and polymeric forms) and with Proteinase 3 are known (PR3—evident in increase inflammatory vascular granulomytosis), but not yet investigated for importance in COPD.

Composite molecular measures that include more than two markers indicate a level of serine protease dysfunction in lung or inflammation diseases, in relevant samples acquired from patients, and this information is provided to physicians who may treat. A variety of therapy is available to treat protease dysfunction in both human and animal plasma derived and recombinant forms of biological. An example is A1AT augmentation therapy for A1 deficient patients. Another example is C1-Inh replacement therapy for patients with hereditary angioedema, with emerging application, antibody-mediated rejection and ischemic repurfusion injury.

It has been observed that CRP, Mannose Binding Lectin (MBL), and Pentraxin 3 levels are elevated in COPD but this is not found in all patients. Elevated CRP, Mannose Binding Lectin (MBL), and/or Pentraxin 3 can stimulate production of C1q and this may lead to hyper-inflammation and immune response as C1q is a major part of the classical complement pathway. Elevated C1q can accelerate cellular senescence and subsequent aging processes (engaging the Wnt pathways for example).

Alternatively, in a substantial number of smokers and COPD subjects CRP can be inconsistently low CRP and/or Mannose Binding Lectin (MBL) and/or Pentraxin 3. CRP, MBL, and Pentraxin 3 themselves degrade in the presence of active proteases, in particular the MMPs and cysteines. Furthermore, complexes of CRP, MBL, and/or Pentraxin 3 and C1q have been identified and can reduce level measurements of either, yet can still be representative of advanced inflammation and/or weakened immunity.

sRAGE is another circulating protein known to complex with other proteins in advanced inflammation. Examples of sRAGE binding proteins are Calprotectin (itself a complex of S100A8 and S100A9, otherwise known as MRP 8 and MRP 14) and HMGB1. HMGB1 can complexes with IL1beta in advanced inflammation. Low sRAGE is a significant biomarker of COPD progression and can be a marker of COPD exacerbations as well. Many of the associated proteins can be found to be elevated in COPD and associated comorbid conditions such as thyroidism (hyper-such as in Grave's condition, or hypo- such as in Hashimoto's condition) and hypertension.

Soluble ST2 can complex with IL33 during respiratory distress of the lung and cardiac distress of cardio myocytes. While increased ST2 can mediate elevated TLR type driven inflammation response, IL33 may also be locally overproduced. IL33 is a known complex of ST2, when membrane bound or soluble. IL33 can be cardioprotective and as such the elevation of ST2 with respiratory distress and corresponding complexing with IL33, if imbalanced, may lead to additional heart stress, an important comorbid condition with COPD.

In this example, a composite set of molecular markers are measured in a subject suffering from or suspected to be suffering from COPD. The molecular markers may indicate lung related, or systemic molecular complexes present in an increased inflammatory or immune system response. At least one of the molecular markers may include at least two molecules that have an association in vivo. In some cases, an increase or decrease in dysfunction is measured, and the therapy in whole or in part is adjusted or limited using this information. In some cases, the example includes measuring at least one component of the molecular complex as a single target and observing the increase or decrease in this component with the addition of a de-complexing or inhibiting reagent in vitro. The observation may include the resulting complex of the target component with the inhibitor reagent. A further example may include the incorporation of inhibiting substance in vitro and observing the dissociation related decrease in signal from the molecular complex. A further example may include the use of a measurement process using a single type of functional microparticle and a solid surface, using the AMMP acoustic assay process for example. A further example may be the composite of a molecular indication of molecular complex, components or as a whole, and a clinical marker such as quantitative CT, DLCO, output of spirometry with or without pretreatment, symptoms (CAT, mMRC, SGRQ) or condition scores or classifiers etc.

### Example 6. Measurement of Protease and/or Endopeptidase Mechanisms and Resulting Molecular Activity

This example includes the measurement of protease anti-protease mechanisms and resulting molecular activity. Such a mechanism is the action of the endopeptidases classified as Metal Matrix Proteinases, e.g. MMP-1, -3, -7, -8, -9, etc, on collagen in lung disease. As an indicator of activity, ratios of the MMP molecules to Tissue Inhibitor of MetalloProteinases, specifically MMP-9/TIMP1 level are measured. In this example, at least one molecular combination of protease and/or specifically endopeptidase levels and/or activity are measured in the composite molecular marker index for disease.

In some cases, the molecular combination includes members of the Cystatin and Cathepsin family. In some cases, the molecular combination includes proteins indicative of disease that take on protease or substrate activity such as fibronectin and/or PF4. In some cases, the molecular combination includes recognition proteins that may be degraded by increased protease activity such as CRP and members of the Pentraxin family.

In some cases, the molecular combination includes at least one member of specific families of protease inhibitors, SERPIN or TIMP family. It is also noted that these are involved in many of the mechanisms and pathways a prior mentioned: coagulation, fibrosis, fibrinolysis, complements, degradation, repair, acute phase and chronic inflammation response to recognized and unrecognized allergens or infective agents, etc. In some cases, the measurement includes at least one molecular complex indicating activity or lack of activity. Results from multiple biomarker molecular assay techniques may be used. Some assay techniques may be better than others for determining degraded and thus affinity compromised versions of the biomarkers present in sample. In some cases, the degraded biomarkers may not be desirable in the measured biomarker levels for combination and comparison, in such cases the measured effect may indicate remainder of intact molecules after disease processes are in effect. The example may include at least one result from a competition immunoassay, or competition molecular assay, to be included in a composite molecular index of disease, where at least one (or only one) type of functionalized microparticle is used, with further dependence on an interaction with a solid surface that includes an affinity reaction, that further includes association under sample mixture flows over the surface.

### Example 7. Biomarkers Measured in a Third, Predominantly Earlier Staged, Cohort of COPD Patients and Non-COPD Diagnosed Subjects with and without Smoking History, Symptoms and Respiratory Exacerbations Activity

In this example, molecular marker data was measured on a predominantly early staged cohort (a total of 341 patients, with two time points, one at baseline and one 12 months after baseline), yet including all stages of COPD diagnosed patients including non-COPD subjects with smoking history. Subjects had varying clinical history including exacerbations reported in the 12 months prior to and after the sampling time points. Biomarkers were measured using ELISA assays constructed for particular markers. The samples were plasma with anticoagulant EDTA, separated, frozen and stored for analysis.

In addition to molecular markers, clinical markers such as lung function tests, such as a ratio of Forced Expiratory Volume in 1 second to Forced Vital Capacity (FEV1/FVC) and Forced Expiratory Volume in 1 second percent predicted (FEV1% pred), that is relative to age-related loss of lung function, were also known. The lung function tests were gathered at baseline sampling.

Cohort demographic factors such as age and gender and smoking history were also known. This cohort population included subjects from all stages of COPD (post bronchodilator FEV1/FVC ratio <0.7) spanning Global Obstructive Lung Disease (GOLD) guideline categories 1 to 4. That is mild, moderate, severe and very severe categories as defined by lung function. This cohort included the complexity of a wide variety of afflicted and their associated treatments and co-morbidities. In addition, the cohort included “GOLD 0”, or unobstructed subjects with smoking history, with and without symptoms at presentation. This is important as this class of subjects, of which there are many at large in the general population, have been shown to exacerbate with similar rate to earlier staged COPD patients. Controls with no-smoking history also made up about 40 subjects in the cohort. The overall cohort included asthmatics, diabetics, hypertension, obstructive sleep and those with known cardio vascular disease as well as metabolism, gastrointestinal and skeletal disorders.

A first analysis included the 241 COPD diagnosed (GOLD stage 1-4) at both baseline and year 1. Biomarker associations were derived with respect to exacerbations activity within a year of each time point. The analysis cohort average age was 66±8 years, 62% male, 33% active smokers, FEV1 66±21% predicted, with 24% incidence of exacerbations per year that utilize health care with average rate 0.42/patient/year. Of the 241 COPD diagnosed there were 144 with significant COPD symptoms, for example by mMRC >=2 or CAT >=10 scores, had overall lower FEV1 61±18%, with increased incidence of 32% acute exacerbations per year that utilized health care with average rate 0.57/patient/year.

The blood biomarkers tested in this cohort included: Pentraxin 3, PF4, P-selectin, RANTES, PCT, CRP, Eotaxin1, HNL, MMP-9, TIMP1, IgA, IgE, IL6, Fibrinogen, Fibronectin, Adiponectin, Leptin, MCP-1, PARC, SAA1, sRAGE, and YKL-40 (CHIT3L1), Cathepsin S, Cystatin C, sST2, Resistin, C1q, Neutrophil elastase, GDF15, CC16, D-Dimer, and NT-proANP.

Data was associated for differentiation between subject groupings related to exacerbations in the 12 months prior to or after sampling, using biostatistical methods that rank optimized linear and logarithm transformed selections for significance using Chi squared (p value) statistics between groupings.

Subject groupings for frequent acute exacerbations (AE), that is >=2 in the time period with health care utilization treated with either steroids or antibiotics, with respect to history and/or future, were as in Table 5. Biomarkers sRAGE, PARC, Leptin, RANTES, IgA, C1q, IL-6 were found to separate these groupings significantly. Additional markers Resistin, Cystatin C, IgE, PF4, MMP-9, TIMP1, CRP, sST2, and NT-proANP were less significant but showed differentiating effects within subject groupings which may be of benefit in application to larger numbers of subjects and groupings.

TABLE 5
Frequent Acute Exacerbations Outcomes Summary

| AE | | | | |
|---|---|---|---|---|
| historic | AE future | Group | | |
| count | count | notation | N (%) | |
| ≤1 | ≤1 | 0, 1 0, 1 | 416 | (86%) |
| ≤1 | >1 | 0, 1 2+ | 27 | (5.6%) |
| >1 | ≤1 | 2+ 0, 1 | 28 | (5.8%) |
| >1 | >1 | 2+ 2+ | 11 | 2.2%) |

Subject groupings for having any acute exacerbations (AE), that is >=1 in the time period with health care utilization treated with either steroids or antibiotics, with respect to history and/or future, were as in Table 6. Biomarkers sRAGE, IL-6, Leptin, HNL, Adiponectin and quantitative CT measure related to small airway disease, were found to separate these groupings significantly. Biomarkers sRAGE, Leptin, NT-proANP, Pentraxin 3, HNL, adiponectin and a quantitative CT measure related to small airway disease, were found to separate these groupings significantly. Additionally, IL6, IgA, MMP-9, TIMP1, fibrinogen, P-selectin, RANTES, Cystatin C, YKL-40 and

PARC showed some effects separating groups which may provide additive value in differentiating larger groups of subjects.

TABLE 6
Any Acute Exacerbations Outcomes Summary

| AE | | | | |
|---|---|---|---|---|
| historic | AE future | Group | | |
| count | count | notation | N (%) | |
| 0 | 0 | 0, 0 | 305 | (63%) |
| 0 | 1+ | 0, 1+ | 69 | (14%) |
| 1+ | 0 | 1+, 0 | 62 | (13%) |
| 1+ | 1+ | 1+, 1+ | 46 | (9%) |

Multi-variate models were trained for any (1 or more events prior or after the sample time point) versus no (0) activity. Both time points were combined as subjects substantially change state between sampling dates. A first mode combined biomarkers HNL, CC16, Pentraxin 3, sRAGE, sST2, NT proANP, Leptin, IgE, IgA, Eotaxin, P-selectin, had AUC 0.73 with p-values ranging 0.0001-0.29. Additional models included CRP, MMP-9, GDF 15, YKL-40 with significance. A model that included CAT score and Sex as variables with markers HNL, sRAGE, Pentraxin 3, sST2 and YKL-40 had AUC of 0.76 for activity with all p values <0.01. CAT score alone achieved an AUC of 0.68 predicting activity in this group.

A second analysis included GOLD 0 (with smoking history yet unobstructed by ratio test), GOLD 1's and 2's with FEV₁>=65% predicted. This sub selection had N=202 with average age was 63±9 years, 62% male, 39% active smokers, FEV₁ 87±13% predicted, with incidence of 15% acute exacerbations per year that utilize health care with average rate 0.27/patient/year. A second sub selection cohort was formed by further restricting by age to <=67 years giving N=116 where now average age was 58±7 years, 63% male, 51% active smokers, FEV₁ 87±14% predicted, with average of 20% acute exacerbations per year that utilize health care with average rate 0.39/patient/year. A third sub selection only included those from the first sub selection that were symptomatic by COPD Assessment Test (CAT) >=10 at baseline. This third sub selection cohort for analysis comprised N=134 subjects with average age was 63±9 years, 54% male, 49% active smokers, FEV₁ 78±17% predicted, with average of 21% acute exacerbations per year that utilize health care with average rate 0.44/patient/year.

By way of biomarkers data and exacerbations outcomes both baseline and year 1 were concatenated, to make 2×N for the various sub selections analyses. The exacerbations were aggregated within a year of each time point for grouping analysis, those having any exacerbations versus those with none.

An associative model for exacerbation activity in the first cohort sub selection included HNL, IgE, Leptin and subject age, with p values range <0.001-0.02, and has an AUROC of 0.73. An associative model for exacerbation activity in the second cohort sub selection (<=67 years) included HNL, Leptin, IgE, YKL-40, P-selectin, IgA, TIMP-1, SAA1, IL-6, and subject age (not insignificant), with p values range 0.003-0.35, and has an AUROC of 0.76 (95^{th}CI 69-83).

In the third sub selected cohort, including those with CAT>=10, CAT scored at baseline predict exacerbations activity with AUC of 0.66 (95^{th}CI 59-73). Associative models of biomarkers only included biomarkers HNL, PCT, PF4, IgE, IL-6, Eotaxin1, SAA1, PARC, TIMP-1, IgA, sRAGE, which can reduce to a model with only IgE, PCT, PF4, HNL, Eotaxin1, PARC, IL-6, with AUROC of 0.70 (95^{th}CI 64-73). A biomarkers model for males included IL-6, MMP-9, IgA, PCT, IgE, HNL, PARC had AUROC of 0.80 with p-values range 0.025-0.24. A biomarkers model for females included MMP-9, SAA1, PF4, HNL, sRAGE, TIMP-1, CRP, YKL-40 and has AUROC of 0.79 with p-values 0.002-0.09. These combined to give AUROC of 0.80 (95^{th}CI 71-85). An alternative model (with extended biomarkers) for males included Pentraxin 3, NT-ProANP, HNL, CC16, YKL-40, Cathepsin.S, MMP-9, Fibrinogen with AUCROC of 0.82 and p values ranging from 0.0005-0.09. An alternative model (with extended biomarkers) for females included sRAGE, Eotaxin, PF4, sST2, HNL, YKL.40, MCP-1, IL6, CRP, Pentraxin 3, CC16, MMP-9, Adiponectin, IgA, NT-ProANP, PCT with AUROC 0.87 and p values ranging from 0.0004-0.17. A combination of 10 biomarkers with COPD associated symptoms includes HNL, PF4, PCT, IgE, SAA1, sRAGE, PARC, IL-6, IgA, TIMP-1, and CAT score, with range of p-values 0.01-0.28, and has AUROC of 0.77 (95^{th} CI 71-85).

A third analysis included GOLD 0 (with smoking history yet unobstructed by ratio test), GOLD l's and 2's with FEV₁>=50% predicted. This sub selection had N=255 with average age was 65±9 years, 61% male, 38% active smokers, FEV₁ 80±17% predicted, with average of 16% acute exacerbations per year that utilize health care with average rate 0.28/patient/year. Exacerbations were aggregated within 2 years of the year time point. The aggregation weighted future exacerbations higher than historical, and exacerbations within 12 months of year 1 time point higher than 12-24 months from the time point. Random Forest algorithms were trained to the exacerbations outcomes for the aggregated cases and biomarkers ranked z-score (a relative measure of importance in minimizing algorithm prediction error) tabulated in Table 7 for three forest algorithms: all subjects, female only and male only. AUCs for the algorithms were in the range of 0.65-0.70. Of note is the differing importance (z score, a measure of reducing error) of biomarkers within each model, notably Fibronectin and sRAGE are markers with <1 z score in the male algorithm but high contribution to minimizing error in the female algorithm, and in opposing fashion Leptin and PF4 are high contributors in the male algorithm but substantively lower in the female algorithm. These observable effects indicate the need for algorithms that incorporate biomarkers levels non-monotonically and potentially with demographic and clinical variables included to further raise specificity.

TABLE 7
Random forest biomarker algorithm biomarker
contributions for GOLD 0-2 stage of disease.

| ALL | | Female | | Male | |
|---|---|---|---|---|---|
| Marker | z-score | Marker | z-score | Marker | z-score |
| Leptin | 15.1 | Fibronectin | 13.0 | Leptin | 7.5 |
| sRAGE | 5.7 | sRAGE | 11.6 | PF4 | 5.9 |
| IgA | 3.8 | IgE | 6.2 | HNL | 5.9 |
| PF4 | 3.8 | IgA | 5.2 | RANTES | 5.8 |
| TIMP1 | 3.6 | Eotaxin | 5.0 | IgA | 5.1 |
| CRP | 3.6 | P. selectin | 4.9 | MMP9 | 5.0 |
| IL6 | 3.5 | Leptin | 3.0 | PCT | 4.0 |
| P. selectin | 3.5 | RANTES | 2.1 | TIMP1 | 3.2 |
| RANTES | 3.0 | IL6 | 1.4 | IL6 | 2.0 |
| Fibronectin | 2.7 | YKL.40 | 0.8 | C1q | 1.8 |
| HNL | 2.6 | PF4 | 0.4 | YKL.40 | 1.3 |
| Eotaxin | 2.4 | C1q | 0.0 | CRP | 1.2 |
| YKL.40 | 2.2 | | | IgE | 1.1 |
| MMP9 | 2.0 | | | Adiponectin | 1.0 |
| C1q | 1.6 | | | | |

### Example 8. Biomarkers Measured in a Fourth, High Hospitalization Rate Cohort of COPD Patients

In this example, molecular marker data was measured for a high hospitalization rate cohort of COPD patients. Frequent severe acute exacerbations pose high risk to COPD patients, requiring complex care and engagement to help mitigate them. Veterans are at particularly high risk with three times the rate of overall disease compared to the general population and exhibit increased biological complexity with higher rates of risk factors and comorbidities. The subjects of this cohort had varying clinical history. Biomarkers were measured using ELISA assays constructed for particular markers. The samples were plasma with anticoagulant EDTA, separated, frozen and stored for analysis.

In addition to molecular markers, clinical markers such as lung function tests, such as a ratio of Forced Expiratory Volume in 1 second to Forced Vital Capacity (FEV1/FVC) and Forced Expiratory Volume in 1 second percent predicted (FEV1% pred), that is relative to age-related loss of lung function, were also known. The lung function tests were gathered at baseline sampling.

Cohort demographic factors such as age, gender and smoking history were also known. This cohort population included subjects predominantly from GOLD stages 2 to 4 (i.e., moderate, severe and very severe categories as defined by lung function). This cohort included the complexity of a wide variety of afflicted and their associated treatments and co-morbidities. The overall cohort included asthmatics, diabetics, hypertension, obstructive sleep and those with known cardio vascular disease as well as metabolism, gastrointestinal and skeletal disorders.

This cohort comprised of 113 male veterans with complete history. The analysis cohort average age was 69±6 years, 100% male, 22% active smokers, FEV1 47±18% predicted, CAT scores 17.2±9, with 50% having acute exacerbations, 36% hospitalized in the prior 12 months (rates 1.25 and 0.76/patient/year respectively), 22% with frequent severe exacerbations >1 Emergency Department visit or >1 hospitalizations.

The blood biomarkers tested in this cohort included: PF4, P-selectin, RANTES, PCT, CRP, Eotaxin1, HNL, MMP-9, TIMP1, IgA, IgE, IL6, C1q, Fibronectin, Adiponectin, Leptin, MCP-1, PARC, SAA1, sRAGE, YKL-40 (CHIT3L1), Cathepsin S, Cystatin C, Resistin, Neutrophil Elastase, sST2, D-Dimer and A1AT.

Nine univariate biomarkers, sRAGE, Eotaxin1, C1q, HNL, IgE, A1AT, TIMP-1, MMP-9, D-Dimer, were found with p values ranging 0.007-0.17 for associations with frequent severe acute exacerbations defined as >1 Emergency Department visit or >1 hospitalizations in the prior 12 months. Prior any acute exacerbation history, CAT scores and steroids each were significant, p values <0.03, while FEV1, smoking, age, and Charlson comorbidity score were not.

All biomarkers were used to build ensembles of blood biomarker classification trees (random forests). Thirteen blood biomarkers had Forest based z scores >2 (eq. p values <0.05), C1q, sRAGE, Resistin, Cathepsin S, IgE, PF4, YKL-40, A1AT, Neutrophil Elastase, HNL, P-selectin, Eotaxin, and MCP-1. Slightly different models were found depending on threshold for risk, for example one including RANTES but excluding MCP-1 was found if favoring positive predictive value over negative predictive value for the historical outcomes.

When included, CAT score had the highest z score (lowest p value <0.001). Algorithms with and without CAT score achieved significantly improved positive predictive value, or negative predictive value, compared to CAT (essentially only) at all thresholds that were studied (Table 8).

TABLE 8
Forest algorithms including blood biomarkers substantially predict frequent severe
exacerbations history

| | | | | | | | 95% CI for |
|---|---|---|---|---|---|---|---|
| Threshold | Algorithm | Sn | Sp | NPV | PPV | AUROC* | AUROC* |
| 0.4 | Essentially CAT only | 0.31 | 0.85 | 0.80 | 0.38 | 0.62 | (0.49, 0.74) |
| — | 13 Biomarkers | 0.35 | 0.89 | 0.82 | 0.47 | 0.72 | (0.61, 0.83) |
| — | CAT + 13 Biomarkers | 0.35 | 0.90 | 0.82 | 0.50 | 0.78 | (0.68, 0.88) |
| 0.3 | Essentially CAT only | 0.38 | 0.76 | 0.80 | 0.32 | 0.62 | (0.49, 0.74) |
| — | 13 Biomarkers | 0.58 | 0.74 | 0.85 | 0.39 | 0.73 | (0.62, 0.84) |
| — | CAT + 13 Biomarkers | 0.62 | 0.76 | 0.87 | 0.43 | 0.79 | (0.69, 0.89) |
| 0.2 | Essentially CAT only | 0.62 | 0.60 | 0.84 | 0.31 | 0.63 | (0.51, 0.75) |
| — | 13 Biomarkers | 0.85 | 0.55 | 0.92 | 0.36 | 0.73 | (0.62, 0.84) |
| — | CAT + 13 Biomarkers | 0.85 | 0.57 | 0.93 | 0.37 | 0.78 | (0.68, 0.88) |

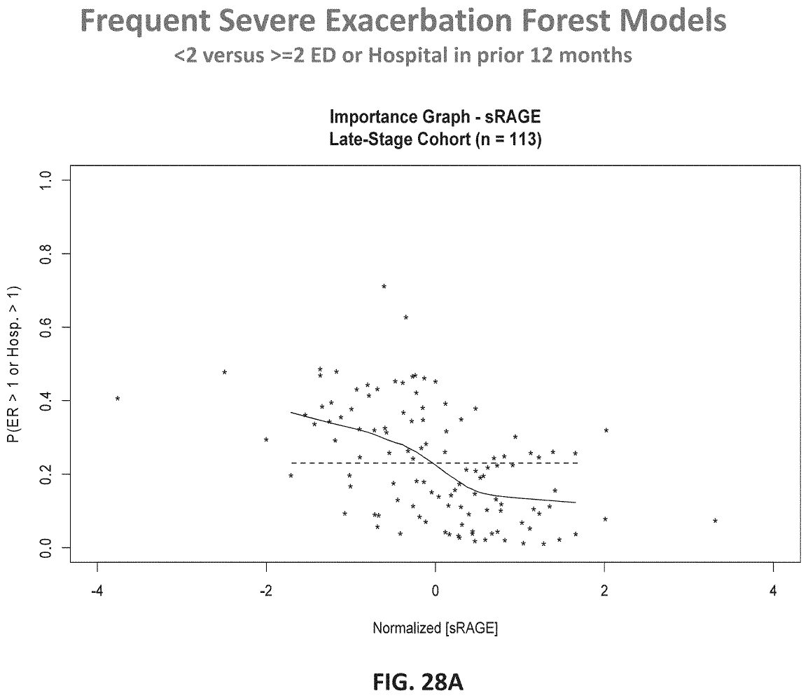

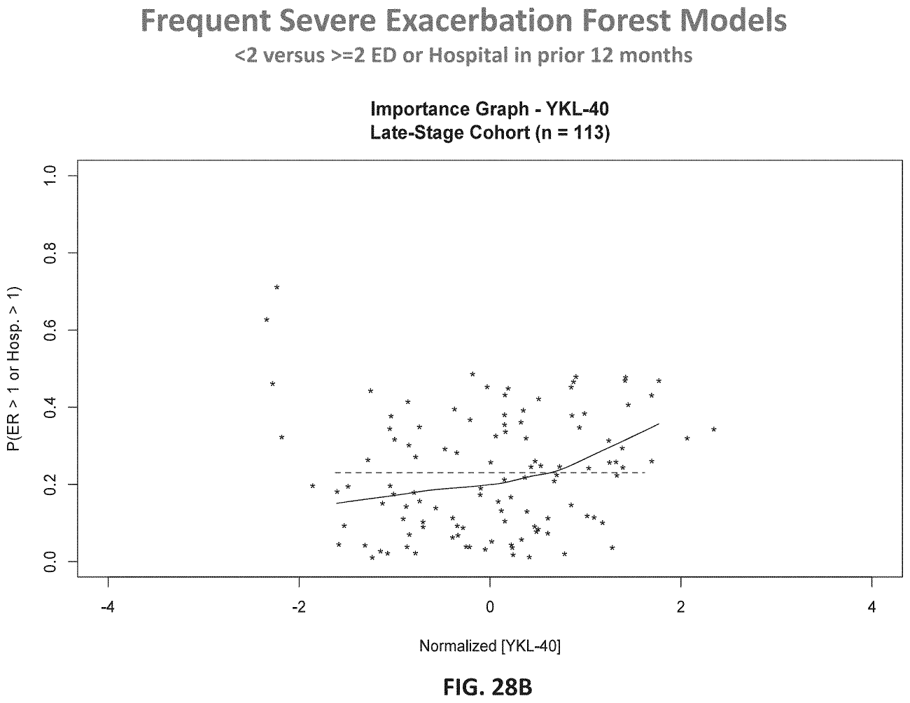

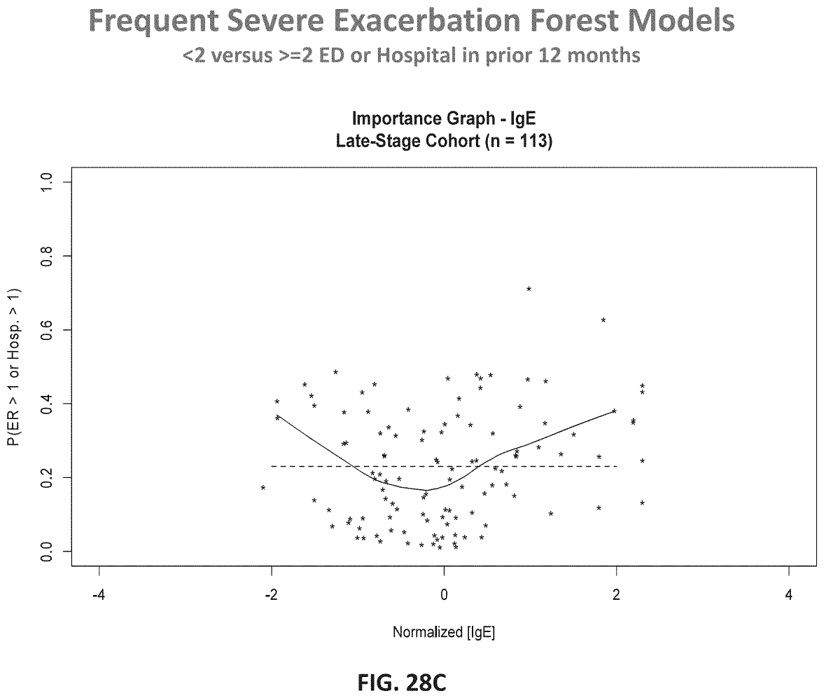

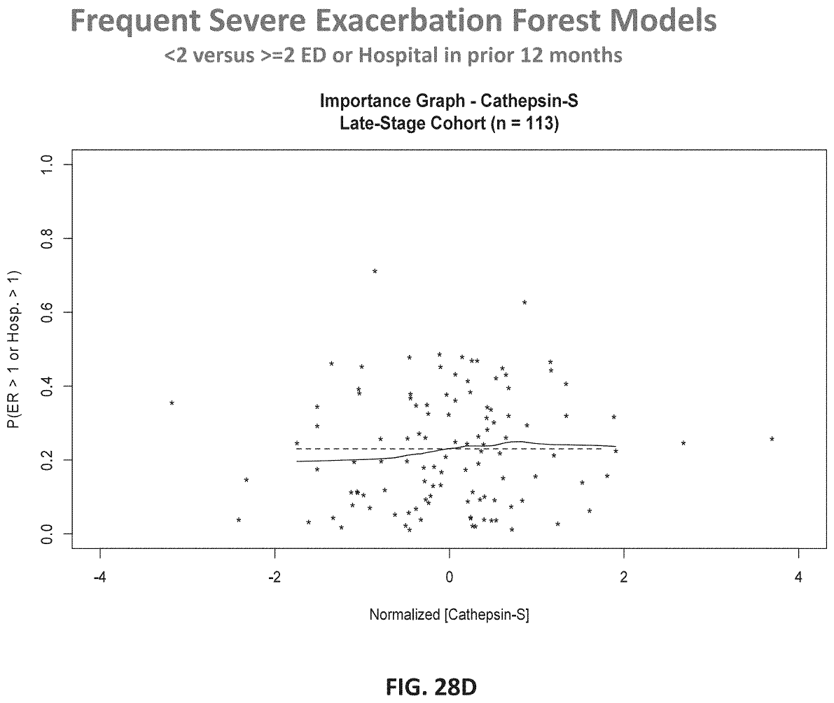

FIGS. 28A-28D show example marker levels versus forest algorithm predictions for sRAGE, YKL-40, IgE and Cathepsin S. This forest prediction included symptoms CAT score (not shown). Forest algorithms allowed for multiple levels of each included marker to be used in deriving the (they comprised a forest of 1000s of decision trees, where decision levels were found at each set that minimizes prediction error to the outcomes. The overall outcome rate, for frequent severe exacerbation in this group was shown as a dashed line. Solid lines for each marker showed trends with marker level for the predicted outcome. Lower (sRAGE), higher (YKL-40 although note low has value too), both lower and higher (IgE) and distributed (in the case of cathepsin) were observed indicating that multiple non-monotonic levels of multiple biomarkers play a role in advanced disease.

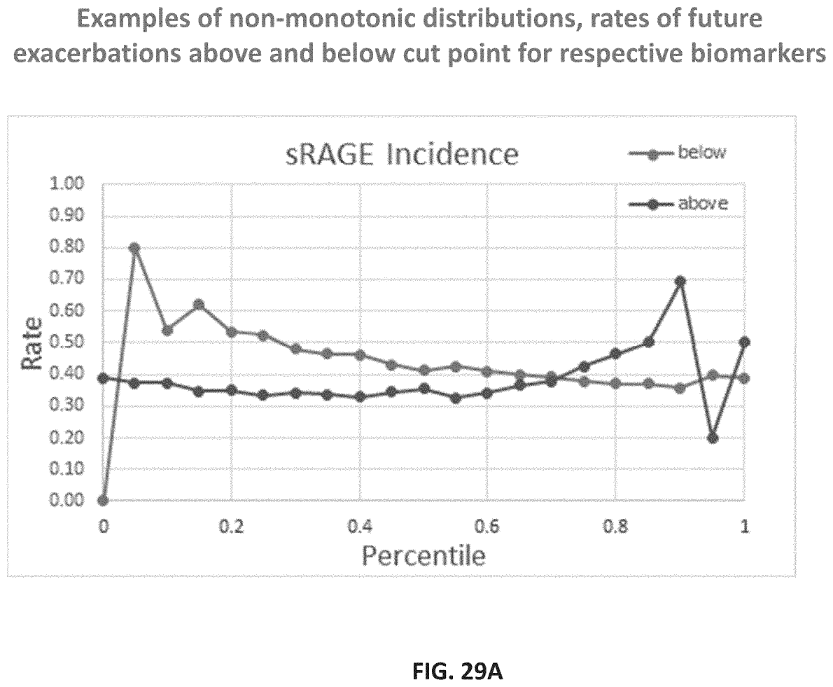

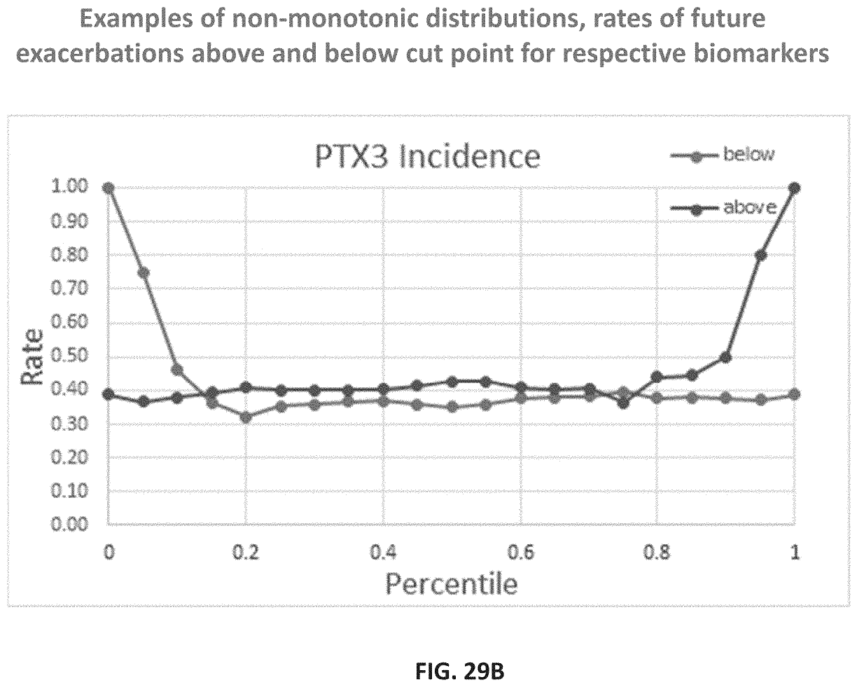

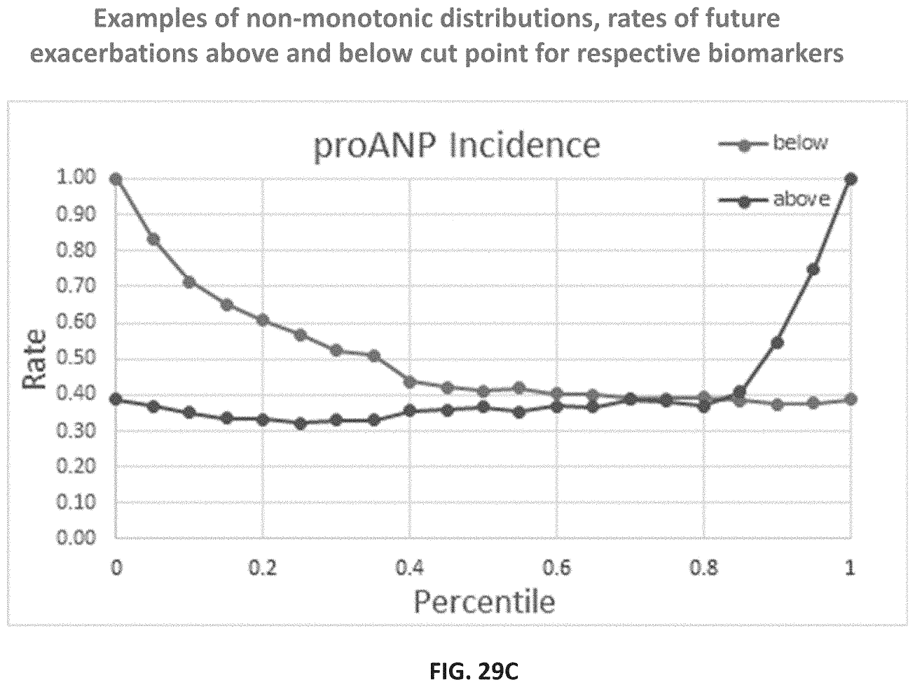

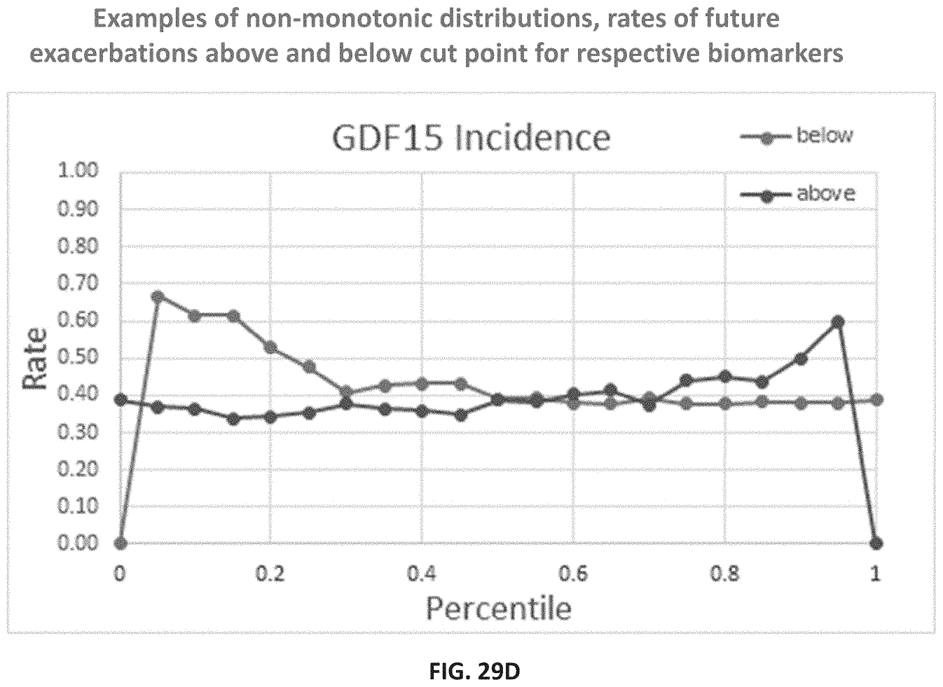

FIGS. 29A-29D show incidence rates for COPD exacerbations as a function of percentiles cut off values for four representative biomarkers that have significance for events: sRAGE, Pentraxin 3, proANP and GDF 15. Separate curves were given for above and below percentile cutoffs where evidently rates rise with respect to entire range rates in both low range and high ranges of biomarker values. The shown data were established analyzing the prospective 12 month follow up of 138 of the 414 COPD diagnosed subjects referenced in example 3. Theses 138 subjects with COPD diagnosis had breakdown by stage 1-4 of 11/48/55/24. Fifty-two (38%) subjects had at least one acute exacerbation (AE) in the 12 months follow up period analyzed.

### Example 9. Identification of Rising Risk Populations

Identifying rising risk populations of patients can have utility for implementation of disease management programs as a generalized intervention to provide better care for these patients. Disease management programs can result in improved outcomes and burden on the patients and care organizations. These patient populations may be early staged, including, symptomatic people with risk factors such as obesity and/or smoking that are likely in process towards significantly obstructed airflow, or, they may be focused on those with diagnosed clinically significant disease, in terms of airway obstruction and symptoms, yet with low care utilization history. Applicable disease management programs may include closer patient interaction, medications use, compliance and adherence, and increased monitoring by respiratory health care professionals, elements of pulmonary rehabilitation, and/or exercise and wellness related engagement and encouragement programs, tailored elements of telemedicine through digital interactive interfaces that record and transmit episodic or daily symptoms, vital signs, and key measures of pulmonary function, such as peak flow or oxygen saturation, to a centralized disease management system. The disease management system may analytically process the individual data for semi-automated observation and intervention by health care professional trained in patient clinical management procedures specialized for identified rising risk patients. Management procedures may also include applicable additional lines of therapy or therapies targeted at reducing the risk for future severe or progressively severe events. A therapy aimed at reducing the risk for future severe or progressively severe events can include advanced combination inhalation formulations, such as long acting bronchodilator and anti-muscarinic, and potentially also steroid in a so-called triple therapy in a single inhaler device, or the provision of some or all of the three as single devices in an open format for incorporation in a treatment plan. Additionally, dual action therapies can be used. An example of a dual action therapy can be the provision of bronchodilation action as well as an anti-inflammatory therapy, such as those listed in Table 1.

While exacerbations frequency and symptoms can assess disease activity and associated additional lines of pharmacological treatment, they are, depending on the stage, often not persistent measures to guide inclusion in disease management approaches. Indeed, a substantive fraction of future exacerbations and severe events can come from the groups of patients with mid-range symptoms and relatively infrequent prior events, making them challenging to identify ahead of time for better engaged and managed care.

Over longer time scales (year to year or years) the BODE score may be used to gage an individual COPD patient's disease progression. The BODE score can be a measure combined of individual elements: B (body mass index), 0 (obstruction as defined by FEV1% predicted groups), D (dyspnea as defined by the mMRC) and E (exercise capacity as defined by the six-minute walk test). Quantiles of the BODE score can associate with, and can be predictive of increased mortality risk, but the score can be cumbersome to assess, having to perform interventional spirometry and the variable six-minute walk tests in periods of relatively stable disease, and not encompassing of earlier staged patients that present with increasing dynamic risk of increasingly severe and progressive events.

A stratification blood test indicating increased risk for patients presenting with clinically significant disease with respect to increasingly severe exacerbations was developed. Along with recent history of exacerbations (past 12 months) and symptoms (CAT and mMRC) and state scores such as BODE, and Hospital Anxiety and Depression Score (HADS), this test algorithm may incorporate lung function parameters such as FEV1, medications use such as inhaled steroids, smoking status active, inactive or never, and additional clinical factors such as age and sex.

A first analysis of biomarker combination for rising risk included 75 COPD diagnosed patients with mid to late staged COPD (substantially GOLD 2-3, or moderate and severe disease). Biomarkers tested were Pentraxin 3, PF4, P-selectin, RANTES, PCT, CRP, Eotaxin1, HNL, MMP-9, TIMP1, IgA, IgE, IL6, Fibrinogen, Fibronectin, Adiponectin, Leptin, MCP-1, PARC, SAA1, sRAGE, and YKL-40 (CHIT3L1), Cathepsin S, Cystatin C, sST2, Resistin, C1q, Neutrophil elastase, GDF15, CC16, D-Dimer, and NT-proANP. The biomarkers were measured and evaluated at two-time points spaced 12 months apart, so longitudinal biomarkers but the analysis groupings remained static as they were established on baseline clinical information. Exacerbations incidence rate was 0.39 for moderate to severe exacerbations and 0.21 for severe only. Biomarker associations were derived with respect to two groups of patients, a rising risk group that had approximately twice the rate of severe exacerbations compared to a lower risk group.

Univariate biomarkers significance for the rising risk grouping and ensembles of blood biomarker classification trees (random forests) for the rising risk grouping are given in Table 9. Note that, compared to univariate analysis biomarkers IgE, IgA, Leptin, HNL and GDF 15 had higher relative importance in the ensemble models reflecting that ensembles of decision trees allows for multi-variates and multi-level groupings of biomarkers to be utilized in the algorithms trained on response variables (in this case rising risk grouping). The ensemble algorithms gave effective AUCs of 0.75 (0.67-83 95^{th} CI) and 0.80 (0.73, 0.87 95^{th} CI) for the groupings with ˜ predictive values, negative and positive, of >0.7.

Several of the top markers in the algorithms had non-monotonic relationship to overall exacerbation rate, and thus ensemble (or randomized machine learned) algorithms were better suited that log-monotonic models for outcomes. This is especially reconciled with the growing comorbid conditions that existed within the rising risk populations.

TABLE 9
Univariate and Forest derived biomarkers and relative significance/importance
for rising risk of severe events groupings of patients in a first cohort.

| Uni-var | | | | Forest Biomarkers | |
|---|---|---|---|---|---|
| Biomarkers | p-value | Forest Biomarkers | z-score | w/age and sex | z-score |
| Age | 0.0001 | IgE | 15.27 | Age | 22.89 |
| NT-ProANP | 0.0013 | NT-ProANP | 14.70 | IgE | 13.35 |
| Pentraxin-3 | 0.0178 | Pentraxin-3 | 13.32 | NT-ProANP | 12.85 |
| Neutrophil Elastase | 0.0192 | Leptin | 6.37 | Pentraxin-3 | 11.29 |
| MCP-1 | 0.0375 | Neutrophil Elastase | 6.21 | IgA | 7.20 |
| PF4 | 0.0593 | IgA | 5.74 | GDF-15 | 6.08 |
| CC16 | 0.1047 | MCP-1 | 5.51 | Leptin | 5.86 |
| D-Dimer | 0.1287 | D-Dimer | 4.97 | HNL | 5.73 |
| IgE | 0.1450 | HNL | 4.93 | Neutrophil Elastase | 4.79 |
| HNL | 0.1674 | PF4 | 4.88 | SAA | 4.59 |
| Leptin | 0.1812 | IL-6 | 4.36 | D-Dimer | 4.13 |
| IL-6 | 0.1978 | PARC | 4.34 | PF4 | 3.80 |
| IgA | 0.2201 | SAA | 3.97 | IL-6 | 3.04 |
| sRAGE | 0.2393 | RANTES | 3.81 | MCP-1 | 3.01 |
| SAA | 0.3407 | GDF-15 | 3.09 | CC16 | 2.82 |
| RANTES | 0.3682 | sRAGE | 2.72 | PARC | 2.70 |
| PARC | 0.4065 | Cathepsin | 2.61 | Cathepsin | 2.67 |
| PCT | 0.4086 | YKL-40 | 2.36 | sST2 | 2.49 |
| C1q | 0.4782 | sST2 | 2.26 | Sex | 2.45 |
| Fibronectin | 0.4993 | TIMP-1 | 2.19 | RANTES | 2.27 |
| Others | >0.5 | Eotaxin | 1.94 | P-Selectin | 2.04 |
| | | CC16 | 1.28 | sRAGE | 1.38 |
| | | Fibronectin | 1.05 | Cystatin-C | 1.28 |
| | | C1q | 0.63 | Fibronectin | 0.61 |
| | | | | CRP | 0.61 |
| | | Cystatin-C | 0.34 | YKL-40 | 0.48 |
| | | | | MMP-9 | 0.45 |
| | | | | TIMP-1 | 0.33 |

### Example 10. Generation of a Disease Activity Algorithm for Calculating a Disease Score Comprising Specific Biomarker Selections from Clusters of Blood Biomarkers that Associated with Disease Activity of COPD, Including Exacerbations, Symptoms, Lung Function and Structure (CT Measures) Across Elucidated Examples, Disease Stages, Indicative of Past Disease Activity Control, and Reflective of Post Sampling Date Risk of Future Events and the Relative Severity of the Events

Combined evaluation of the examples given in this specification, where biomarkers and groups of biomarkers both together and independently have association during, after and prior to acute exacerbations of COPD, with early, mid or late stages of disease, have resulted in the following non-obvious systematic analysis: A disease activity algorithm for calculating a disease score comprising blood biomarkers and associated score for a patient suffering from COPD, or similar small airways related disease(s), that indicates whether a subject's COPD is controlled, or they are relatively uncontrolled, or whether they are prone to near or further term acute events, and/or whether they may benefit from, or have benefited from increased or decreased therapy and pharmacological treatment of the disease and disease aspects, including comorbid couplings, is formulated from at least four biomarkers selected with the following procedure:
Selection of at least one biomarker measurement with specificity for sRAGE, PF4, P-selectin, RANTES, TIMP1, PARC, CC16, NT-proANP, or Fibrinogen;
Selection of at least one biomarker measurement with specificity for CRP, Pentraxin 3, Adiponectin, D-DIMER, IL6, MCP-1, Cathepsin S, or Cystatin C;
Selection of at least one biomarker measurement with specificity for SAA, HNL, GDF 15, IgA, Fibronectin, A1AT, YKL-40, or PCT;
Selection of at least one biomarker measurement with specificity for Leptin, IgE, Eotaxin, C1q, sST2, MMP-9, Neutrophil Elastase, or Resistin;
Optionally selection of at least one of the four biomarkers may have a non-monotonic contribution to the disease score, where the at least one biomarker is selected from sRAGE, Leptin, adiponectin, Pentraxin 3, YKL40, GDF 15, PARC, Fibronectin, IgE, Eotaxin, Cystatin C, NT-proANP, TIMP1, and D-Dimer;
Optionally selection of at least one biomarker indicative of a contribution from at least one protein complex is included, where specificity for at least one complex component is selected from A1AT, IgA, C1q, CRP, PTX3, sRAGE, (HMGB1, calprotectin), PF4, RANTES, Cystatin C, MMP-9, TIMP-1, and YKL-40;
Optionally selection of at least one pulmonary function test variable is included in the disease score, specifically FEV1/FVC, FEV1 in liters, FVC in liters, FEV1 in percent predicted value, FEV1 reversibility, and residual volume/total lung capacity ratio;
Optionally selection of at least one quantitative CT measure is included in the disease score, specifically emphysema by low area attenuation percentage <−950 Hounsfield Units, or a measure of small airways disease, for example percentage <−856 HU in the small airways, gas trapping or hyperinflation by measure of residual illuminated volumes at maximum expiration.
Optionally selection of at least one score representative of symptoms is included, specifically a score of dyspnea, dyspnea on exertion, dyspnea on performing daily activities, cough, phlegm production, chest tightness, sleep quality, energy level and confidence levels
Optionally selection of at least one variable representative of the patients' exacerbations history, occurrence in the past month, 3 months, 6 months 12 months, 18 months, number occurred within these time frames, and urgency in the form of setting of care received, out-patient call in, phone video, or clinic visit, emergency department use, hospital admission, hospital admission with intubation.
Optionally selection of at least one variable representative of patient/subject demographic: age, sex, or race.
Optionally selection of at least one variable representative of patient/subject risk factors: smoking or exposure history, active or inactive, body mass, body mass index.
Optionally selection variables representative of current medications use: steroids, LABA, LAMA, PDE inhibitors, anti-inflammatory, antibiotics such as chronic use of low dose macrolides, biologics targeted to interfere with immunological pathways, complement pathway inhibitors, and supplements and augmentations for deficiencies, and combinations.
Optionally selection of a variable representative of a comorbid condition such as metabolic disorder, a vascular, circulatory, cardiac, additional lung, liver, or gastrointestinal or CNS disorder.
Optionally selection of a variable representative of time of year or season.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-40. (canceled)

41. A method of detecting protein, comprising:
(a) obtaining a biological sample from a subject, wherein said biological sample comprises proteins and wherein said subject has or is suspected of having chronic obstructive pulmonary disease (COPD);
(b) detecting a level of at least four proteins, wherein
(i) at least one protein of said at least four proteins is selected from the group consisting of: Soluble Receptor for Advanced Glycation End products (sRAGE), Platelet Factor-4 (PF4), P-selectin, Regulated on Activation Normal T Cell Expressed and Secreted (RANTES), Tissue Inhibitor of Metalloproteinases 1 (TIMP1), Pulmonary and Activation-Regulated Chemokine (PARC), Club cell 16 protein (CC16), Pro-peptide of Atrial natriuretic Peptide (NT-proANP), and Fibrinogen;
(ii) at least one protein of said at least four proteins is selected from the group consisting of: C-reactive protein (CRP), Pentraxin 3 (PTX3), Adiponectin, D-Dimer, Interleukin 6 (IL6), Monocyte Chemoattractant Protein 1 (MCP-1), Cathepsin S, and Cystatin C;
(iii) at least one protein of said at least four proteins is selected from the group consisting of: Serum amyloid A (SAA), Human Neutrophil Lipocalin (HNL), Growth Differentiation Factor 15 (GDF 15), Immunoglobulin A (IgA), Fibronectin, Alpha-1 antitrypsin (A1AT), Chitinase 3-like 1 (YKL-40), and Procalcitonin (PCT); and
(iv) at least one protein of said at least four proteins is selected from the group consisting of: Leptin, Immunoglobulin E (IgE), Eotaxin, Complement component 1q (C1q), soluble Interleukin 1 Receptor-like 1 (sST2), Matrix Metallopeptidase 9 (MMP-9), Neutrophil Elastase, and Resistin.

42. The method of claim 41, wherein said biological sample is selected from the group consisting of: blood, plasma, serum, dried blood spot, bronchial lavage, nasal swab, saliva, breath condensate, sputum, and a combination thereof.

43. The method of claim 41, wherein said method further comprises identifying said subject as part of a population based on a level of said at least four proteins, wherein said population is selected from a group consisting of: a population with controlled chronic obstructive pulmonary disease, a population with uncontrolled chronic obstructive pulmonary disease, a population prone to a future acute exacerbation event, a population not prone to a future acute exacerbation event, a population which will benefit from an increased therapy, a population which will benefit from a decreased therapy, and a combination thereof.

44. The method of claim 41, wherein at least one of said proteins is a protein complex component.

45. The method of claim 44, wherein said protein complex component is selected from the group consisting of: A1AT, IgA, C1q, CRP, PTX3, sRAGE, HMGB1, calprotectin, PF4, RANTES, Cystatin C, MMP-9, TIMP-1, and YKL-40.

46. The method of claim 41, wherein at least one of said proteins is selected from the group consisting of: sRAGE, Leptin, adiponectin, PTX3, YKL40, GDF 15, PARC, Fibronectin, IgE, Eotaxin, Cystatin C, NT-proANP, TIMP1, and D-Dimer.

47. The method of claim 41, further comprising, measuring or determining an additional parameter of the subject.

48. The method of claim 47, wherein the additional parameter is selected from the group consisting of:
(a) a pulmonary function test variable, wherein said pulmonary function test variable is selected from the group consisting of: a ratio of forced expiratory volume in 1 second (FEV1) to a forced vital capacity (FVC), FEV1 in liters, FVC in liters, FEV1 in percent predicted value, FEV1 reversibility, residual volume/total lung capacity ratio, peak flow, and any combination thereof;
(b) a quantitative computed tomography measure, wherein said quantitative computed tomography measure is selected from the group consisting of: Low Attenuation Area at max inspiration, Low Attenuation Area at max expiration, airway wall area, airway wall thickness, a parametric measure of emphysema or small airway disease, and any combination thereof;
(c) a score representative of a symptom, wherein said symptom is selected from the group consisting of: dyspnea, dyspnea on exertion, dyspnea on performing daily activities, cough, phlegm production, chest tightness, sleep quality, energy level, confidence level, and any combination thereof;
(d) a variable representative of an exacerbation history of said subject, wherein said exacerbation history is selected from the group consisting of: an exacerbation occurrence in a given time frame, a form of setting of care received, a care received, and any combination thereof;
(e) a variable representative of a demographic of said subject, wherein said demographic is selected from the group consisting of: age, sex, race, and any combination thereof;
(f) a variable representative of a risk factor of said subject, wherein said risk factor is selected from the group consisting of: smoking, smoking exposure, activity level, body mass, body mass index, and any combination thereof;
(g) a variable representative of use of a medication of said subject, wherein said medication is selected from the group consisting of: a steroid, a long-acting beta-agonist, a long-acting muscarinic antagonist, a phosphodiesterase inhibitor, an anti-inflammatory, an antibiotic, a supplement, and any combination thereof; and
(h) a variable representative of a comorbid condition of said subject, wherein said comorbid condition is selected from the group consisting of: a metabolic disorder, a vascular disorder, a circulatory disorder, a cardiac disorder, a non-chronic obstructive pulmonary disease lung disorder, a liver disorder, a gastrointestinal disorder, a central nervous system disorder, and any combination thereof.

49. The method of claim 41, further comprising, administering or recommending an intervention to said subject based on said level of at least four proteins.

50. The method of claim 49, wherein said intervention is a treatment selected from Table 1.

51. The method of claim 41, wherein said detecting of (b) further comprises performing an immunoassay to detect a level of at least one protein of said at least four proteins.

52. The method of claim 51, wherein said immunoassay is selected from the group consisting of: enzyme-linked immunosorbent assay (ELISA), homogeneous immunoassay, Western blot, fluorescence immunoassay, chemiluminescence immunoassay, electro-chemiluminescence immunoassay, fluorescence resonance energy transfer (FRET) immunoassay, time resolved fluorescence and/or FRET immunoassay, lateral flow immunoassay, microspot (fluorescence) immunoassay, surface plasmon resonance immunoassays, ligand assay, clotting assay, immune-capture coupled with mass spectrometry, and non-optical immunoassay.

53. The method of claim 52, wherein said non-optical immunoassay is an acoustic membrane microparticle (AMMP) assay.

54. The method of claim 51, wherein said immunoassay is performed using one or more antibodies specific for said at least one protein.

55. The method of claim 54, wherein said one or more antibodies comprise a detectable label.

56. The method of claim 55, wherein said detectable label comprises a fluorescent label, an enzymatic label, or a small molecule label.

57. The method of claim 54, further comprising, detecting said one or more antibodies with a labeled detection antibody.

58. A kit for detecting proteins, comprising:
(a) a first antibody for detecting a level of one or more first proteins selected from the group consisting of: sRAGE, PF4, P-selectin, RANTES, TIMP1, PARC, CC16, NT-proANP, and Fibrinogen;
(b) a second antibody for detecting a level of one or more second proteins selected from the group consisting of: CRP, Pentraxin 3, Adiponectin, D-Dimer, IL6, MCP-1, Cathepsin S, and Cystatin C;
(c) a third antibody for detecting a level of one or more third proteins selected from the group consisting of: SAA, HNL, GDF 15, IgA, Fibronectin, A1AT, YKL-40, and PCT;
(d) a fourth antibody for detecting a level of one or more fourth proteins selected from the group consisting of: Leptin, IgE, Eotaxin, C1q, sST2, MMP-9, Neutrophil Elastase, and Resistin; and
(e) instructions for using said first antibody, second antibody, third antibody, and fourth antibody in an assay for detecting proteins.
